(19) United States
(12) Patent Application Publication
(10) Pub. No.: US 2026/0069604 A1
(43) Pub. Date: Mar. 12, 2026

(54) NOVEL EQUILIBRATIVE NUCLEOSIDE TRANSPORTER INHIBITORS AND METHODS OF MAKING AND USING SAME

(71) Applicant: Duke University, Durham, NC (US)

(72) Inventors: Seok-Yong Lee, Durham, NC (US); Jiyong Hong, Durham, NC (US); Ru-Rong Ji, Durham, NC (US); Nicholas Wright, Durham, NC (US)

(21) Appl. No.: 18/869,423

(22) PCT Filed: Jun. 1, 2023
(86) PCT No.: PCT/US2023/024118

§ 371 Date: Nov. 26, 2024

Related U.S. Application Data
(60) Provisional application No. 63/347,735, filed on Jun. 1, 2022.

(51) Int. Cl. A61K 31/551; A61K 31/341; A61P 25/02; C07D 245/02; C07D 307/20; C07D 405/12; C07D 498/08

(52) U.S. Cl. CPC A61K 31/551; A61K 31/341; A61P 25/02; C07D 245/02; C07D 307/20; C07D 405/12; C07D 498/08

(57) ABSTRACT

Described herein are equilibrative nucleoside transporter inhibitors and methods of making and using same. In some embodiments, the inhibitors are used for the prevention and/or treatment of pain.

### CROSS-REFERENCE TO RELATED APPLICATIONS

This application claims priority to U.S. Provisional Patent Application No. 63/347,735 filed on Jun. 1, 2022, which is incorporated by reference herein in its entirety.

### FEDERALLY SPONSORED RESEARCH

This invention was made with government support under grant numbers R01GM137421 awarded by the National Institutes of Health (NIH) and W81XWH2110756 by the Department of Defense (DoD). The Federal Government has certain rights to this invention.

### BACKGROUND

There is an urgent need for new non-opioid analgesic agents that are safe and effective for treating acute and chronic pain including neuropathic pain. The nucleoside adenosine is an endogenous neuromodulator in central and peripheral nervous systems. It is well known that adenosine inhibits pain by activating the adenosine 1 receptor (A1R), which produces postsynaptic inhibition of pain signal transmission. Many previous efforts to develop agonists for the A1 receptor have not progressed clinically because highly potent A1R agonist leads to undesirable side effects and partial A1R agonists show insufficient efficacy. Attention has been recently diverted toward utilizing endogenous adenosine signaling for pain suppression. Prolonged adenosine signaling achieved through its cellular reuptake inhibition is another route to activate A1R. The equilibrative nucleoside transporter 1 (ENT1) is the major adenosine transporter in humans. Inhibition of ENT1 will increase extracellular adenosine concentration, which in turn activates adenosine receptor signaling. This concept bears similarity to the mode of action of antidepressant on serotonin transporters. In fact, the ENT1 inhibitors dipyridamole and dilazep have been clinically used as vasodilators. Despite the promise, there have not been significant efforts to develop ENT1 inhibitors as novel analgesics.

What is needed are equilibrative nucleoside transporter inhibitors and methods of making and using the same for the prevention or treatment of pain.

### SUMMARY

One embodiment described herein is a compound of formula (I), or a pharmaceutically acceptable salt thereof:

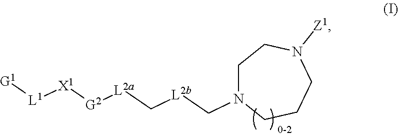

wherein:
G¹ is a 6- to 12-membered aryl or a 5- to 12-membered heteroaryl, wherein G¹ is optionally substituted with 1-4 R^{1x}, wherein, at each occurrence, R^{1x} is independently —NO₂, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, or —C₁₋₃alkylene-Q^{1a};
R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{1a} at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L¹ is C₁₋₄alkylene or absent;
X¹ is O, S, or NH;
G² is phenylene, wherein G² is optionally substituted with 1-4 R^{2x}, wherein, at each occurrence, R^{2x} is independently halogen, C₁₋₆alkyl, C₁₋₆haloalkyl, C₂₋₆alkenyl, —OR^{2a}, —NO₂, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, or —C₁₋₃alkylene-Q^{2a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a};
R^{2d}, at each occurrence, is independently C₁₋₆alkyl, C₁₋₆haloalkyl, G^{2a}, or —C₁₋₃alkylene-G^{2a};
G^{2a}, at each occurrence, is independently a C₃₋₆cycloalkyl, a phenyl, a 4- to 6-membered heterocyclyl, or a 5- to 6-membered heteroaryl; wherein G^{2a} is optionally substituted with 1-5 substituents independently selected from the group consisting of halogen, oxo, C₁₋₄alkyl, —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{2a}, at each occurrence, is independently —OC₁₋₄alkyl, —OC₁₋₄haloalkyl, —OH, —NH₂, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L^{2a} is

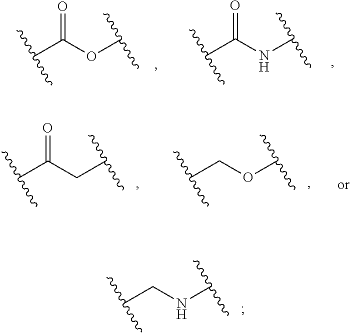

L^{2b} is C₁₋₃alkylene, C₂₋₃alkenylene, C₂₋₃alkynylene, or absent;
Z¹ is

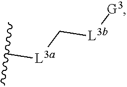

C₁₋₆alkyl, or C₃₋₈cycloalkyl;
L^{3a} is C₁₋₃alkylene, C₂₋₃alkenylene, C₂₋₃alkynylene, or absent; and
L^{3b} is

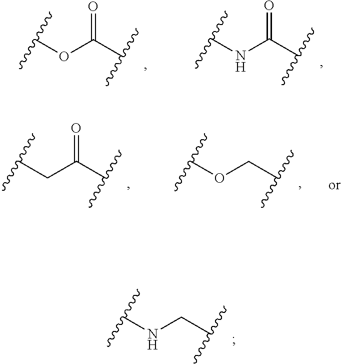

G³ is a 6- to 12-membered aryl or a 5- to 12-membered heteroaryl, wherein G³ is optionally substituted with 1-4 R^{3x}, wherein, at each occurrence, R^{3x} is independently halogen, C₁₋₆alkyl, C₁₋₆haloalkyl, C₂₋₆alkenyl, —OR^{3a}, —NO₂, —NR^{3a}R^{3b}, —SR^{3a}, —NR^{3a}C(O)R^{3c}, cyano, —C(O)OR^{3a}, —C(O)NR^{3a}R^{3b}, —C(O)R^{3c}, —SO₂R^{3d}, —SO₂NR^{3a}R^{3b}, G^{3a}, —C₁₋₃alkylene-G^{3a}, or —C₁₋₃alkylene-Q^{3a};
R^{3a}, R^{3b}, and R^{3c}, at each occurrence, are each independently hydrogen, C₁₋₆alkyl, C₁₋₆haloalkyl, G^{3a}, or —C₁₋₃alkylene-G^{3a};
R^{3d}, at each occurrence, is independently C₁₋₆alkyl, C₁₋₆haloalkyl, G^{3a}, or —C₁₋₃alkylene-G^{3a};
G^{3a}, at each occurrence, is independently a C₃₋₈cycloalkyl, a 4- to 12-membered heterocyclyl, a 6- to 12-membered aryl, or a 5- to 12-membered heteroaryl, wherein G^{3a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂; and
Q^{3a} at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂.

In one aspect, the compound is a compound of formula (I-a):

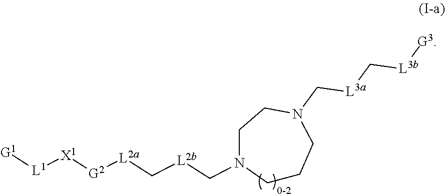

In another aspect, G¹ is phenyl.

In another aspect, G¹ is substituted with 1-3 R^{1x}, wherein, at each occurrence, R^{1x} is independently —NO₂ or C₁₋₄alkyl.

In another aspect, G¹ is

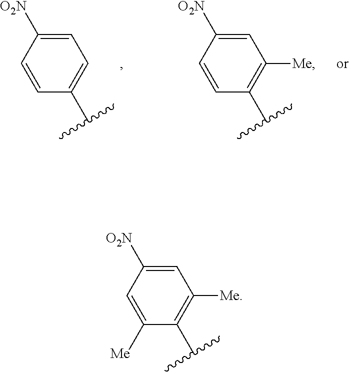 [SMILES: *c1ccc([N+](=O)[O-])cc1 ; *c1ccc([N+](=O)[O-])cc1C ; *c1c(C)cc([N+](=O)[O-])cc1C]

In another aspect, L¹ is C₁₋₃alkylene.

In another aspect, X¹ is O or S.

In another aspect, G² is substituted with 1-3 R^{2x}, wherein, at each occurrence, R^{2x} is —OR^{2a}.

In another aspect, R^{2a} is C₁₋₄alkyl, cyclopropyl, or —C₁₋₃alkylene-G^{2a}.

In another aspect, at least one R^{2a} is methyl, ethyl, or isopropyl.

In another aspect, G^{2a} is

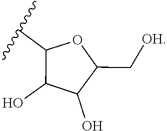 [SMILES: *C1OC(CO)C(O)C1O]

In another aspect, L^{2a} is

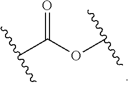 [SMILES: *OC(*)=O]

In another aspect, L^{2b} is C₁₋₃alkylene.

In another aspect,

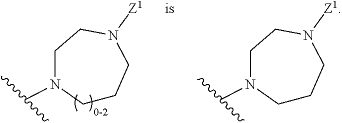

In another aspect, Z¹ is

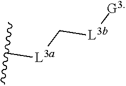

In another aspect, L^{3a} is C₁₋₃alkylene.

In another aspect, L^{3b} is

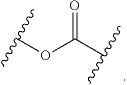 [SMILES: *OC(*)=O]

In another aspect, G³ is phenyl.

In another aspect, G³ is substituted with 1-3 R^{3x}, wherein R^{3x} is —OR^{3a}.

In another aspect, R^{3a} is C₁₋₄alkyl or cyclopropyl.

In another aspect, Z¹ is C₁₋₄alkyl or cyclopropyl.

In another aspect, the compound is:

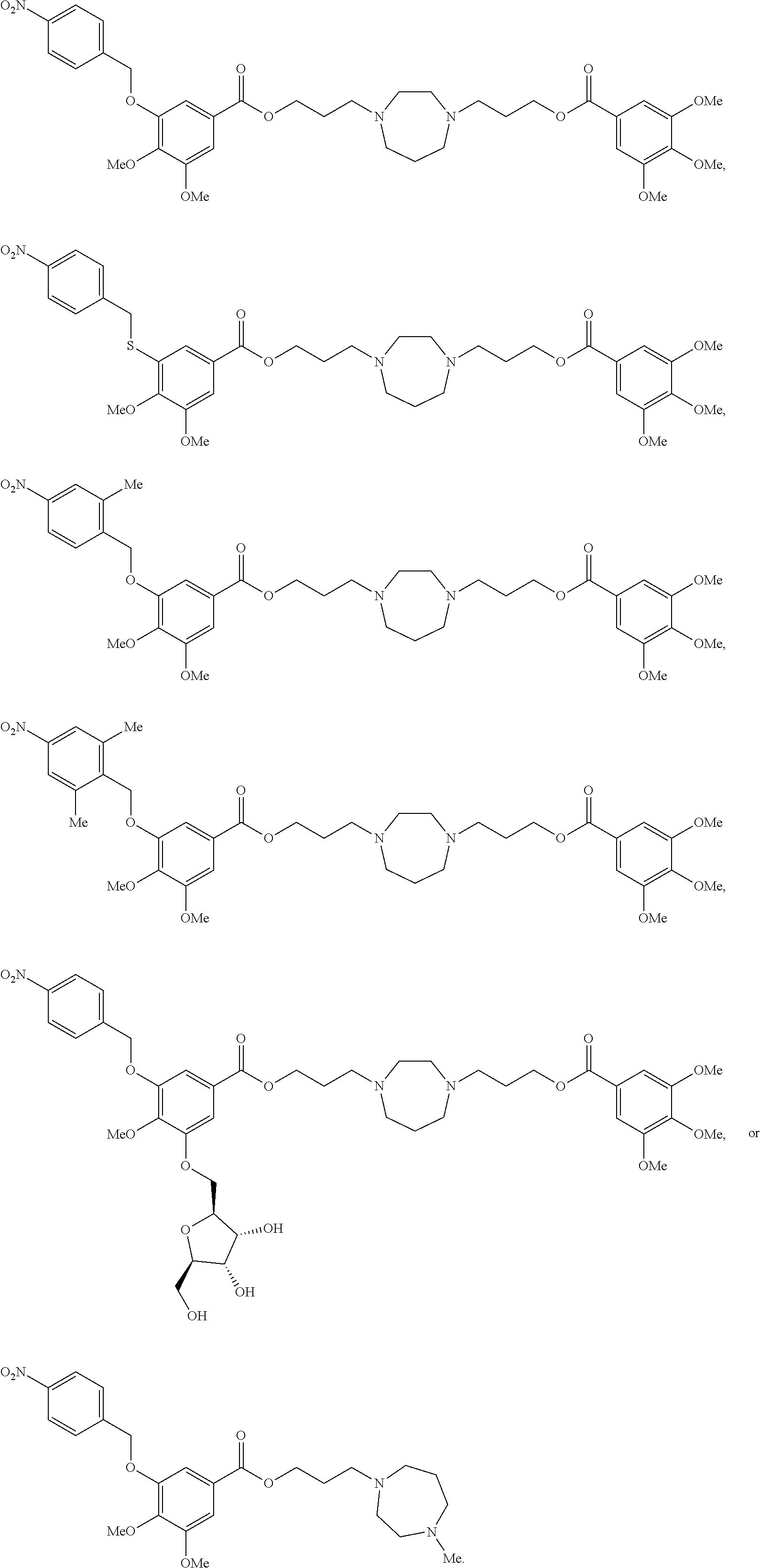

Another embodiment described herein is a pharmaceutical composition comprising a compound of formula (I), or a pharmaceutically acceptable salt thereof.

Another embodiment described herein is a method of treating neuropathic pain, the method comprising administering to a subject in need thereof a therapeutically effective amount of the compound of formula (I), or a pharmaceutically acceptable salt thereof, or a pharmaceutical composition comprising the compound of formula (I), or a pharmaceutically acceptable salt thereof.

Another embodiment described herein is a method of inhibiting an equilibrative nucleoside transporter (ENT), the method comprising administering to a subject in need thereof a therapeutically effective amount of the compound of formula (I), or a pharmaceutically acceptable salt thereof, or a pharmaceutical composition comprising the compound of formula (I), or a pharmaceutically acceptable salt thereof.

In one aspect, the equilibrative nucleoside transporter is an equilibrative nucleoside transporter 1 (ENT1).

Another embodiment described herein is a compound of formula (II), or a pharmaceutically acceptable salt thereof:

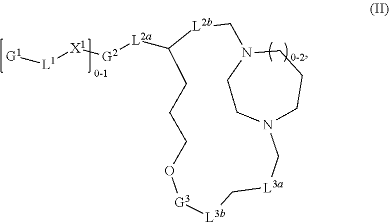

wherein:
G¹ is a 6- to 12-membered aryl or a 5- to 12-membered heteroaryl, wherein G¹ is optionally substituted with 1-4 R^{1x}, wherein, at each occurrence, R^{1x} is independently —NO₂, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, or —C₁₋₃alkylene-Q^{1a};
R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{1a}, at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄ alkyl, or —C(O)N(C₁₋₄alkyl)₂;
L¹ is C₁₋₄alkylene or absent;
X¹ is O, S, or NH;
when G¹-L¹-X¹ is present G² is phenylene;
when G¹-L¹-X¹ is absent G² is phenyl;
G² is optionally substituted with 1-4 R^{2x}, wherein, at each occurrence, R^{2x} is independently halogen, C₁₋₆alkyl, C₁₋₆haloalkyl, C₂₋₆alkenyl, —OR^{2a}, —NO₂, —NR^{2a}R^{2b}, —SR^{2a}, —NR^{2a}C(O)R^{2c}, cyano, —C(O)OR^{2a}, —C(O)R^{2c}, —C(O)NR^{2a}R^{2b}, —SO₂NR^{2a}R^{2b}, —SO₂R^{2d}, G^{2a}, —C₁₋₃alkylene-G^{2a}, or —C₁₋₃alkylene-Q^{2a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a};
R^{2d}, at each occurrence, is independently C₁₋₆alkyl, C₁₋₆haloalkyl, G^{2a}, or —C₁₋₃alkylene-G^{2a};
G^{2a}, at each occurrence, is independently a C₃₋₆cycloalkyl, a phenyl, a 4- to 6-membered heterocyclyl, or a 5- to 6-membered heteroaryl; wherein G^{2a} is optionally substituted with 1-5 substituents independently selected from the group consisting of halogen, oxo, C₁₋₄alkyl, —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{2a}, at each occurrence, is independently —OC₁₋₄alkyl, —OC₁₋₄haloalkyl, —OH, —NH₂, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L^{2a} is

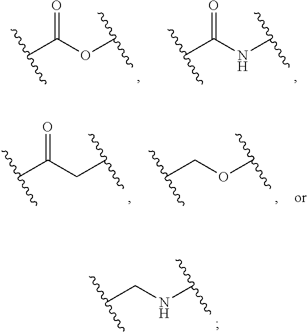

L^{2b} is C₁₋₃alkylene, C₂₋₃alkenylene, C₂₋₃alkynylene, or absent;
L^{3a} is C₁₋₃alkylene, C₂₋₃alkenylene, C₂₋₃alkynylene, or absent; and
L^{3b} is

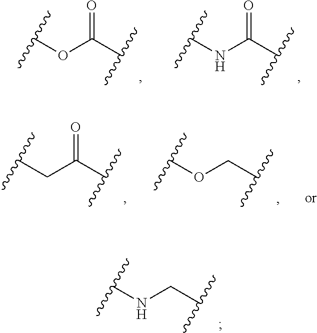

G³ is a 6- to 12-membered arylene or a 5- to 12-membered heteroarylene, wherein G³ is optionally substituted with 1-4 R^{3x}, wherein, at each occurrence, R^{3x} is independently halogen, C₁₋₆alkyl, C₁₋₆haloalkyl, C₂₋₆alkenyl, —OR^{3a}, —NO₂, —NR^{3a}R^{3b}, —SR^{3a}, —NR^{3a}C(O)R^{3c}, cyano, —C(O)OR^{3a}, —C(O)NR^{3a}R^{3b}, —C(O)R^{3c}, —SO₂R^{3d}, —SO₂NR^{3a}R^{3b}, G^{3a}, —C₁₋₃alkylene-G^{3a}, or —C₁₋₃alkylene-Q^{3a};
R^{3a}, R^{3b}, and R^{3c}, at each occurrence, are each independently hydrogen, C₁₋₆alkyl, C₁₋₆haloalkyl, G^{3a}, or —C₁₋₃alkylene-G^{3a};
R^{3d}, at each occurrence, is independently C₁₋₆alkyl, C₁₋₆haloalkyl, G^{3a}, or —C₁₋₃alkylene-G^{3a};
G^{3a}, at each occurrence, is independently a C₃₋₈cycloalkyl, a 4- to 12-membered heterocyclyl, a 6- to 12-membered aryl, or a 5- to 12-membered heteroaryl, wherein G^{3a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂; and
Q^{3a}, at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂.

In another aspect, the compound is a compound of formula (II-a) or (II-b):

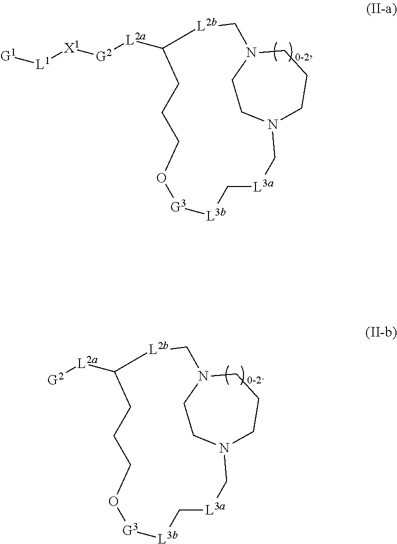

In another aspect, G¹ is phenyl.

In another aspect, G¹ is substituted with 1-3 R^{1x}, wherein, at each occurrence, R^{1x} is independently —NO₂ or C₁₋₄alkyl.

In another aspect, L¹ is C₁₋₄alkylene and X¹ is O or S.

In another aspect, G² is substituted with 1-3 R^{2x}, wherein, at each occurrence, R^{2x} is —OR^{2a}.

In another aspect, L^{2a} is

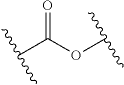 [SMILES: *OC(*)=O]

and L^{2b} is C₁₋₃alkylene.

In another aspect,

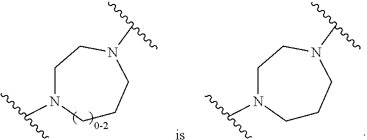

In another aspect, L^{3a} is C₁₋₃alkylene.

In another aspect, L^{3b} is

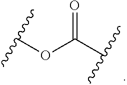 [SMILES: *OC(*)=O]

In another aspect, G³ is phenylene.

In another aspect, G³ is substituted with 1-3 R^{3x}, wherein R^{3x} is —OR^{3a}.

In another aspect, the compound is:

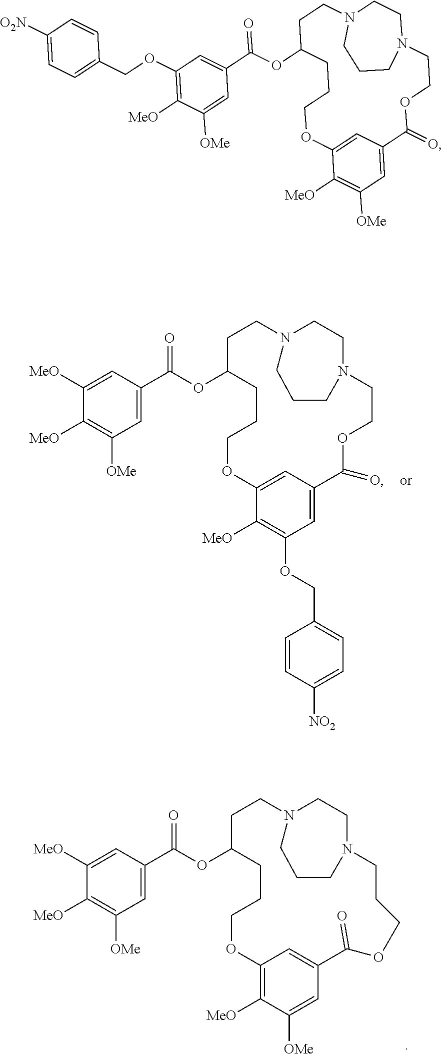 [SMILES: COc1cc2cc(c1OC)OCCCC(OC(=O)c1cc(OC)c(OC)c(OCc3ccc([N+](=O)[O-])cc3)c1)CCN1CCCN(CCOC2=O)CC1 ; COc1cc(C(=O)OC2CCCOc3cc(cc(OCc4ccc([N+](=O)[O-])cc4)c3OC)C(=O)OCCN3CCCN(CC2)CC3)cc(OC)c1OC ; COc1cc(C(=O)OC2CCCOc3cc(cc(OC)c3OC)C(=O)OCCCN3CCCN(CC2)CC3)cc(OC)c1OC]

Another embodiment described herein is a pharmaceutical composition comprising a compound of formula (II), or a pharmaceutically acceptable salt thereof.

Another embodiment described herein is a method of treating neuropathic pain, the method comprising administering to a subject in need thereof a therapeutically effective amount of the compound of formula (II), or a pharmaceutically acceptable salt thereof, or a pharmaceutical composition comprising the compound of formula (II), or a pharmaceutically acceptable salt thereof.

Another embodiment described herein is a method of inhibiting an equilibrative nucleoside transporter (ENT), the method comprising administering to a subject in need thereof a therapeutically effective amount of the compound of formula (II), or a pharmaceutically acceptable salt thereof, or a pharmaceutical composition comprising the compound of formula (II), or a pharmaceutically acceptable salt thereof.

Another embodiment described herein is a compound of formula (III), or a pharmaceutically acceptable salt thereof:

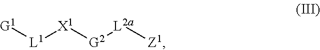

wherein:
G¹ is a 6- to 12-membered aryl or a 5- to 12-membered heteroaryl, wherein G¹ is optionally substituted with 1-4 R^{1x}, wherein, at each occurrence, R^{1x} is independently —NO₂, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, or —C₁₋₃alkylene-Q^{1a};
R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{1a}, at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L¹ is C₁₋₄alkylene or absent;
X¹ is O, S, or NH;
G² is phenylene, wherein G² is optionally substituted with 1-4 R^{2x}, wherein, at each occurrence, R^{2x} is independently halogen, C₁₋₆alkyl, C₁₋₆haloalkyl, C₂₋₆alkenyl, —OR^{2a}, —NO₂, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, or —C₁₋₃alkylene-Q^{2a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a};
R^{2d}, at each occurrence, is independently C₁₋₆alkyl, C₁₋₆haloalkyl, G^{2a}, or —C₁₋₃alkylene-G^{2a};
G^{2a}, at each occurrence, is independently a C₃₋₆cycloalkyl, a phenyl, a 4- to 6-membered heterocyclyl, or a 5- to 6-membered heteroaryl; wherein G^{2a} is optionally substituted with 1-5 substituents independently selected from the group consisting of halogen, oxo, C₁₋₄alkyl, —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{2a}, at each occurrence, is independently —OC₁₋₄alkyl, —OC₁₋₄haloalkyl, —OH, —NH₂, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L^{2a} is

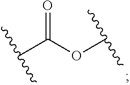 [SMILES: *OC(*)=O]

and
Z¹ is C₁₋₆alkyl, or C₃₋₆cycloalkyl.

In another aspect, G¹ is

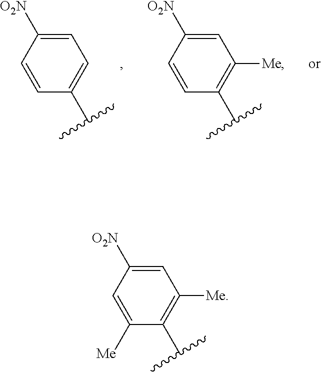 [SMILES: *c1ccc([N+](=O)[O-])cc1 ; *c1ccc([N+](=O)[O-])cc1C ; *c1c(C)cc([N+](=O)[O-])cc1C]

In another aspect, G² is

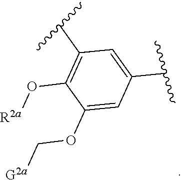

In another aspect, the compound is:

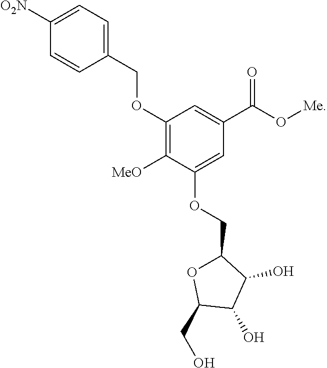 [SMILES: COC(=O)c1cc(OCc2ccc([N+](=O)[O-])cc2)c(OC)c(OC[C@@H]2O[C@H](CO)[C@@H](O)[C@H]2O)c1]

Another embodiment described herein is a pharmaceutical composition comprising a compound of formula (III), or a pharmaceutically acceptable salt thereof.

Another embodiment described herein is a method of treating neuropathic pain, the method comprising administering to a subject in need thereof a therapeutically effective amount of the compound of formula (III), or a pharmaceutically acceptable salt thereof, or a pharmaceutical composition comprising the compound of formula (III), or a pharmaceutically acceptable salt thereof.

Another embodiment described herein is a method of inhibiting an equilibrative nucleoside transporter (ENT), the method comprising administering to a subject in need thereof a therapeutically effective amount of the compound of formula (III), or a pharmaceutically acceptable salt thereof, or a pharmaceutical composition comprising the compound of formula (III), or a pharmaceutically acceptable salt thereof.

In another aspect, the equilibrative nucleoside transporter is equilibrative nucleoside transporter 1 (ENT1).

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows endogenous and exogenous nucleosides. Select endogenous nucleoside shown at top, with nucleoside/nucleotide antiviral and anticancer drugs shown at bottom.

FIG. 2 shows adenosine reuptake inhibitors. Chemical structures of select adenosine reuptake inhibitors (AdoRIs).

FIG. 3A-C show functional properties of hENT1_{cryst}. FIG. 3A shows uptake of 0.2 mM ³H-uridine in 90 seconds into hENT1 reconstituted proteoliposomes loaded with 1.0 mM cold uridine (n=11 replicates, across biological triplicate, error bars ±S.E.M. ** denotes p<0.01 and **** denotes p<0.0001 from unpaired two-tailed t-test). FIG. 3B shows uptake of 1.0 mM ³H-adenosine in 10 minutes into X. laevis oocytes expressing wild type hENT1 and hENT1_{cryst} (n=7-11 technical replicates, across biological duplicate, error bars ±S.E.M. **** denotes p<0.0001 from unpaired two-tailed t-test). FIG. 3C shows saturation binding of ³H-nitrobenzylthioinosine (NBMPR) to detergent purified wild type hENT1 and hENT1_{cryst} (10 nM GFP-FLAG-His₁₀-fusion transporter) with scintillation proximity (biological triplicate of technical duplicates, error bars ±S.E.M. from n=6 measurements).

FIG. 4A-F show experimental phasing and structure determination of hENT1_{cryst}. FIG. 4A shows a representative gel filtration profile of DDM detergent purified hENT1_{cryst} in the presence of dilazep. FIG. 4B shows hENT1_{cryst}+dilazep form plate-like crystals in lipidic cubic phase. FIG. 4C shows the final model in the initial density modified SIRAS map (1a) to 3.5 Å. FIG. 4D shows the final model in 2Fₒ-F_{c} composite omit calculated map (1σ), to 2.3 Å. FIG. 4E shows an Fₒ-F_{c} simulated annealing omit map for dilazep (36) to 2.3 Å, calculated using a starting temperature of 2,500K. FIG. 4F shows a simple Fₒ-F_{c} omit density corresponding to NBMPR (26 contour, left) and Fₒ-F_{c} simulated annealing omit map for NBMPR to 2.9 Å (2σ contour, right), calculated using a starting temperature of 2,500K.

FIG. 5 shows the structural architecture of the ENT fold. Diagram depiction of the membrane topology of human ENT1 (top) and cartoon representation of the dilazep-bound hENT1_{cryst} structure (bottom).

FIG. 6 shows a comparison of ENT and MFS folds. Structural superposition of the hENT1_{cryst} structure (shown in red) with a structure of a representative MFS member, hGlut3 (shown in blue).

FIG. 7 shows pseudo-symmetry in the ENT fold. Structural superposition of the N- and C-domains of hENT1_{cryst} highlight symmetric and asymmetric features within the ENT protein fold.

FIG. 8A-B show the outward-facing state of human ENT1. FIG. 8A shows both structures feature hENT1_{cryst} in an outward-facing conformation. FIG. 8B shows hydrophobic residues that form the extracellular and intracellular transporter gates are shown in grey, with residues participating in polar/charged interactions shown as sticks.

FIG. 9 shows the interaction network at the intracellular gate. The structural position in the hENT1_{cryst} structure that is analogous to the “A-motif” in MFS is shown at left, with a representative MFS transporter (hGlut3) shown at right.

FIG. 10 shows dilazep binding to hENT1. Detailed transporter-inhibitor interactions within the dilazep binding site, with important residues shown as sticks. Chemical structure of dilazep shown above for reference.

FIG. 11 shows NBMPR binding to hENT1. Detailed transporter-inhibitor interactions within the NBMPR binding site, with important residues shown as sticks. Chemical structure of NBMPR shown above for reference.

FIG. 12 shows structural modeling of G154S. Slice-through view of central cavity, from the NBMPR bound hENT1_{cryst} structure (left). The G154S mutation was introduced into this structure using PyMOL (right), highlighting the collapse of opportunistic site 2 upon substitution with serine.

FIG. 13A-D show shared and distinct sites of dilazep and NBMPR. FIG. 13A shows the NBMPR and dilazep binding sites with residues of interest shown as sticks. FIG. 13B shows equilibrium binding of ³H-NBMPR to detergent purified hENT1_{cryst} and mutants. FIG. 13C shows cold competition displacement of ³H-NBMPR by dilazep from hENT1_{cryst} and mutants (error bars S.E.M., for biological triplicate of technical duplicate) FIG. 13D shows fold-changes in binding from panels B and C, shown per experimental date (error bars S.E.M., for biological triplicate).

FIG. 14 shows structural mechanisms of action for dilazep and NBMPR. Proposed inhibitory mechanisms of action employed by dilazep and NBMPR. While dilazep sterically blocks extracellular gate closure, NBMPR disrupts the transition from outward-occluded to inward facing conformational states.

FIG. 15A-B show the conceptual basis for rational AdoRI design. FIG. 15A shows structural superposition of the two AdoRIs, with dilazep colored in green and NBMPR colored in dark red. FIG. 15B shows chemical structures of dilazep and the novel AdoRI JH-ENT-01.

FIG. 16A-B show the X-ray structure of JH-ENT-01 bound hENT1_{cryst}. FIG. 16A shows JH-ENT-01 ligand unmodelled Fₒ-F_{c} density shown at a contour of 3s. FIG. 16B shows the structural superposition of the NBMPR bound and JH-ENT-01 bound hENT1_{cryst} structures.

FIG. 17 shows JH-ENT-01 binding to detergent purified hENT1_{cryst}. The binding of cold competitor (dilazep control or JH-ENT-01) to hENT1_{cryst} (85 nM) was assessed by the displacement of ³H-NBMPR (10 nM) from the transporter (n=6 technical replicates from two representative experiments shown, error bars represent S.E.M.) FIG. 18A-C show initial functional characterization of novel hENT1 inhibitors. FIG. 18A shows chemical structures of the initial set of novel NBMPR-DZ hybrid analogs. FIG. 18B shows a summary of measured K_{d} values from cold competition SPA (n=9 technical replicates from three independent experiments for dilazep and JH-ENT-01, n=3 technical replicates from a single experiment for JH-ENT-02 and JH-ENT-03, error bars represent S.E.M.) FIG. 18C shows time-dependent uptake of ³H-ribavirin for oocytes expressing hENT1, hENT2 or hENT3 (N-terminal organellar retention sequence of hENT3 removed to allow for oocyte surface localization; n=1 technical replicates).

FIG. 19 shows hENT1 and hENT2 inhibition by dilazep and JH-ENT-01. Initial experiment (n=1 replicates) showing JH-ENT-01 inhibits both hENT1 and hENT2 mediated uptake of ³H-adenosine.

FIG. 20A-B show ENT1 expression in mouse DRG and spinal cord at two magnification levels (FIG. 20A and FIG. 20B).

FIG. 21A-F show the effects of intrathecal (i.t.) injection of ENT1 inhibitors on formalin-induced inflammatory pain in Phase 1 (0-10 min) and Phase 2 (10-45 min). Two-Way ANOVA followed by Bonferroni posthoc comparison. FIG. 21A shows males. FIG. 21B shows females. FIG. 21C shows both sexes. FIG. 21D-F shows duration of licking and flinching as a function of phase. *p<0.05, **p<0.01, ***p<0.001, ****p<0.0001, n=4 per sex, n=8 of both sexes. The inhibitors were given 30 min prior to intraplantar injection of formalin (5%, 20 μL).

FIG. 22A-C shows the effects of intrathecal (FIG. 22A, i.t., 10 nmol) injection or intravenous injections (FIG. 22C, i.v., 30 mg/kg) injections of ENT1 inhibitors on STZ-induced neuropathic pain (mechanical allodynia) in mice. Inhibitors and vehicle were administrated one week after STZ injection. FIG. 22A shows two-way ANOVA, Repeated Measures, F_{[8,27]}=3.619, p=0.0056. FIG. 22B shows the chemical structures of JH-ENT-04 and JH-ENT-07. FIG. 22C shows two-Way ANOVA followed by posthoc comparison. *p<0.05, **p<0.01, n=4 males in A. n=3-7 males in B. Arrows in B indicate iv. injections on STZ days 8, 10, and 12.

FIG. 23A-B show the effects of ENT1 inhibitor on sEPSCs in spinal cord slices of STZ-treated in mice. Spinal cord slices were prepared one week after STZ injection. FIG. 23A shows a schematic of patch clamp recordings in spinal cord slice and sEPSC traces before, during, and after the inhibitor perfusion (3 μM, 3 min). FIG. 23B shows quantification of sEPSC frequency (left) and amplitude (right) before and after JH-ENT-01 treatment. n=13 neurons from 3 male mice, paired Student's t-test.

FIG. 24 shows the effect of A1R antagonist CPCPX (i.t., 10 nmol) on preventing ENT1 inhibitor (10 nmol)-induced pain relief in STZ-treated mice. CPCPX was given 30 min prior to the injection of ENT1 inhibitor. *p<0.05, Two-Way ANOVA followed by posthoc comparison. n=6 males per group.

FIG. 25 shows the effect of the ENT1 inhibitor JH-ENT-01 (i.t., 100 nmol) on reversing SNI-induced neuropathic pain measured by PWF. *p<0.05, Two-Way ANOVA followed by posthoc comparison. n=4 males per group.

FIG. 26 shows inhibitory activity IC₅₀'s of select AdoRIs against WT hENT1 mediated ³H-adenosine uptake activity (200 nM ³H-ado uptake in 30 minutes, inhibitors added at the start of uptake, n=3 biol. reps. with mean±S.E.M. shown).

FIG. 27A-D show plasma (FIG. 27A), brain (FIG. 27B), and spinal cord (FIG. 27C) pharmacokinetics (PK) of JH-ENT-01 and dilazep. FIG. 27D shows a table of pharmacokinetic parameters for both drugs and all tissues. Male CD-1 mice (n=3 per time-point) were dosed subcutaneously (inj. volume: 8 μL/g BW) with 30 mg/kg of JH-ENT-01 (free base) and 29 mg/kg (molar equivalent) of dilazep dihydrochloride. Both drugs were formulated in 5% DMSO, 20% PEG-400, 20% propylene glycol, and 55% saline. Blood and tissues were collected at 10 min, 0.5, 1.5, 4.5, 8, 12, and 24 h. Following collection, the animals were perfused with saline. Liquid chromatography-tandem-mass spectrometry (LC/MS/MS) was used to determine drug concentration in samples. Pharmacokinetic parameters were calculated by non-compartmental approach within Win-Nonlin software. JH-ENT-01 exhibits longer half-life and higher plasma AUC than dilazep. The distribution of both drugs into CNS tissue is high and is in equilibrium with plasma drug concentration (i.e., no drug accumulation in CNS).

### DETAILED DESCRIPTION

Disclosed herein are equilibrative nucleoside transporter (ENT) inhibitors and methods of making and using the same for the prevention or treatment of pain.

### Definitions

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

The present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, the term “administering” an agent, such as a therapeutic entity to an animal or cell, is intended to refer to dispensing, delivering, or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The compounds and/or pharmaceutical compositions provided herein are useful in treating and/or preventing diseases, disorders, and/or conditions associated with ENT1. As used herein, the term “associated with ENT1” refers to those diseases, disorders, and/or conditions in which the modulation of ENT1 may treat, ameliorate and/or prevent the disease, disorder, and/or condition. The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like. In some embodiments, the disease, disorder and/or condition comprises pain. As used herein, the term “pain” refers to any type of pain (acute or chronic). Examples, include, but are not limited to, inflammatory pain, postoperative pain, neuropathic pain, cancer pain, and the like. In some embodiments, the pain comprises neuropathic pain after nerve injury, diabetes, chemotherapy, virus infection, stroke, and spinal cord injury.

“Contacting” as used herein, e.g., as in “contacting a sample” refers to contacting a sample directly or indirectly in vitro, ex vivo, or in vivo (e.g., within a subject as defined herein). Contacting a sample may include addition of a compound to a sample, or administration to a subject. Contacting encompasses administration to a solution, cell, tissue, mammal, subject, patient, or human. Further, contacting a cell includes adding an agent to a cell culture.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl” refers to a straight or branched hydrocarbon chain having one or more double bonds. Alkenyl groups may include a specified number of carbon atoms. For example, C₂-C₁₂ alkenyl indicates that the alkenyl group may have 2, 3, 4, 5, 6, 7, 8, 9, 10, 11 or 12 carbon atoms. An alkenyl group may be, e.g., a C₂-C₁₂ alkenyl group, a C₂-C₁₀ alkenyl group, a C₂-C₈ alkenyl group, a C₂-C₆ alkenyl group or a C₂-C₄ alkenyl group. Examples of alkenyl groups include but are not limited to allyl, propenyl, 2-butenyl, 3-hexenyl and 3-octenyl groups. One of the double bond carbons may optionally be the point of attachment of the alkenyl substituent. An alkenyl group may be optionally substituted with one or more substituents.

The term “alkenylenyl” refers to a divalent alkenyl group, examples of which include but are not limited to —CH═CH—, —CH═CH—CH₂—, —CH═CH—CH₂—CH₂— and —CH₂—CH═CH—CH₂—. An alkenylenyl group may be optionally substituted with one or more substituents.

The term “alkynyl” refers to a straight or branched hydrocarbon chain having one or more triple bonds. Alkynyl groups may include a specified number of carbon atoms. For example, C₂-C₁₂ alkynyl indicates that the alkynyl group may have 2, 3, 4, 5, 6, 7, 8, 9, 10, 11 or 12 carbon atoms. An alkynyl group may be, e.g., a C₂-C₁₂ alkynyl group, a C₂-C₁₀ alkynyl group, a C₂-C₈ alkynyl group, a C₂-C₆ alkynyl group or a C₂-C₄ alkynyl group. Examples of alkynyl groups include but are not limited to ethynyl, propargyl, and 3-hexynyl. One of the triple bond carbons may optionally be the point of attachment of the alkynyl substituent. An alkynyl group may be optionally substituted with one or more substituents.

The term “alkynylenyl” refers to a divalent alkynyl group, examples of which include but are not limited to —C≡C—, —C≡C—CH₂—, —C≡C—CH₂—CH₂— and —CH₂—C≡C—CH₂—. An alkynylenyl group may be optionally substituted with one or more substituents.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “alkylene,” as used herein, refers to a divalent group derived from a straight or branched chain hydrocarbon of 1 to 10 carbon atoms, for example, of 2 to 5 carbon atoms. Representative examples of alkylene include, but are not limited to, —CH₂—, —CD₂-, —CH₂CH₂—, —CH₂CH₂CH₂—, —CH₂CH₂CH₂CH₂—, and —CH₂CH₂CH₂CH₂CH₂—.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein. The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl).

Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl).

Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For purposes of illustration, examples of cycloalkylene and heterocyclylene include, respectively,

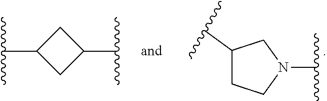 [SMILES: *C1CC(*)C1 ; *C1CCN(*)C1]

Cycloalkylene and heterocyclylene include a geminal divalent groups such as 1,1-C₃₋₆cycloalkylene (i.e.,

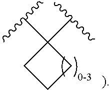

A further example is 1,1-cyclopropylene (i.e.,

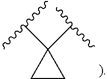 [SMILES: *C1(*)CC1]

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g., 1, 2, 3, or 4 heteroatoms independently selected from O, S, and N). The five membered aromatic monocyclic rings have two double bonds, and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10n electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “arylene” refers to a divalent group derived from an aromatic ring or ring system. For example, a “6-membered 1,4-arylene” is a divalent group derived from a monocyclic 6-membered aromatic ring having points of attachment or substitution on two carbon ring atoms in 1,4 relation to each other, i.e., para substitution, and a “6-membered 1,3-arylene” is a divalent group derived from a monocyclic 6-membered aromatic ring and having points of attachment or substitution on two carbon ring atoms in 1,3 relation to each other, i.e., meta substitution. For purposes of illustration, an example of a 6-membered 1,4-arylene is

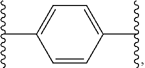 [SMILES: *c1ccc(*)cc1]

and an example of a 6-membered 1,3-arylene is

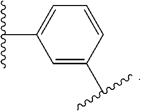 [SMILES: *c1cccc(*)c1]

The term “heteroarylene” refers to a divalent group derived from a heteroaromatic ring or ring system. For example, a “6-membered 1,4-heteroarylene containing 1-2 nitrogen atoms” is a divalent group derived from a monocyclic 6-membered heteroaromatic ring having 1-2 nitrogen atoms and having points of attachment or substitution on two carbon ring atoms in 1,4 relation to each other, i.e., para substitution. For purposes of illustration, examples of a 6-membered 1,4-heteroarylene containing 1-2 nitrogen atoms are

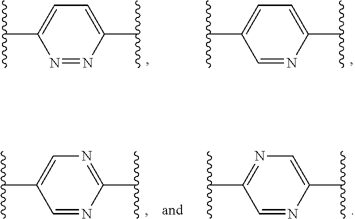 [SMILES: *c1ccc(*)nn1 ; *c1ccc(*)nc1 ; *c1cnc(*)nc1 ; *c1cnc(*)cn1]

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

### Nucleoside Transport Systems and Nucleoside Transporters

Nucleosides are involved in all aspects of human biology, serving as nucleotide precursors, required for nucleic acid synthesis and cellular metabolism. The nucleoside adenosine is a signaling molecule with its own dedicated cell surface receptors, which play essential roles in cellular response to hypoxic stress, neurotransmission, and neuromodulation. As nucleosides are small polar biomolecules, they rely on transport systems to cross biological membranes. Therefore, selective nucleoside transport regulates nucleoside-analog antiviral and antineoplastic drug absorption, delivery, metabolism, and excretion (ADME). Nucleoside transport in humans is mediated by the functions of integral membrane proteins known as nucleoside transporters (NTs). Due to the physiological and therapeutic importance of NTs, there have been numerous studies of the role of NTs in nucleoside-related physiology and drug efficacy, which have been extensively reviewed previously.

Cellular nucleoside pools are required for maintenance of energy stores, DNA/RNA synthesis, and cell signaling. Nucleoside levels are controlled via their phosphorylation in the generation of nucleotides and nucleic acids, or the hydrolysis of nucleotides via numerous enzymes. Adenosine is a purine nucleoside with multifaceted signaling roles, as it is a ligand for cell-surface adenosine G-protein coupled receptors (GPCRs). Concentrations of adenosine are typically in the nanomolar range, though certain conditions can trigger extracellular adenosine concentrations to exceed millimolar levels. Particularly, hypoxic/ischemic conditions can trigger the release of ATP into the extracellular matrix by pannexins, followed by hydrolysis of the nucleotides into adenosine by ectonucleotidases, leading to buildup of adenosine that ultimately initiates signaling through adenosine GPCRs. Subsequent signaling leads to vasodilation, angiogenesis, and metabolic regulation. Thus, adenosine is a protective agent in tissues undergoing hypoxic stress. Also involved in neurotransmission and neuromodulation, adenosine inhibits neuronal excitability, making it a key player in sleep/wakefulness, epilepsy, Alzheimer's disease, and substance abuse disorders.

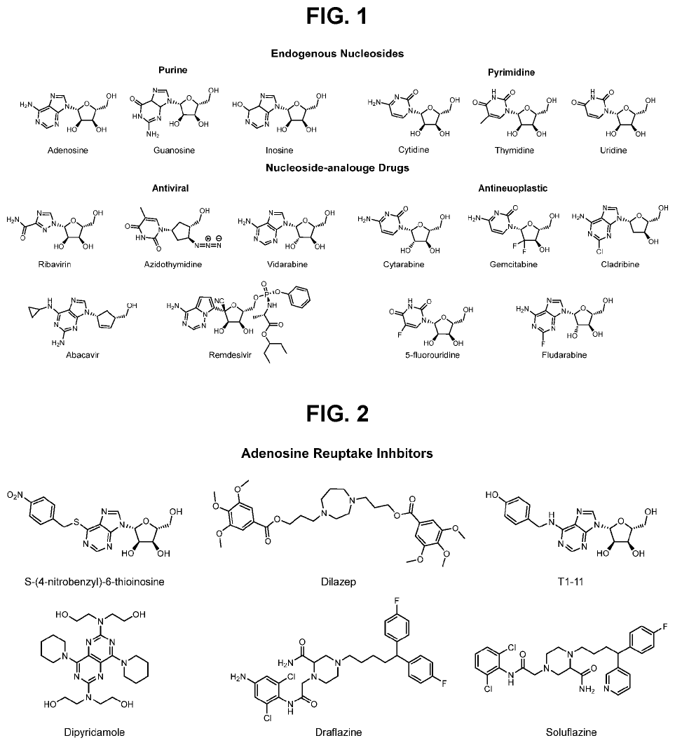

Disruption of cellular and viral replication through use of exogenous nucleoside-analogs (FIG. 1) has proven a viable therapeutic strategy in the treatment of cancers and viral infections, owing to the numerous metabolic roles that nucleosides play in the cell. Many nucleoside-analog anticancer drugs exhibit strong antimetabolite activities, attained by simple competition of endogenous nucleoside pools, which, depending on the specific drugs mechanism of action, leads to early chain termination, lesion incorporation into the genome, or aberrant DNA methylation, all of which result in general cytotoxicity. The selectivity of the nucleoside-analog anticancer drugs, albeit unexceptional in most cases, owes to the fact cellular replication levels are considerably higher in cancer cells than normal cells. Nucleoside-analog antiviral drugs function similarly, disrupting viral replication via inhibition of viral polymerases, chain termination or introduction of mutations into the viral genome. Particularly, studies have shown that numerous antiviral drugs can be rationally modified to inhibit specific viral polymerases, not endogenous human polymerases, therefore exhibiting suitable target specificities. Some nucleoside analog drugs that are commonly used in the clinic include azidothymidine (AZT)—a therapeutic used in the treatment of human immunodeficiency virus (HIV), cytarabine—a therapeutic used in treatment of leukemia, gemcitabine—a chemotherapeutic agent used in the treatment of pancreatic cancer, and ribavirin—a therapeutic used to treat hepatitis C. Of particular interest to recent events, several nucleoside-analogs have shown initial promise as antivirals in the treatment of novel coronavirus disease 2019 (COVID-19).

### Equilibrative Nucleoside Transporters

The four genes responsible for the sodium-independent nucleoside transport components were identified and classified as “equilibrative nucleoside transporters” (ENTs, gene family solute-carrier 29; SLC29). ENTs generally mediate selective, passive membrane transport of nucleosides. Responsible for es nucleoside transport, human ENT1 (hENT1) was the first human ENT member cloned and is most potently inhibited by NBMPR with the half maximal inhibitory concentration (IC₅₀) in the sub-nanomolar range. Cloned next, hENT2 was determined to be the major contributor to ei sodium-independent nucleoside transport, as it is far less sensitive to NBMPR (micromolar or weaker inhibition). Human ENT3, which was found to exhibit organellar localization, exhibits virtually no sensitivity to NBMPR. Human ENT4 was the last subtype to be identified—hENT4 is plasma membrane localized and completely NBMPR insensitive.

Human ENT subtypes exhibit sequence identities ranging from 25 to 50%, with hENT4 being the most genetically divergent member of the family. One shared feature of solute recognition among all human ENT subtypes is the preference for South (C2′-endo) ribose ring conformation for nucleoside substrate. Human ENTs generally exhibit broad tissue distributions, with subtypes 1-3 exhibiting substrate preference for both purines and pyrimidines. Interestingly, it has been shown human ENT1 and ENT2 can mediate low-affinity transport of nucleobases. Distinct from human ENT 1-3, human ENT4 can only transport adenosine, and no other nucleoside. Also known as the plasma membrane monoamine transporter (PMAT), hENT4 can also permeate monoamines, such as serotonin and dopamine and exhibits high levels of expression in heart and brain tissue.

Although human ENT1 and ENT2 are pH-independent uniporters, human ENT3 and ENT4 exhibit pH-dependent transport properties, and potential titratable residues have been reported for either subtype. Studies on human ENT3 have suggested that proton is not a co-transporting ion but regulates adenosine transport in human ENT3. Additional work is required to validate the conclusion of the role of pH in hENT3—however the potential proton in adenosine transport in hENT3 is interesting, as hENT3 functions in acidic organellar compartments. It is important to note that while adenosine transport by human ENT4 is pH-dependent, organic cation transport does not involve cotransport of ions or exhibit pH-dependence and is membrane potential dependent. Concerning the physiological role of human ENT4 in organic cation transport, it is a predominant neurotransmitter transporter present in astrocytes has been proposed to contribute to neurotransmitter clearance in the brain. The pH-dependent adenosine transport mediated by human ENT4 has been suggested to be physiologically relevant in the context of ischemia of the brain and heart but warrants further investigation.

### Inhibitors of Nucleoside Transport

Due to their high level of physiological involvement, inhibitors of specific human nucleoside transporters hold high pharmacological promise. One system with particular therapeutic relevance are human ENTs, which are arguably the main adenosine transporters in humans. Human ENT inhibitors block adenosine reuptake (adenosine reuptake inhibitors, termed AdoRIs) and lead to prolonged adenosine signaling. This pharmacological action is conceptually analogous to neurotransmitter sodium symporter (NSS) inhibitors—high affinity blockers of NSS activity include numerous clinically used antidepressants and mood modulators used to treat a wide variety of psychiatric disorders in humans. NSS block results in reuptake inhibition of neurotransmitters at chemical synapses, with leads to elevated concentrations of neurotransmitters at synapses for prolonged periods, thus potentiating neuronal signaling. Importantly, signaling potentiation only occurs in microenvironments in which signaling is actively occurring. This has advantages to directly targeting desired receptors, which requires systemic administration of receptor ligands. In the case of the adenosinergic system, such systemic distribution of synthetic agonists targeting adenosine GPCRs can lead to side-effects, owing off-tissue receptor activation and suboptimal receptor subtype selectivity. Potentiation of adenosine signaling by blocking ENT activity could potentially, in principle, lead to desired receptor activation with reduced off-target effects. Therefore, AdoRIs hold potential in the treatment of neurological disorders, pain, hypertension, heart disease and renal disorders—furthermore, several AdoRIs are currently used in the clinic as vasoactive agents.

AdoRIs are chemically diverse agents, with human ENT1 exhibiting the highest sensitivity to most AdoRIs. (FIG. 2). The hENT1 inhibitor NBMPR is a commonly tool used in nucleoside transport research, however it readily evolves the antimetabolite 6-mercaptopurine, which would prevent its clinical use. Other AdoRIs currently approved as human therapeutics include dipyridamole—an FDA approved coronary vasodilator that also inhibits phosphodiesterase-3 and dilazep—a low nanomolar hENT1 inhibitor used to treat renal IgA nephropathy. Additional classes of AdoRIs that may hold some unrealized therapeutic promise include draflazine/soluflazine (and derivatives)—highly potent AdoRIs with varying subtype and species selectivities, and rapadocin—a novel rapamycin-derivative which exhibits highly potent and subtype-specific inhibition of human ENT1 in a FKBP12-dependent manner.

### Compounds

The present disclosure provides, in part, novel compounds comprising, consisting of, or consisting essentially of novel equilibrative nucleoside transporter inhibitors and novel inhibitor scaffold inhibitors designed to sterically constrain dilazep for the use in the prevention and/or treatment of diseases, disorders and/or conditions associated ENT1 activity.

Accordingly, one aspect of the present disclosure provides a compound of formula (I) or a pharmaceutically acceptable salt thereof:

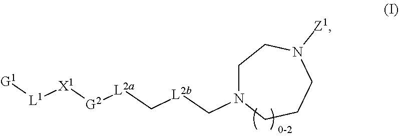

wherein:
G¹ is a 6- to 12-membered aryl or a 5- to 12-membered heteroaryl, wherein G¹ is optionally substituted with 1-4 R^{1x}, wherein, at each occurrence, R^{1x} is independently —NO₂, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, or —C₁₋₃alkylene-Q^{1a}; R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{1a}, at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L¹ is C₁₋₄alkylene or absent;
X¹ is O, S, or NH;
G² is phenylene, wherein G² is optionally substituted with 1-4 R^{2x}, wherein, at each occurrence, R^{2x} is independently halogen, C₁₋₆alkyl, C₁₋₆haloalkyl, C₂₋₆alkenyl, —OR^{2a}, —NO₂, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, or —C₁₋₃alkylene-Q^{2a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a};
R^{2d}, at each occurrence, is independently C₁₋₆alkyl, C₁₋₆haloalkyl, G^{2a}, or —C₁₋₃alkylene-G^{2a};
G^{2a}, at each occurrence, is independently a C₃₋₆cycloalkyl, a phenyl, a 4- to 6-membered heterocyclyl, or a 5- to 6-membered heteroaryl; wherein G^{2a} is optionally substituted with 1-5 substituents independently selected from the group consisting of halogen, oxo, C₁₋₄alkyl, —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{2a}, at each occurrence, is independently —OC₁₋₄alkyl, —OC₁₋₄haloalkyl, —OH, —NH₂, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L^{2a} is

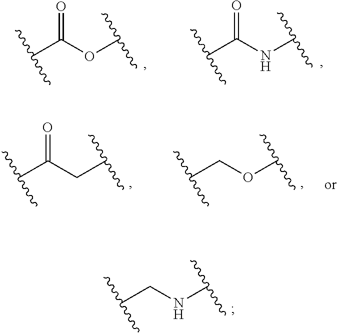

L^{2b} is C₁₋₃alkylene, C₂₋₃alkenylene, C₂₋₃alkynylene, or absent;
Z¹ is

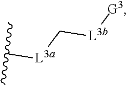

C₁₋₆alkyl, or C₃₋₆cycloalkyl;
L^{3a} is C₁₋₃alkylene, C₂₋₃alkenylene, C₂₋₃alkynylene, or absent; and
L^{3b} is

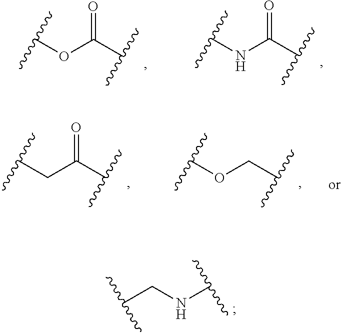

G³ is a 6- to 12-membered aryl or a 5- to 12-membered heteroaryl, wherein G³ is optionally substituted with 1-4 R^{3x}, wherein, at each occurrence, R^{3x} is independently halogen, C₁₋₆alkyl, C₁₋₆haloalkyl, C₂₋₆alkenyl, —OR^{3a}, —NO₂, —NR^{3a}R^{3b}, —SR^{3a}, —NR^{3a}C(O)R^{3c}, cyano, —C(O)OR^{3a}, —C(O)NR^{3a}R^{3b}, —C(O)R^{3c}, —SO₂R^{3d}, —SO₂NR^{3a}R^{3b}, G^{3a}, —C₁₋₃alkylene-G^{3a}, or —C₁₋₃alkylene-Q^{3a};
R^{3a}, R^{3b}, and R^{3c}, at each occurrence, are each independently hydrogen, C₁₋₆alkyl, C₁₋₆haloalkyl, G^{3a}, or —C₁₋₃alkylene-G^{3a};
R^{3d}, at each occurrence, is independently C₁₋₆alkyl, C₁₋₆haloalkyl, G^{3a}, or —C₁₋₃alkylene-G^{3a};
G^{3a}, at each occurrence, is independently a C₃₋₈cycloalkyl, a 4- to 12-membered heterocyclyl, a 6- to 12-membered aryl, or a 5- to 12-membered heteroaryl, wherein G^{3a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂; and
Q^{3a}, at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂.

In some instances, the compound may be a compound of formula (I-a):

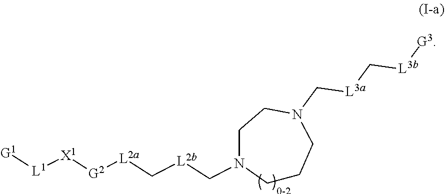

For example, the compound may be:

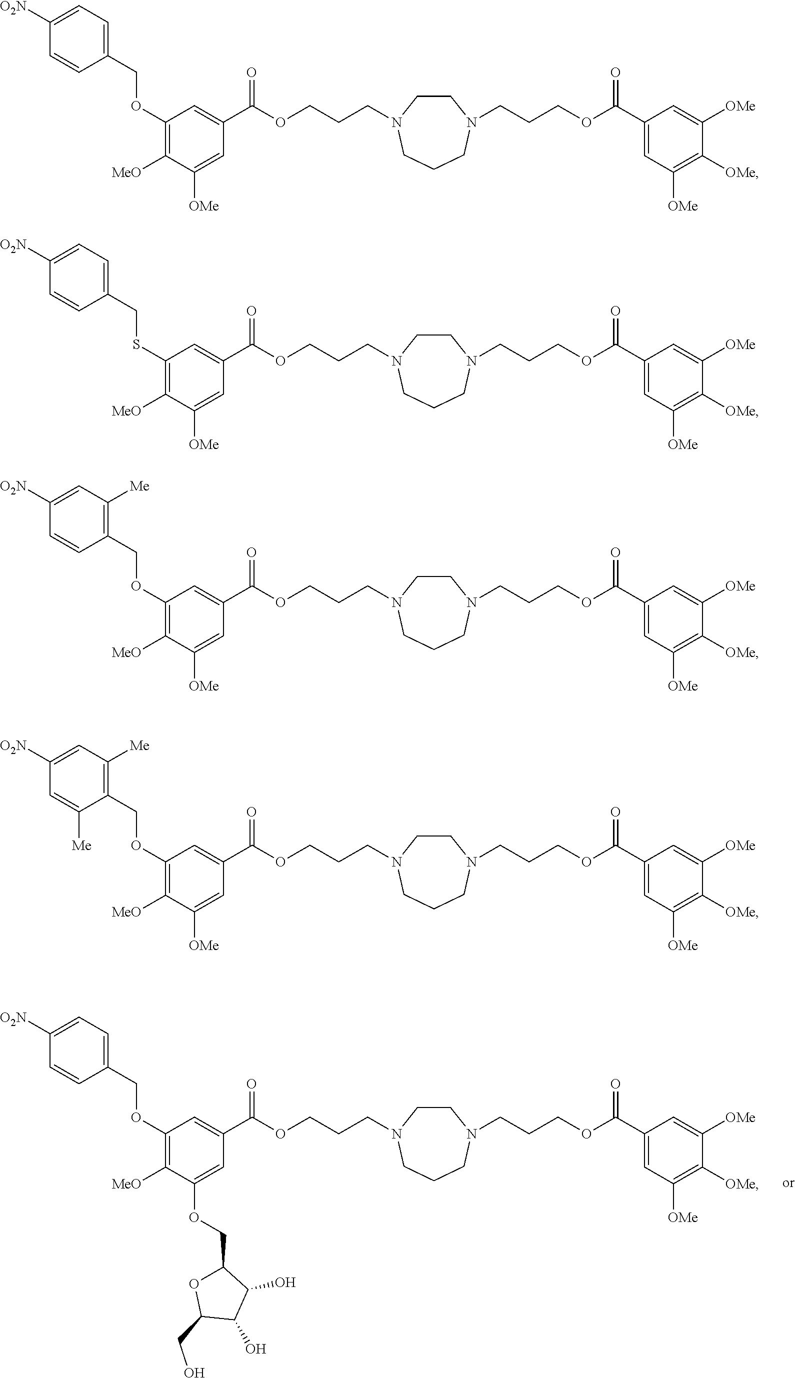

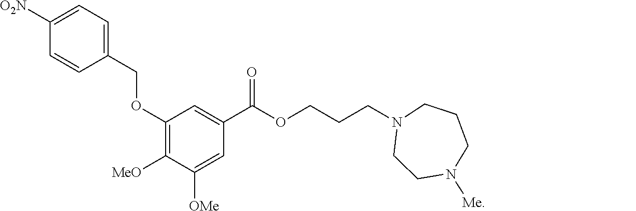 [SMILES: COc1cc(C(=O)OCCCN2CCCN(C)CC2)cc(OCc2ccc([N+](=O)[O-])cc2)c1OC]

Another aspect of the present disclosure provides a compound of formula (II) or a pharmaceutically acceptable salt thereof:

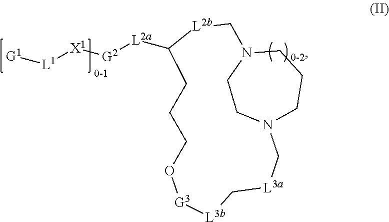

wherein:
G¹ is a 6- to 12-membered aryl or a 5- to 12-membered heteroaryl, wherein G¹ is optionally substituted with 1-4 R^{1x}, wherein, at each occurrence, R^{1x} is independently —NO₂, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, or —C₁₋₃alkylene-Q^{1a};
R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{1a}, at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄ alkyl, or —C(O)N(C₁₋₄alkyl)₂;
L¹ is C₁₋₄alkylene or absent;
X¹ is O, S, or NH;
when G¹-L¹-X¹ is present G² is phenylene;
when G¹-L¹-X¹ is absent G² is phenyl;
G² is optionally substituted with 1-4 R^{2x}, wherein, at each occurrence, R^{2x} is independently halogen, C₁₋₆alkyl, C₁₋₆haloalkyl, C₂₋₆alkenyl, —OR^{2a}, —NO₂, —NR^{2a}R^{2b}, —SR^{2a}, —NR^{2a}C(O)R^{2c}, cyano, —C(O)OR^{2a}, —C(O)R^{2c}, —C(O)NR^{2a}R^{2b}, —SO₂NR^{2a}R^{2b}, —SO₂R^{2d}, G^{2a}, —C₁₋₃alkylene-G^{2a}, or —C₁₋₃alkylene-Q^{2a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a};
R^{2d}, at each occurrence, is independently C₁₋₆alkyl, C₁₋₆haloalkyl, G^{2a}, or —C₁₋₃alkylene-G^{2a};
G^{2a}, at each occurrence, is independently a C₃₋₆cycloalkyl, a phenyl, a 4- to 6-membered heterocyclyl, or a 5- to 6-membered heteroaryl; wherein G^{2a} is optionally substituted with 1-5 substituents independently selected from the group consisting of halogen, oxo, C₁₋₄alkyl, —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{2a}, at each occurrence, is independently —OC₁₋₄alkyl, —OC₁₋₄haloalkyl, —OH, —NH₂, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L^{2a} is

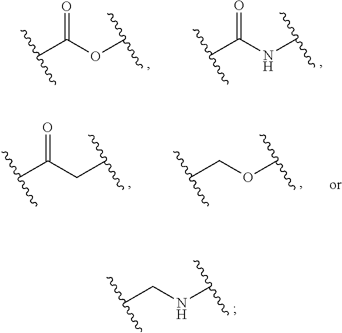

L^{2b} is C₁₋₃alkylene, C₂₋₃alkenylene, C₂₋₃alkynylene, or absent;
L^{3a} is C₁₋₃alkylene, C₂₋₃alkenylene, C₂₋₃alkynylene, or absent; and
L^{3b} is

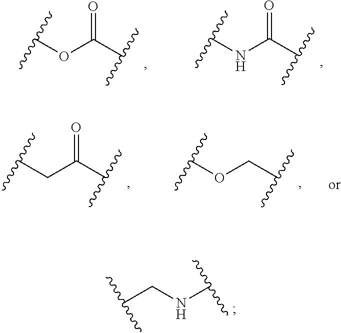

G³ is a 6- to 12-membered arylene or a 5- to 12-membered heteroarylene, wherein G³ is optionally substituted with 1-4 R^{3x}, wherein, at each occurrence, R^{3x} is independently halogen, C₁₋₆alkyl, C₁₋₆haloalkyl, C₂₋₆alkenyl, —OR^{3a}, —NO₂, —NR^{3a}R^{3b}, —SR^{3a}, —NR^{3a}C(O)R^{3c}, cyano, —C(O)OR^{3a}, —C(O)NR^{3a}R^{3b}, —C(O)R^{3c}, —SO₂R^{3d}, —SO₂NR^{3a}R^{3b}, G^{3a}, —C₁₋₃alkylene-G^{3a}, or —C₁₋₃alkylene-Q^{3a};
R^{3a}, R^{3b}, and R^{3c}, at each occurrence, are each independently hydrogen, C₁₋₆alkyl, C₁₋₆haloalkyl, G^{3a}, or —C₁₋₃alkylene-G^{3a};
R^{3d}, at each occurrence, is independently C₁₋₆alkyl, C₁₋₆haloalkyl, G^{3a}, or —C₁₋₃alkylene-G^{3a};
G^{3a}, at each occurrence, is independently a C₃₋₈cycloalkyl, a 4- to 12-membered heterocyclyl, a 6- to 12-membered aryl, or a 5- to 12-membered heteroaryl, wherein G^{3a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂; and
Q^{3a} at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂.

In some instances, the compound may be a compound of formula (II-a) or (II-b):

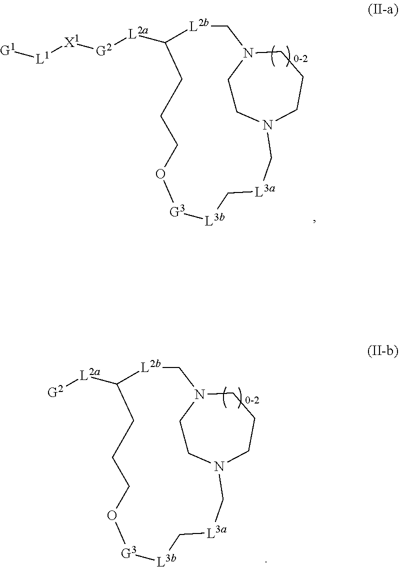

For example, the compound may be:

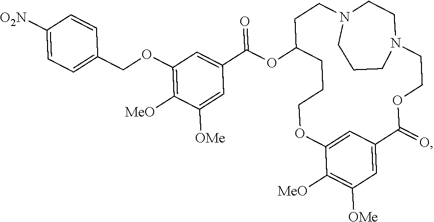 [SMILES: COc1cc2cc(c1OC)OCCCC(OC(=O)c1cc(OC)c(OC)c(OCc3ccc([N+](=O)[O-])cc3)c1)CCN1CCCN(CCOC2=O)CC1]

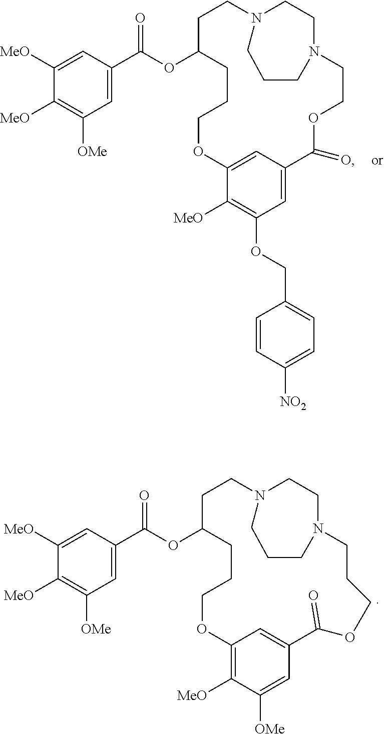 [SMILES: COc1cc(C(=O)OC2CCCOc3cc(cc(OCc4ccc([N+](=O)[O-])cc4)c3OC)C(=O)OCCN3CCCN(CC2)CC3)cc(OC)c1OC ; COc1cc(C(=O)OC2CCCOc3cc(cc(OC)c3OC)C(=O)OCCCN3CCCN(CC2)CC3)cc(OC)c1OC]

Another aspect of the present disclosure provides a compound of formula (III) or a pharmaceutically acceptable salt thereof:

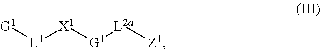

wherein:
G¹ is a 6- to 12-membered aryl or a 5- to 12-membered heteroaryl, wherein G¹ is optionally substituted with 1-4 R^{1x}, wherein, at each occurrence, R^{1x} is independently —NO₂, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, or —C₁₋₃alkylene-Q^{1a};
R¹, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{1a} at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L¹ is C₁₋₄alkylene or absent;
X¹ is O, S, or NH;
G² is phenylene, wherein G² is optionally substituted with 1-4 R^{2x}, wherein, at each occurrence, R^{2x} is independently halogen, C₁₋₆alkyl, C₁₋₆haloalkyl, C₂₋₆alkenyl, —OR^{2a}, —NO₂, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, or —C₁₋₃alkylene-Q^{2a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a};
R^{2d}, at each occurrence, is independently C₁₋₆alkyl, C₁₋₆haloalkyl, G^{2a}, or —C₁₋₃alkylene-G^{2a};
G^{2a}, at each occurrence, is independently a C₃₋₆cycloalkyl, a phenyl, a 4- to 6-membered heterocyclyl, or a 5- to 6-membered heteroaryl; wherein G^{2a} is optionally substituted with 1-5 substituents independently selected from the group consisting of halogen, oxo, C₁₋₄alkyl, —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{2a}, at each occurrence, is independently —OC₁₋₄alkyl, —OC₁₋₄haloalkyl, —OH, —NH₂, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L^{2a} is

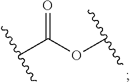 [SMILES: *OC(*)=O]

and
Z¹ is C₁₋₆alkyl, or C₃₋₆cycloalkyl.

In some instances, the compound may be:

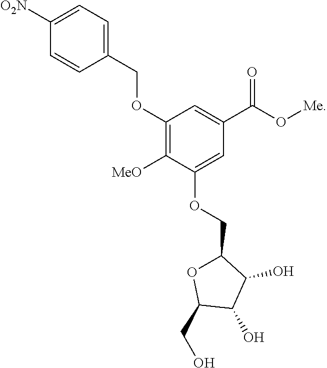 [SMILES: COC(=O)c1cc(OCc2ccc([N+](=O)[O-])cc2)c(OC)c(OC[C@@H]2O[C@H](CO)[C@@H](O)[C@H]2O)c1]

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

It should be understood that the compound may possess tautomeric forms, as well as geometric isomers, and that these also constitute an aspect of the invention.

In the compounds of formula (I), and any subformulas, any “hydrogen” or “H,” whether explicitly recited or implicit in the structure, encompasses hydrogen isotopes ¹H (protium) and ²H (deuterium).

The present disclosure also includes isotopically-labeled compounds (e.g., deuterium labeled), where an atom in the isotopically-labeled compound is specified as a particular isotope of the atom. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively.

Isotopically-enriched forms of compounds of formula (I), or any subformulas, may generally be prepared by conventional techniques known to those skilled in the art or by processes analogous to those described in the accompanying Examples using an appropriate isotopically-enriched reagent in place of a non-isotopically-enriched reagent. The extent of isotopic enrichment can be characterized as a percent incorporation of a particular isotope at an isotopically-labeled atom (e.g., % deuterium incorporation at a deuterium label).

### Pharmaceutical Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides, and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### General Synthesis of Compounds of Formulae (I)-(III)

### General Synthesis of Compounds of Formula (I)

Compounds of formula (I) or any of its subformulas may be synthesized as shown in the following schemes. In the following schemes G¹, L¹, X¹, G², L^{2b}, L^{3a}, L^{3b}, and G³ are as defined herein.

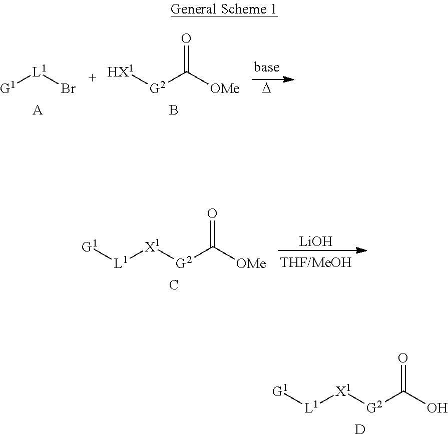

As shown in General Scheme 1, bromo-substituted compounds of formula A may be reacted with a —X¹H (e.g., —OH, —SH, —NH₃) substituted methyl ester of formula B under suitable reaction conditions to provide methyl ester intermediates of formula C. Intermediate C may be subsequently hydrolyzed under suitable conditions to provide carboxylic acids of formula D.

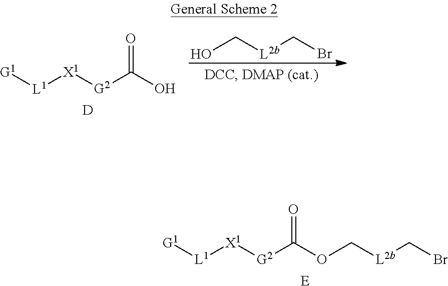

As shown in General Scheme 2, carboxylic acids of formula D may be reacted with a compound of formula Br—CH₂-L^{2b}-CH₂—OH under suitable Steglich Esterification conditions, e.g., in presence of dicyclohexylcarbodiimide (DCC) and 4-dimethylaminopyridine (DMAP), to provide bromoalkyl-substituted intermediates of formula E.

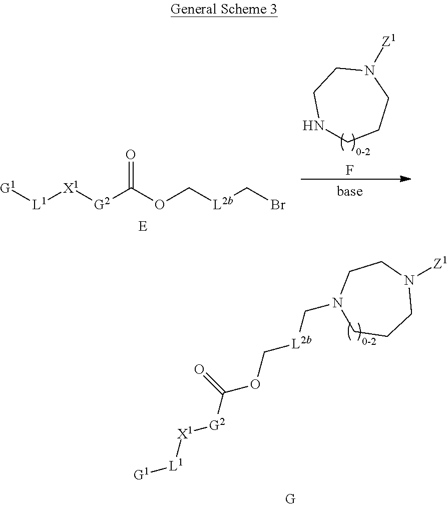

As shown in General Scheme 3, intermediate alkyl-bromide compounds of formula E may be reacted with a Z¹-substituted diazoheterocycle of formula F to provide compounds of formula G.

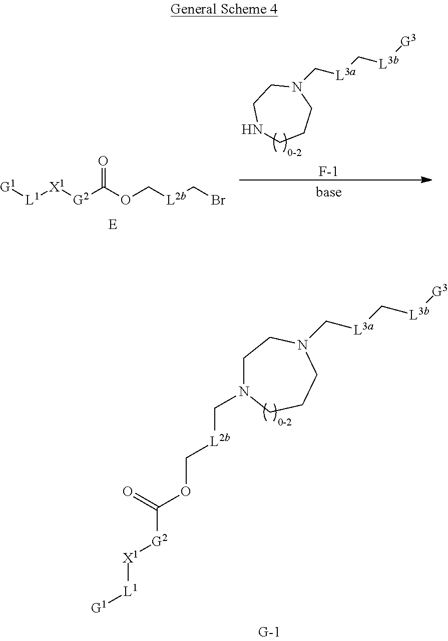

General Scheme 4 shows the preparation of example compounds of formula G-1 according to the general synthesis outlined in General Scheme 3 above.

### General Synthesis of Compounds of Formula (II)

Compounds of formula (II) or any of its subformulas may be synthesized as shown in the following schemes. In the following schemes G¹, L¹, X¹, G², L^{2b}, L^{3a}, L^{3b}, and G³ are as defined herein.

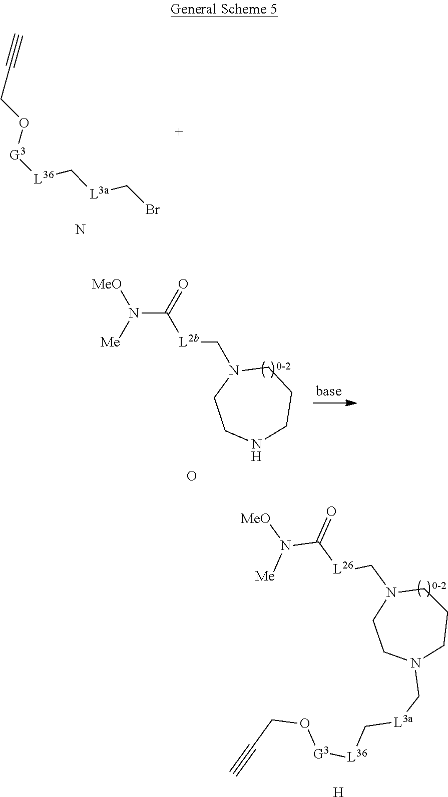

Prop-2-yn-1-yloxy-substituted alkyl-bromide compounds of formula N may be reacted with a Weinreb amide-substituted diazoheterocycle of formula O under suitable reaction conditions to provide bifunctional intermediates of formula H.

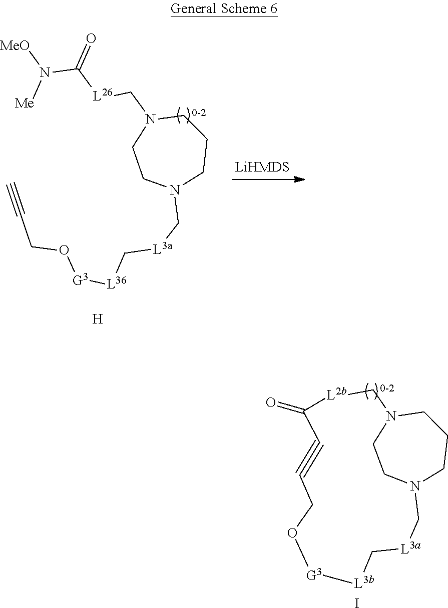

As shown in General Scheme 6, bifunctional intermediates of formula H may be cyclized under suitable conditions, e.g., in presence of organolithium reagent, to provide macrocyclic intermediates of formula I.

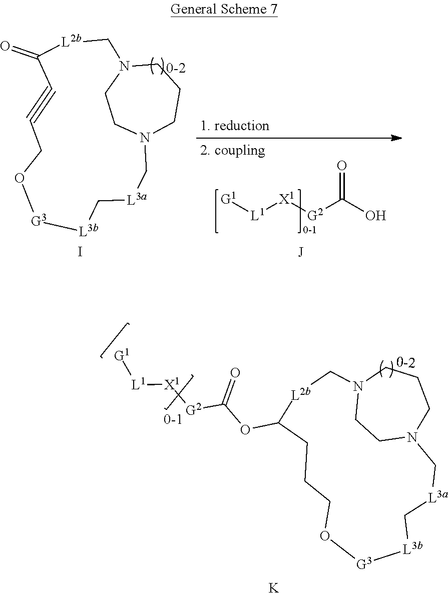

As shown in General Scheme 7, macrocyclic intermediates of formula I may be subjected to suitable reduction conditions, followed by coupling with a carboxylic acid of formula J under suitable coupling conditions to provide macrocyclic compounds of formula K.

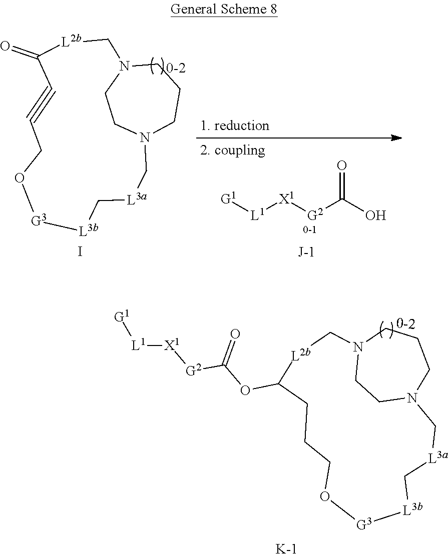

General Scheme 8 shows the preparation of example macrocyclic compounds of formula K-1 from intermediates I and J-1, according to the general synthesis outlined in General Scheme 7 above.

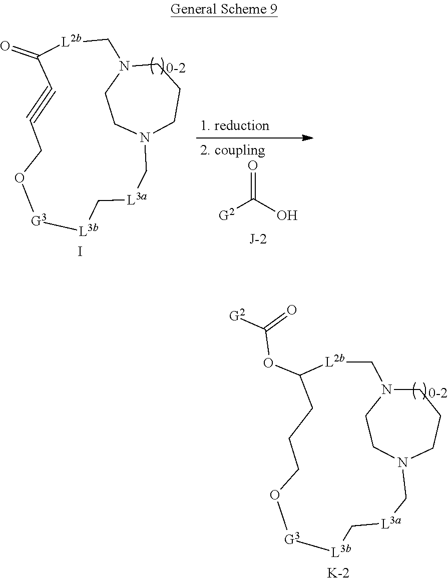

General Scheme 9 shows the preparation of example macrocyclic compounds of formula K-2 from intermediates I and J-2, according to the general synthesis outlined in General Scheme 7 above.

### General Synthesis of Compounds of Formula (III)

Compounds of formula (III) or any of its subformulas may be synthesized as shown in the following schemes.

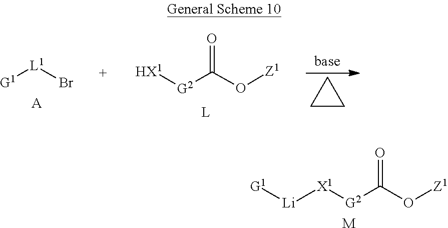

As shown in General Scheme 10, bromo-substituted compounds of formula A may be reacted with a —X¹H (e.g., —OH, —SH, —NH₃) substituted ester compound of formula L under suitable reaction conditions to provide compounds of formula M.

The compounds and intermediates described above may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in Vogel's Textbook of Practical Organic Chemistry”, 5^{th} ed. (1989), by Furniss, Hannaford, Smith, and Tatchell, Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the particular reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature.

Starting materials, if not commercially available, can be prepared by procedures selected from standard organic chemical techniques, techniques that are analogous to the synthesis of known, structurally similar compounds, or techniques that are analogous to the above-described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in P G M Wuts and T W Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### Pharmaceutical Compositions

In another aspect, the present disclosure provides compositions comprising one or more of compounds as described herein and an appropriate carrier, excipient, or diluent. The exact nature of the carrier, excipient or diluent will depend upon the desired use for the composition and may range from being suitable or acceptable for veterinary uses to being suitable or acceptable for human use. The composition may optionally include one or more additional compounds.

When used to treat or prevent a disease, such as a bacterial infection, the compounds described herein may be administered singly, as mixtures of one or more compounds or in mixture or combination with other agents (e.g., therapeutic agents) useful for treating such diseases and/or the symptoms associated with such diseases, such as pain. Such agents may include, but are not limited to, NSAIDS, anti-inflammatory compounds, analgesics, and Cox2 inhibitors to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients, or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal, or intraperitoneal injection, as well as those designed for transdermal, transmucosal oral or pulmonary administration.

Useful injectable preparations include sterile suspensions, solutions, or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films, or enteric coatings.

Liquid preparations for oral administration may take the form of, for example, elixirs, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC50 of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### Methods

The compounds and pharmaceutical compositions provided herein have many uses in the treatment and prevention of diseases, disorders and/or conditions associated with ENT1, such as pain.

Hence, another aspect of the present disclosure provides a method of inhibiting ENT1 in a cell, the method comprising, consisting of, or consisting essentially of contacting the cell with a compound, or a pharmaceutical composition thereof, as provided herein such that ENT1 is inhibited in the cell.

Another aspect of the present disclosure provides a method of inhibiting ENT1 in a subject, the method comprising, consisting of, or consisting essentially of contacting the cell with a compound, or a pharmaceutical composition thereof, as provided herein such that ENT1 is inhibited in the subject.

Another aspect of the present disclosure provides a method of treating and/or preventing pain in a subject, the method comprising, consisting of, or consisting essentially of administering to the subject a therapeutically effective amount of a compound as provided herein, or a pharmaceutical composition thereof, such that the pain is treated and/or prevented in the subject.

In one embodiment, the composition is administered intrathecally. In another embodiment, pain comprises neuropathic pain.

In another embodiment, the method further comprises administering to the subject a therapeutically effective amount of at least one additional therapeutic agent. In one embodiment, the at least one additional therapeutic agent is administered before the compound or pharmaceutical composition thereof. In another embodiment, the at least one additional therapeutic agent is administered concurrently with the compound or pharmaceutical composition thereof. In yet another embodiment, the at least one additional therapeutic agent is administered after the compound or pharmaceutical composition thereof.

### Kits

The present disclosure further provides kits comprising the compounds and/or pharmaceutical compositions provided herein and for carrying out the subject methods as provided herein. For example, in one embodiment, a subject kit may comprise, consist of, or consist essentially of: (i) a compound as provided herein; (ii) a CellREADR system as provided herein; and/or (ii) pharmaceutical compositions as provided herein.

In other embodiments, a kit may further include other components. Such components may be provided individually or in combination and may provide in any suitable container such as a vial, a bottle, or a tube. Examples of such components include, but are not limited to one or more additional reagents, such as one or more dilution buffers; one or more reconstitution solutions; one or more wash buffers; one or more storage buffers, one or more control reagents and the like. Components (e.g., reagents) may also be provided in a form that is usable in a particular assay, or in a form that requires addition of one or more other components before use (e.g., in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (e.g., associated with the packaging or subpackaging) etc. In other embodiments, the instructions are present as an electronic storage data file present on a suitable computer readable storage medium, e.g., CD-ROM, diskette, flash drive, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed.

Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A compound of formula (I), or a pharmaceutically acceptable salt thereof:

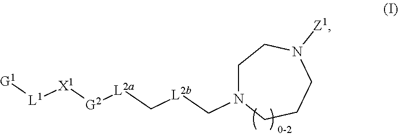

wherein:
G¹ is a 6- to 12-membered aryl or a 5- to 12-membered heteroaryl, wherein G¹ is optionally substituted with 1-4 R^{1x}, wherein, at each occurrence, R^{1x} is independently —NO₂, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, or —C₁₋₃alkylene-Q^{1a};
R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{1a} at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L¹ is C₁₋₄alkylene or absent;
X¹ is O, S, or NH;
G² is phenylene, wherein G² is optionally substituted with 1-4 R^{2x}, wherein, at each occurrence, R^{2x} is independently halogen, C₁₋₆alkyl, C₁₋₆haloalkyl, C₂₋₆alkenyl, —OR^{2a}, —NO₂, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, or —C₁₋₃alkylene-Q^{2a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a};
R^{2d}, at each occurrence, is independently C₁₋₆alkyl, C₁₋₆haloalkyl, G^{2a}, or —C₁₋₃alkylene-G^{2a};
G^{2a}, at each occurrence, is independently a C₃₋₆cycloalkyl, a phenyl, a 4- to 6-membered heterocyclyl, or a 5- to 6-membered heteroaryl; wherein G^{2a} is optionally substituted with 1-5 substituents independently selected from the group consisting of halogen, oxo, C₁₋₄alkyl, —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{2a}, at each occurrence, is independently —OC₁₋₄alkyl, —OC₁₋₄haloalkyl, —OH, —NH₂, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L^{2a} is

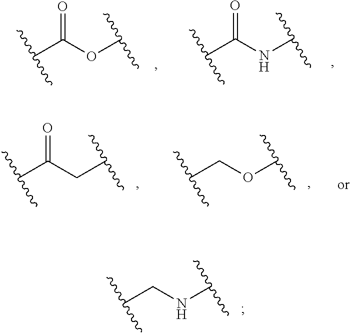

L^{2b} is C₁₋₃alkylene, C₂₋₃alkenylene, C₂₋₃alkynylene, or absent;
Z¹ is

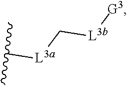

C₁₋₆alkyl, or C₃₋₆cycloalkyl;
L^{3a} is C₁₋₃alkylene, C₂₋₃alkenylene, C₂₋₃alkynylene, or absent; and
L^{3b} is

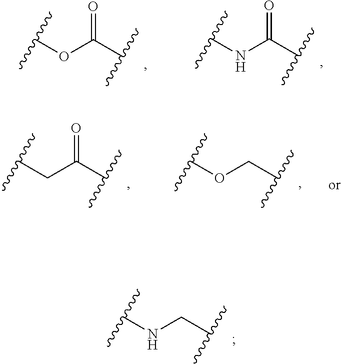

G³ is a 6- to 12-membered aryl or a 5- to 12-membered heteroaryl, wherein G³ is optionally substituted with 1-4 R^{3x}, wherein, at each occurrence, R^{3x} is independently halogen, C₁₋₆alkyl, C₁₋₆haloalkyl, C₂₋₆alkenyl, —OR^{3a}, —NO₂, —NR^{3a}R^{3b}, —SR^{3a}, —NR^{3a}C(O)R^{3c}, cyano, —C(O)OR^{3a}, —C(O)NR^{3a}R^{3b}, —C(O)R^{3c}, —SO₂R^{3d}, —SO₂NR^{3a}R^{3b}, G^{3a}, —C₁₋₃alkylene-G^{3a}, or —C₁₋₃alkylene-Q^{3a};
R^{3a}, R^{3b}, and R^{3c}, at each occurrence, are each independently hydrogen, C₁₋₆alkyl, C₁₋₆haloalkyl, G^{3a}, or —C₁₋₃alkylene-G^{3a};
R^{3d}, at each occurrence, is independently C₁₋₆alkyl, C₁₋₆haloalkyl, G^{3a}, or —C₁₋₃alkylene-G^{3a};
G^{3a}, at each occurrence, is independently a C₃₋₈cycloalkyl, a 4- to 12-membered heterocyclyl, a 6- to 12-membered aryl, or a 5- to 12-membered heteroaryl, wherein G^{3a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂; and
Q^{3a} at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄ alkyl, or —C(O)N(C₁₋₄alkyl)₂.
Clause 2. The compound of clause 1, or a pharmaceutically acceptable salt thereof, wherein the compound is a compound of formula (I-a):

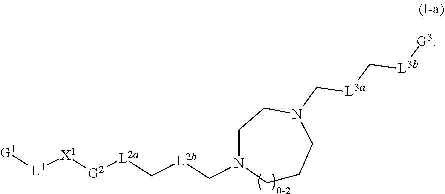

Clause 3. The compound of clause 1 or 2, or a pharmaceutically acceptable salt thereof, wherein G¹ is phenyl.
Clause 4. The compound of any one of clauses 1-3, or a pharmaceutically acceptable salt thereof, wherein G¹ is substituted with 1-3 R^{1x}, wherein, at each occurrence, R^{1x} is independently —NO₂ or C₁₋₄alkyl.
Clause 5. The compound of any one of clauses 1-4, or a pharmaceutically acceptable salt thereof, wherein G¹ is

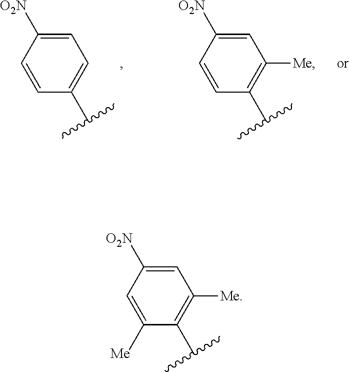 [SMILES: *c1ccc([N+](=O)[O-])cc1 ; *c1ccc([N+](=O)[O-])cc1C ; *c1c(C)cc([N+](=O)[O-])cc1C]

Clause 6. The compound of any one of clauses 1-5, or a pharmaceutically acceptable salt thereof, wherein L¹ is C₁₋₃alkylene.
Clause 7. The compound of any one of clauses 1-6, or a pharmaceutically acceptable salt thereof, wherein X¹ is O or S.
Clause 8. The compound of any one of clauses 1-7, or a pharmaceutically acceptable salt thereof, wherein G² is substituted with 1-3 R^{2x}, wherein, at each occurrence, R^{2x} is —OR^{2a}.
Clause 9. The compound of clause 8, or a pharmaceutically acceptable salt thereof, wherein R^{2a} is C₁₋₄alkyl, cyclopropyl, or —C₁₋₃alkylene-G^{2a}.
Clause 10. The compound of clause 8 or 9, or a pharmaceutically acceptable salt thereof, wherein at least one R^{2a} is methyl, ethyl, or isopropyl.
Clause 11. The compound of clause 9, or a pharmaceutically acceptable salt thereof, wherein G^{2a} is

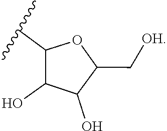 [SMILES: *C1OC(CO)C(O)C1O]

Clause 12. The compound of any one of clauses 1-11, or a pharmaceutically acceptable salt thereof, wherein L^{2a} is

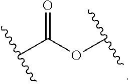 [SMILES: *OC(*)=O]

Clause 13. The compound of any one of clauses 1-12, or a pharmaceutically acceptable salt thereof, wherein L^{2b} is C₁₋₃alkylene.
Clause 14. The compound of any one of clauses 1-13, or a pharmaceutically acceptable salt thereof, wherein

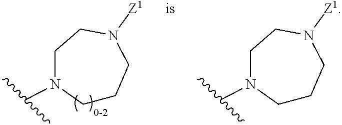

Clause 15. The compound of any one of clauses 1-14, or a pharmaceutically acceptable salt thereof, wherein Z¹ is

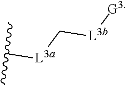

Clause 16. The compound of any one of clauses 1-15, or a pharmaceutically acceptable salt thereof, wherein L^{3a} is C₁₋₃alkylene.
Clause 17. The compound of any one of clauses 1-16, or a pharmaceutically acceptable salt thereof, wherein L^{3b} is

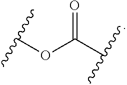 [SMILES: *OC(*)=O]

Clause 18. The compound of any one of clauses 1-17, or a pharmaceutically acceptable salt thereof, wherein G³ is phenyl.
Clause 19. The compound of any one of clauses 1-18, or a pharmaceutically acceptable salt thereof, wherein G³ is substituted with 1-3 R^{3x}, wherein R^{3x} is —OR^{3a}.
Clause 20. The compound of clause 19, or a pharmaceutically acceptable salt thereof, wherein R^{3a} is C₁₋₄alkyl or cyclopropyl.
Clause 21. The compound of any one of clauses 1-14, or a pharmaceutically acceptable salt thereof, wherein Z¹ is C₁₋₄alkyl or cyclopropyl.
Clause 22. The compound of clause 1, or a pharmaceutically acceptable salt thereof, wherein the compound is:

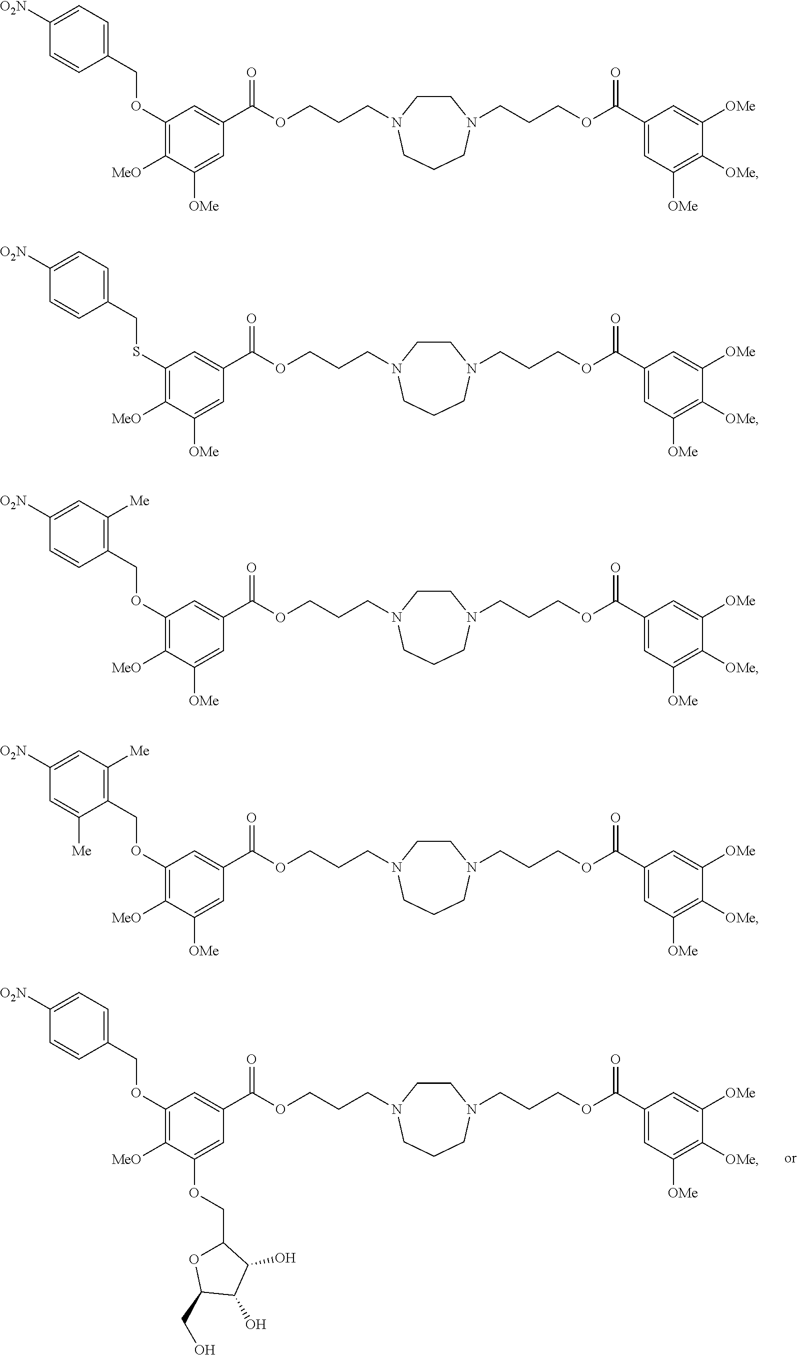 [SMILES: COc1cc(C(=O)OCCCN2CCCN(CCCOC(=O)c3cc(OC)c(OC)c(OCc4ccc([N+](=O)[O-])cc4)c3)CC2)cc(OC)c1OC ; COc1cc(C(=O)OCCCN2CCCN(CCCOC(=O)c3cc(OC)c(OC)c(SCc4ccc([N+](=O)[O-])cc4)c3)CC2)cc(OC)c1OC ; COc1cc(C(=O)OCCCN2CCCN(CCCOC(=O)c3cc(OC)c(OC)c(OCc4ccc([N+](=O)[O-])cc4C)c3)CC2)cc(OC)c1OC ; COc1cc(C(=O)OCCCN2CCCN(CCCOC(=O)c3cc(OC)c(OC)c(OCc4c(C)cc([N+](=O)[O-])cc4C)c3)CC2)cc(OC)c1OC ; COc1cc(C(=O)OCCCN2CCCN(CCCOC(=O)c3cc(OCc4ccc([N+](=O)[O-])cc4)c(OC)c(OCC4O[C@H](CO)[C@@H](O)[C@H]4O)c3)CC2)cc(OC)c1OC ; "or"]

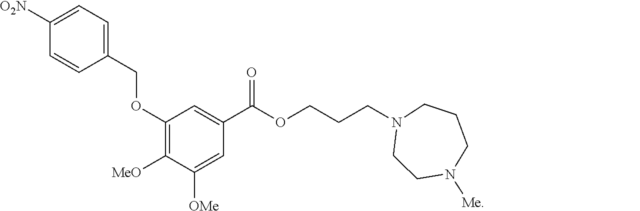 [SMILES: COc1cc(C(=O)OCCCN2CCCN(C)CC2)cc(OCc2ccc([N+](=O)[O-])cc2)c1OC]

Clause 23. A pharmaceutical composition comprising the compound of any one of clauses 1-22, or a pharmaceutically acceptable salt thereof, and a pharmaceutically acceptable carrier.
Clause 24. A method of treating neuropathic pain, the method comprising administering to a subject in need thereof a therapeutically effective amount of the compound of any one of clauses 1-22, or a pharmaceutically acceptable salt thereof, or the pharmaceutical composition of clause 23.
Clause 25. A method of inhibiting an equilibrative nucleoside transporter (ENT), the method comprising administering to a subject in need thereof a therapeutically effective amount of the compound of any one of clauses 1-22, or a pharmaceutically acceptable salt thereof, or the pharmaceutical composition of clause 23.
Clause 26. The method of clause 25, wherein the equilibrative nucleoside transporter is equilibrative nucleoside transporter 1 (ENT1).
Clause 27. A compound of formula (II), or a pharmaceutically acceptable salt thereof:

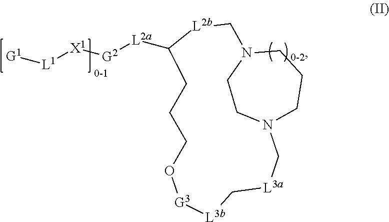

wherein:
G¹ is a 6- to 12-membered aryl or a 5- to 12-membered heteroaryl, wherein G¹ is optionally substituted with 1-4 R^{1x}, wherein, at each occurrence, R^{1x} is independently —NO₂, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, or —C₁₋₃alkylene-Q^{1a};
R¹, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{1a} at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L¹ is C₁₋₄alkylene or absent;
X¹ is O, S, or NH;
when G¹-L¹-X¹ is present G² is phenylene;
when G¹-L¹-X¹ is absent G² is phenyl; G² is optionally substituted with 1-4 R^{2x}, wherein, at each occurrence, R^{2x} is independently halogen, C₁₋₆alkyl, C₁₋₆haloalkyl, C₂₋₆alkenyl, —OR^{2a}, —NO₂, —NR^{2a}R^{2b}, —SR^{2a}, —NR^{2a}C(O)R^{2c}, cyano, —C(O)OR^{2a}, —C(O)R^{2c}, —C(O)NR^{2a}R^{2b}, —SO₂NR^{2a}R^{2b}, —SO₂R^{2d}, G^{2a}, —C₁₋₃alkylene-G^{2a}, or —C₁₋₃alkylene-Q^{2a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a};
R^{2d}, at each occurrence, is independently C₁₋₆alkyl, C₁₋₆haloalkyl, G^{2a}, or —C₁₋₃alkylene-G^{2a};
G^{2a}, at each occurrence, is independently a C₃₋₆cycloalkyl, a phenyl, a 4- to 6-membered heterocyclyl, or a 5- to 6-membered heteroaryl; wherein G^{2a} is optionally substituted with 1-5 substituents independently selected from the group consisting of halogen, oxo, C₁₋₄alkyl, —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{2a}, at each occurrence, is independently —OC₁₋₄alkyl, —OC₁₋₄haloalkyl, —OH, —NH₂, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L^{2a} is

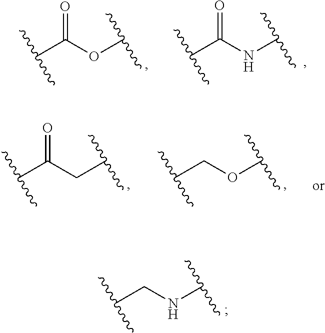

L^{2b} is C₁₋₃alkylene, C₂₋₃alkenylene, C₂₋₃alkynylene, or absent;
L^{3a} is C₁₋₃alkylene, C₂₋₃alkenylene, C₂₋₃alkynylene, or absent; and
L^{3b} is

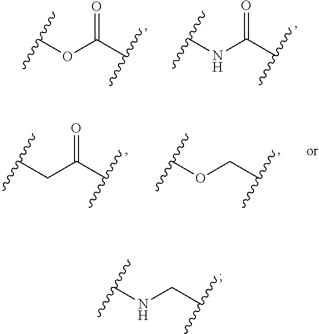

G³ is a 6- to 12-membered arylene or a 5- to 12-membered heteroarylene, wherein G³ is optionally substituted with 1-4 R^{3x}, wherein, at each occurrence, R^{3x} is independently halogen, C₁₋₆alkyl, C₁₋₆haloalkyl, C₂₋₆alkenyl, —OR^{3a}, —NO₂, —NR^{3a}R^{3b}, —SR^{3a}, —NR^{3a}C(O)R^{3c}, cyano, —C(O)OR^{3a}, —C(O)NR^{3a}R^{3b}, —C(O)R^{3c}, —SO₂R^{3d}, —SO₂NR^{3a}R^{3b}, G^{3a}, —C₁₋₃alkylene-G^{3a}, or —C₁₋₃alkylene-Q^{3a};
R^{3a}, R^{3b}, and R^{3c}, at each occurrence, are each independently hydrogen, C₁₋₆alkyl, C₁₋₆haloalkyl, G^{3a}, or —C₁₋₃alkylene-G^{3a};
R^{3d}, at each occurrence, is independently C₁₋₆alkyl, C₁₋₆haloalkyl, G^{3a}, or —C₁₋₃alkylene-G^{3a};
G^{3a}, at each occurrence, is independently a C₃₋₈cycloalkyl, a 4- to 12-membered heterocyclyl, a 6- to 12-membered aryl, or a 5- to 12-membered heteroaryl, wherein G^{3a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂; and
Q^{3a}, at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂.
Clause 28. The compound of clause 27, or a pharmaceutically acceptable salt thereof, wherein or a pharmaceutically acceptable salt thereof, wherein the compound is a compound of formula (II-a) or (II-b):

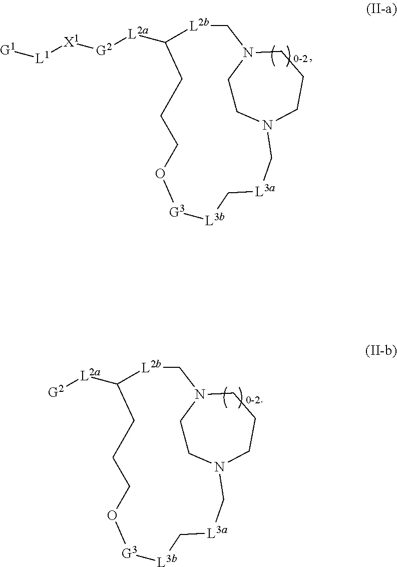

Clause 29. The compound of clause 27 or 28, or a pharmaceutically acceptable salt thereof, wherein G¹ is phenyl.
Clause 30. The compound of any one of clauses 27-29, or a pharmaceutically acceptable salt thereof, wherein G¹ is substituted with 1-3 R^{1x}, wherein, at each occurrence, R^{1x} is independently —NO₂ or C₁₋₄alkyl.
Clause 31. The compound of any one of clauses 27-30, or a pharmaceutically acceptable salt thereof, wherein L¹ is C₁₋₄alkylene and X¹ is O or S.
Clause 32. The compound of any one of clauses 27-31, or a pharmaceutically acceptable salt thereof, wherein G² is substituted with 1-3 R^{2x}, wherein, at each occurrence, R^{2x} is —OR^{2a}.
Clause 33. The compound of any one of clauses 27-32, or a pharmaceutically acceptable salt thereof, wherein L^{2a} is

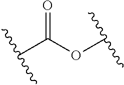 [SMILES: *OC(*)=O]

and L^{2b} is C₁₋₃alkylene.
Clause 34. The compound of any one of clauses 27-33, or a pharmaceutically acceptable salt thereof, wherein

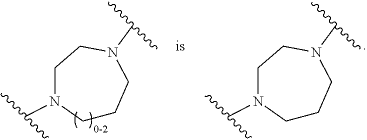

Clause 35. The compound of any one of clauses 27-34, or a pharmaceutically acceptable salt thereof, wherein L^{3a} is C₁₋₃alkylene.
Clause 36. The compound of any one of clauses 27-35, or a pharmaceutically acceptable salt thereof, wherein L^{3b} is

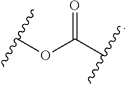 [SMILES: *OC(*)=O]

Clause 37. The compound of any one of clauses 27-36, or a pharmaceutically acceptable salt thereof, wherein G³ is phenylene.
Clause 38. The compound of any one of clauses 27-37, or a pharmaceutically acceptable salt thereof, wherein G³ is substituted with 1-3 R^{3x}, wherein R^{3x} is —OR^{3a}.
Clause 39. The compound of clause 27, or a pharmaceutically acceptable salt thereof, wherein the compound is:

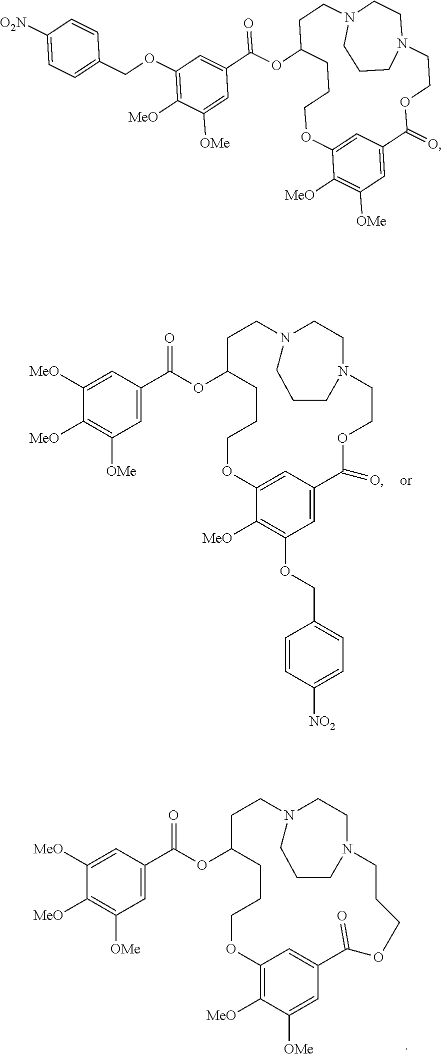 [SMILES: COc1cc2cc(c1OC)OCCCC(OC(=O)c1cc(OC)c(OC)c(OCc3ccc([N+](=O)[O-])cc3)c1)CCN1CCCN(CCOC2=O)CC1 ; COc1cc(C(=O)OC2CCCOc3cc(cc(OCc4ccc([N+](=O)[O-])cc4)c3OC)C(=O)OCCN3CCCN(CC2)CC3)cc(OC)c1OC ; COc1cc(C(=O)OC2CCCOc3cc(cc(OC)c3OC)C(=O)OCCCN3CCCN(CC2)CC3)cc(OC)c1OC]

Clause 40. A pharmaceutical composition comprising the compound of any one of clauses 27-39, or a pharmaceutically acceptable salt thereof, and a pharmaceutically acceptable carrier.
Clause 41. A method of treating neuropathic pain, the method comprising administering to a subject in need thereof a therapeutically effective amount of the compound of any one of clauses 27-39, or a pharmaceutically acceptable salt thereof, or the pharmaceutical composition of clause 40.
Clause 42. A method of inhibiting an equilibrative nucleoside transporter (ENT), the method comprising administering to a subject in need thereof a therapeutically effective amount of the compound of any one of clauses 27-39, or a pharmaceutically acceptable salt thereof, or the pharmaceutical composition of clause 40.
Clause 43. A compound of formula (III), or a pharmaceutically acceptable salt thereof:

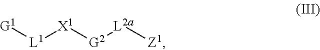

wherein:
G¹ is a 6- to 12-membered aryl or a 5- to 12-membered heteroaryl, wherein G¹ is optionally substituted with 1-4 R^{1x}, wherein, at each occurrence, R^{1x} is independently —NO₂, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, or —C₁₋₃alkylene-Q^{1a};
R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{1a} at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L¹ is C₁₋₄alkylene or absent;
X¹ is O, S, or NH;
G² is phenylene, wherein G² is optionally substituted with 1-4 R^{2x}, wherein, at each occurrence, R^{2x} is independently halogen, C₁₋₆alkyl, C₁₋₆haloalkyl, C₂₋₆alkenyl, —OR^{2a}, —NO₂, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, or —C₁₋₃alkylene-Q^{2a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a};
R^{2d}, at each occurrence, is independently C₁₋₆alkyl, C₁₋₆haloalkyl, G^{2a}, or —C₁₋₃alkylene-G^{2a};
G^{2a}, at each occurrence, is independently a C₃₋₆cycloalkyl, a phenyl, a 4- to 6-membered heterocyclyl, or a 5- to 6-membered heteroaryl; wherein G^{2a} is optionally substituted with 1-5 substituents independently selected from the group consisting of halogen, oxo, C₁₋₄alkyl, —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{2a}, at each occurrence, is independently —OC₁₋₄alkyl, —OC₁₋₄haloalkyl, —OH, —NH₂, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L^{2a} is

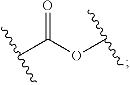 [SMILES: *OC(*)=O]

and
Z¹ is C₁₋₆alkyl, or C₃₋₆cycloalkyl.
Clause 44. The compound of clause 43, or a pharmaceutically acceptable salt thereof, wherein G¹ is

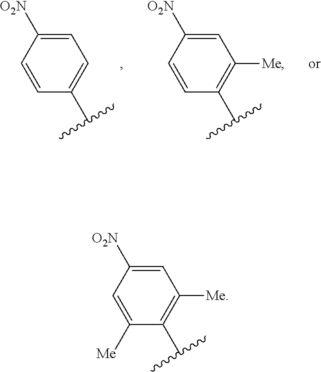 [SMILES: *c1ccc([N+](=O)[O-])cc1 ; *c1ccc([N+](=O)[O-])cc1C ; *c1c(C)cc([N+](=O)[O-])cc1C]

Clause 45. The compound of clause 43 or 44, or a pharmaceutically acceptable salt thereof, wherein G² is

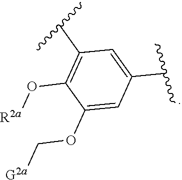

Clause 46. The compound of any one of clauses 43-45, or a pharmaceutically acceptable salt thereof, wherein the compound is:

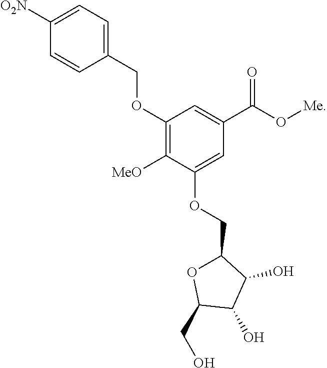 [SMILES: COC(=O)c1cc(OCc2ccc([N+](=O)[O-])cc2)c(OC)c(OC[C@@H]2O[C@H](CO)[C@@H](O)[C@H]2O)c1]

Clause 47. A pharmaceutical composition comprising the compound of any one of clauses 43-46, or a pharmaceutically acceptable salt thereof, and a pharmaceutically acceptable carrier.
Clause 48. A method of treating neuropathic pain, the method comprising administering to a subject in need thereof a therapeutically effective amount of the compound of any one of clauses 43-46, or a pharmaceutically acceptable salt thereof, or the pharmaceutical composition of clause 47.
Clause 49. A method of inhibiting an equilibrative nucleoside transporter (ENT), the method comprising administering to a subject in need thereof a therapeutically effective amount of the compound of any one of clauses 43-46, or a pharmaceutically acceptable salt thereof, or the pharmaceutical composition of clause 47.
Clause 50. The method of clause 49, wherein the equilibrative nucleoside transporter is equilibrative nucleoside transporter 1 (ENT1).

### EXAMPLES

### Example 1

Structures of Human ENT1 Complexed with Adenosine Reuptake Inhibitors Prior to the work on this transporter, no experimental structure was available for any ENT family member. The first molecular cloning of ENT genes led to the postulation that they share a common ancestor with MFS transporters based on sequence analysis—however, it was unclear at the time due to the fact extremely low sequence identities (<15%) are apparent between members of the ENT family and MFS members. Despite this uncertainty, experimental structures of MFS members E. coli lactose permease (LacY) and fucose permease (FucP) were used as a templates for ab initio homolog modeling of the ENT structures. These studies focused on the L. donovani nucleoside transporter 1.1 (LdNT1.1) and resulted in models constructed in inward and outward facing conformations, which combined with functional experiments uncovered general features of the ENT fold. Additionally, ENT structure predictions have been performed with de novo prediction pipelines. However, these predictions were of low quality, due to the limitations of templated based homolog modeling (little to no sequence similarity with templates of known structure) or the challenges of membrane protein structure prediction using de novo, template-free methods. Therefore, a goal was to solve the first experimental structures of human ENT1. Such data would enable a comprehensive understanding of nucleoside recognition, inhibition by AdoRIs and the transport mechanism exhibited by ENTs.

### Biochemical Optimization of Human ENT1

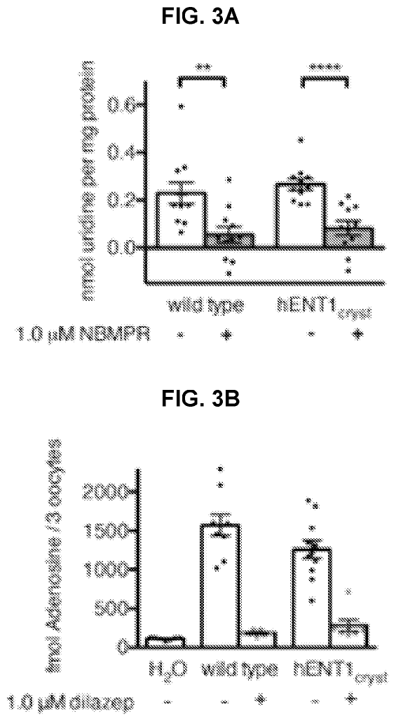

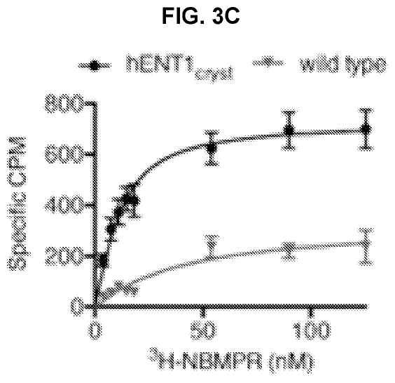

A substantial challenge in obtaining crystal structures of human ENT1 had been the biochemical instability of detergent purified protein. Therefore, efforts were undertaken to stabilize human ENT1, in order to increase the likelihood of crystallization and subsequent structure determination by X-ray crystallography. Wild-type human ENT1 expresses well in HEK293T cells and exhibits monodisperse behavior during fluorescence size exclusion chromatography (FSEC) analysis under normal conditions (FIG. 3A). However, the detergent extracted protein is highly heat labile, as evident by an apparent thermal melting temperature (Tₘ) of ˜40° C. as measured by FSEC analysis (FIG. 3A). This thermal lability is substantially reduced by the inclusion of the AdoRI dilazep during sample heating, indicating the potent AdoRI binds and stabilizes detergent solubilized hENT1 in vitro (FIG. 3B).

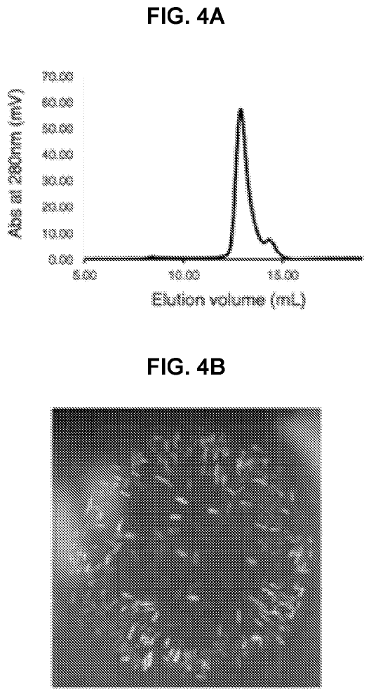

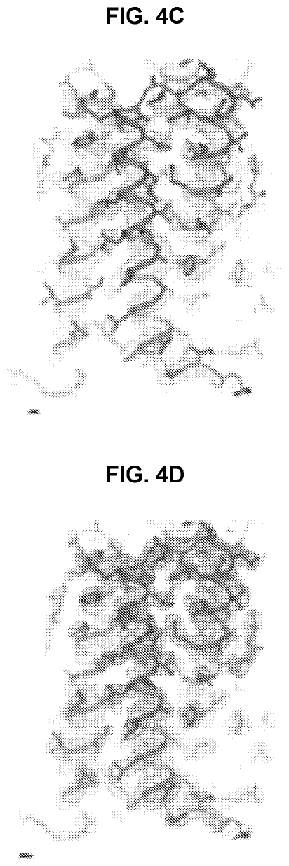

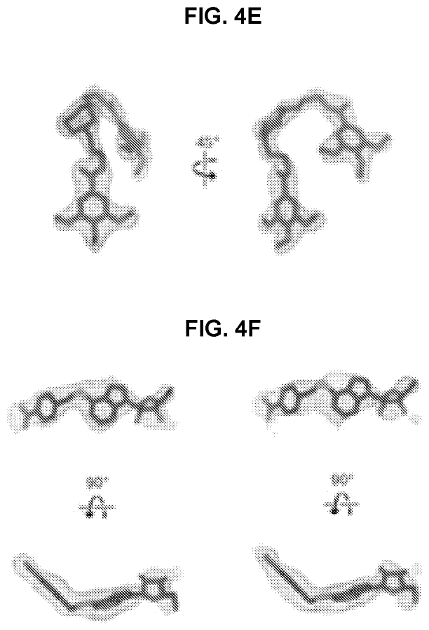

A scintillation proximity assay was developed to measure binding of ³H-NBMPR to hENT1-GFP fusion expressed and detergent solubilized from transiently transfected HEK293T cells (see Methods for details). By successively heating the assay mixture at increasing temperatures and measuring binding signal with scintillation counting, Tₘ values were determined for engineered hENT1 constructs bearing point mutations or truncations of disordered loops. Point mutations were designed based on a consensus approach, which is described in the Methods section of this chapter. A total of 40 point mutations or disordered-loop truncations were screened with this ³H-NBMPR SPA approach (FIG. 4A). Mutations L168F, P175A and N288K were identified to substantially increase Tₘ. Additionally, a disordered loop truncation (A243-274) previously reported to have no effect on general features of hENT1 functional activity was found to enhance stability of the transporter (FIG. 4A). The construct bearing these modifications (referred to as hENT1_{cryst}) retains NBMPR binding as assessed by the ³H-NBMPR SPA-thermostability assay and exhibits a ˜20° C. increase in Tₘ compared to the wild type hENT1 (FIG. 4B). Importantly, hENT1_{cryst} mediates nucleoside transport to a level consistent with a previous report in proteoliposomes (FIG. 3A) and is also functionally active in when expressed in oocytes (FIG. 3B). Additional evidence supporting the stability and functional competency of hENT1_{cryst} is the fact detergent purified hENT1_{cryst} exhibits high levels of binding to ³H-NBMPR, compared to detergent purified wild type (FIG. 3C).

Crystallization and Structure Determination Informed from earlier stability experiments (FIG. 3B), the AdoRI dilazep was included during protein expression and purification of hENT1_{cryst} to further enhance protein stability and maximize the chances of successful crystallization (FIG. 4A). Indeed, dilazep-bound hENT1_{cryst} readily formed plate-like crystals in lipidic cubic phase, which diffracted X-rays beyond 2.3 Å resolution (FIG. 4B, Table 1). Using platinum-soaked crystals, experimental phases to 3.5 Å were obtained by single-isomorphous replacement anomalous scattering (SIRAS) (FIG. 4C, Table 1). Rounds of model building and single-wavelength anomalous dispersion were performed with molecular replacement (MR-SAD) using coordinates initially built into the 3.5 Å experimental maps as a reference model, followed by refinements of the model against the high-resolution native data. The final refined structure is of excellent quality with a Free-R factor of 24% (FIG. 4D, Table 1), and features strong omit density corresponding to dilazep (FIG. 4E). hENT1_{cryst} was also crystallized in the presence of NBMPR, which exhibited merohedral twinning. Phases were obtained from the high-resolution dilazep-bound hENT1 structure by molecular replacement, and refinement was carried out against the X-ray data to 2.9 Å resolution (see Methods). Unmodelled Fₒ-F_{c} density corresponding to NBMPR is strong and enabled accurate modeling, and the final structure features good overall protein geometry (FIG. 4F, Table 1).

TABLE 1
X-ray data collection, phasing, and refinement statistics (dilazep, NBMPR)

| | | |
|---|---|---|
| Dilazep, Native^{b,d} | Dilazep, Pt | NBMPR, Native^{b,d} |
| (PDB 6OB7) | Derivative^{c} | (PDB 6OB6) |

Data Collection

| | | | |
|---|---|---|---|
| Space group | P 3₂ 21 | P 3₂ 21 | P 6₁ |
| Cell Dimensions | | | |
| a, b, c (Å) | 72.0 72.0 173.4 | 72.2 72.2 172.3 | 72.5 72.5 335.7 |
| α, β, γ (°) | 90 90 120 | 90 90 120 | 90 90 120 |
| Resolution (Å) | 62.39-2.30 | 62.53-2.90 | 62.82-2.90 |
| | (2.38-2.30)^{a} | (3.08-2.90) | (3.08-2.90) |
| Rpim | 0.11 (0.56) | 0.07 (0.40) | 0.34 (1.98) |
| l\|σ (I) | 6.0 (1.4) | 8.7 (2.2) | 4.0 (0.8) |
| CC_{1/2} | 0.99 (0.39) | 91.8 (82.4) | 0.99 (0.77) |
| Completeness (%) | 100.0 (100.0) | 0.93 (0.33) | 74.9 (19.3) |
| Redundancy | 4.6 (4.1) | 56.5 (49.8) | 7.8 (8.0) |

Refinement

| | | |
|---|---|---|
| Resolution (Å) | 62.39-2.30 | 62.82-2.90 |
| | (2.38-2.30) | (3.08-2.90) |
| No. reflections | 21962 (1924) | 16459 (442) |
| R_{work}/R_{free} | 0.20/0.24 | 0.21/0.25^{c} |
| No. atoms | | |
| Protein | 5953 | 10,934 |
| Ligand | 602 | 92 |
| Water | 63 | 0 |
| B factors | | |
| Protein | 24.0 | 29.2 |
| Ligand/ion | 39.7 | 30.5 |
| Water | 24.6 | n/a |
| RMS deviations | | |
| Bond lengths (Å) | 0.005 | 0.004 |
| Bond angles (°) | 1.0 | 0.9 |

^{a}Values in parentheses are for highest-resolution shell.
^{b}X-ray data from a single crystal.
^{c}X-ray data from 3 crystals.
^{d}X-ray data anisotropically corrected with the Staraniso webserver.
^{e}Phenix reported R-factors from twin-corrected structure factors (twin operator h, -h-k, -I).

### The ENT Family Protein Fold

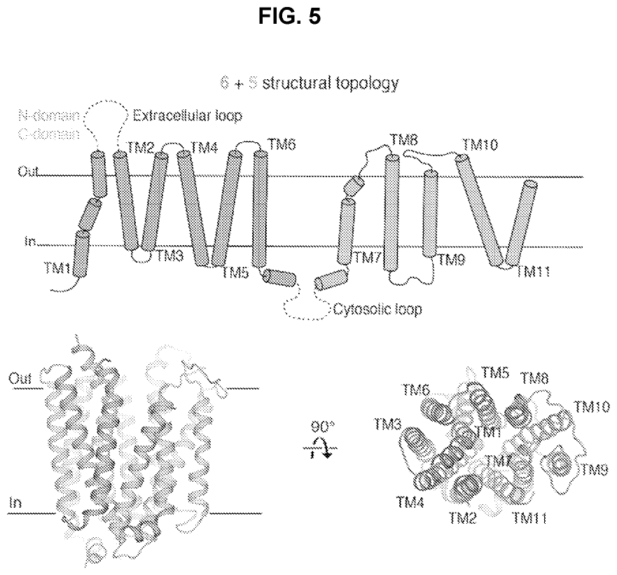

hENT1_{cryst} is a monomer and composed of 11-transmembrane (TM) helices with the N-terminus in the cytosolic side and the C-terminus in the extracellular side (FIG. 5), which is consistent with previous accessibility studies. The first 6 TM form one structural unit, which is termed the N-domain, and the final 5 TMs forms another bundle in which is termed the C-domain (FIG. 5). These bundles exhibit pseudo-symmetry, which is referred to as a “6+5 topology”. This is similar to that of MFS transporters, which exhibit a “6+6 topology”—therefore, this data supports the early postulations that the ENT family protein fold is related to that of MFS.

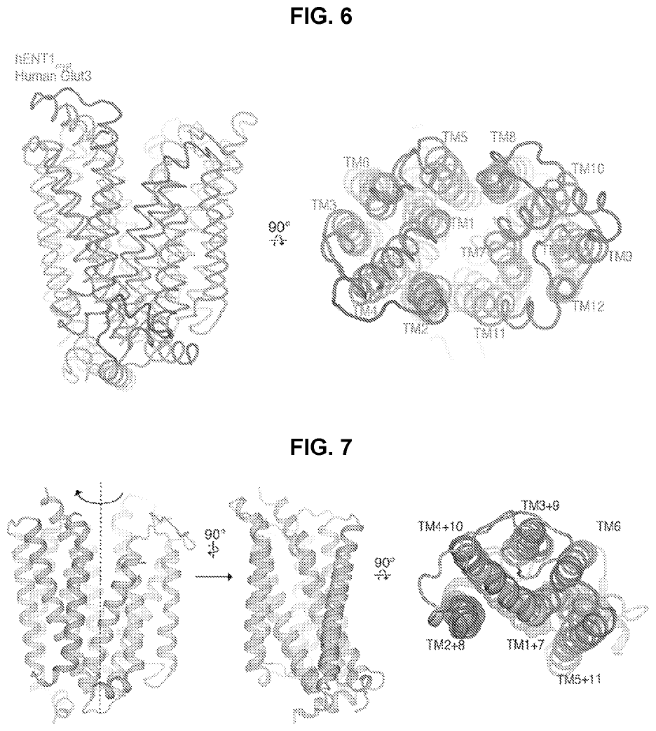

There are interesting differences between the canonical MFS fold and the ENT fold. The hENT1_{cryst} structure was superposed on human Glut3, a representative outward-facing MFS X-ray structure, and despite their low sequence identity (˜17% sequence identity) and global structural similarity (Ca RMSD of 5.0 Å), the first 11 TM of hENT1 follows the overall position of the first 11 TMs (TM1-TM11) (FIG. 6). Structural differences between MFS and ENT folds include the displacement of TM9 in hENT1 relative to MFS, as it appears to be arranged to fit in to the space that is between TM9 and TM12 in MFS (FIG. 6).

Additionally, due to the “6+5 topology” exhibited by hENT1, the structural symmetry between the N and C domains of hENT1 is relatively lower with a Ca RMSD of 4.0 Å, compared to that of ˜3 Å in canonical MFS transporters. The position of TM3 relative to its pseudo-symmetry mate TM9 appears to be most different between the N- and C-domains (FIG. 7).

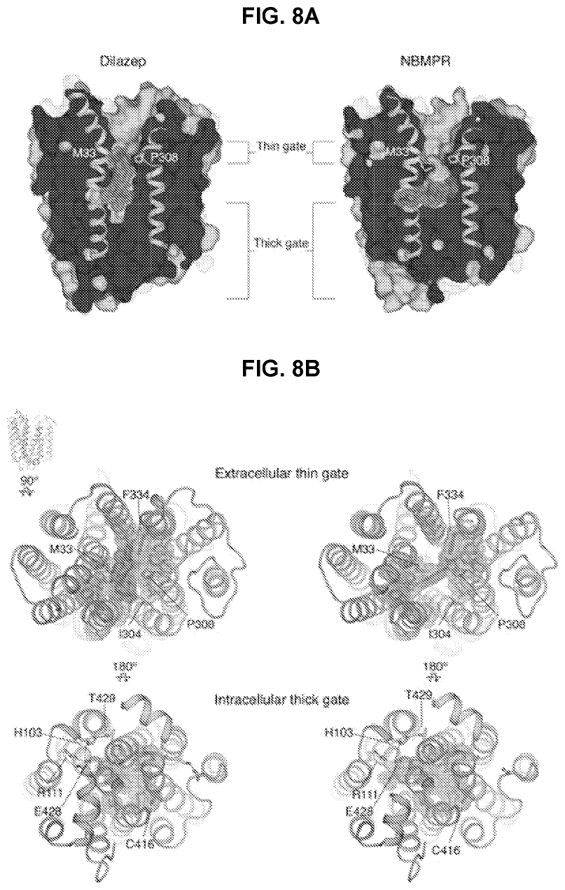

The central cavity of the transporter is accessible to the extracellular side of the membrane and is occupied by ligand in either dilazep-bound or NBMPR-bound structures, suggesting that both structures represent outward-facing conformations. This is consistent with the predictions from previous functional studies, which proposed many AdoRIs recognize an exofacial site on hENT1. The narrowest restriction point at the extracellular side occurs between M33 of TM1 and P308 of TM7. To be consistent with the nomenclatures of other MFS and SLC transporters, this region was termed as the “extracellular thin gate.” One notable difference between the NBMPR and dilazep structures at this particular region—dilazep appears to be preventing complete occlusion of the thin gate, whereas NBMPR sits lower in the transporter cavity and does not physically contact this thin gate (FIG. 8A-B). At the opposite side of the transporter, TM4, TM5, TM10 and TM11 are the four helices featuring the most extensive hydrophobic contacts, fully sealing solvent access from the intracellular side. This “intracellular gate” is fully formed; therefore, it was referred to it as the “intracellular thick gate.”

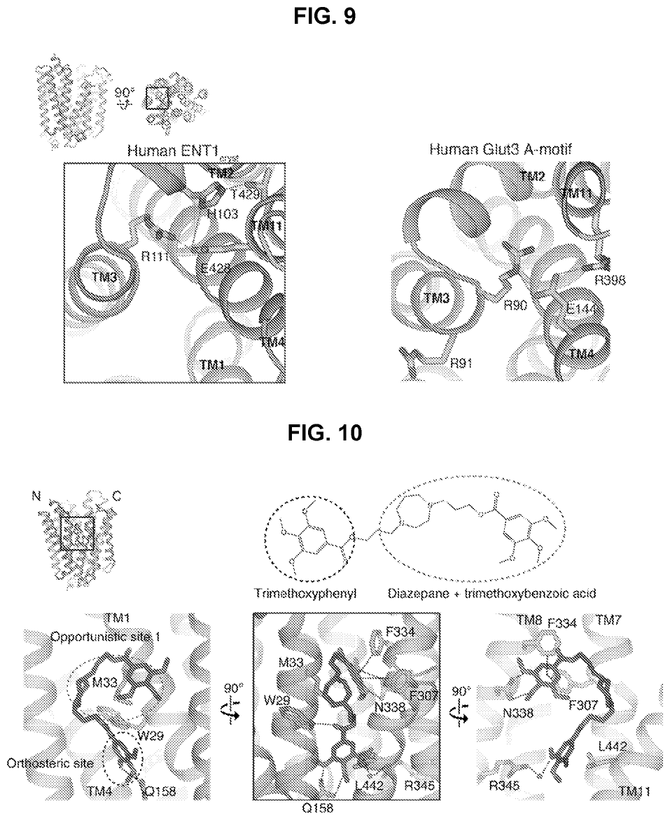

Arg111 and Glu428, residues that are exclusively conserved across mammalian ENTs and highly conserved across the entire ENT family, appear to be further stabilizing the tight contacts between the N- and C-domains at this structural region. It is tempting to speculate an important functional role for these residues based on this observation. This interaction network is similar to the highly-conserved A-motif of MFS, which is located at a structurally analogous position in human Glut3 but bears substantial differences (FIG. 9). This begs the questions whether ENTs exhibit mechanistic differences in the transport cycle compared to MFS transporters.

### Adenosine Reuptake Inhibitor Binding Modes

### Dilazep

Dilazep adopts a crescent conformation within the central cavity of hENT1. Its two trimethoxyphenyl rings occupy different sites: one site is deep within the central cavity, and the other is more proximal to the extracellular side (FIG. 10). Amino acid residues previously implicated in nucleoside recognition are located deep within in the central cavity at an analogous position to one of the trimethoxyphenyl rings. This site represents the nucleoside binding site, and therefore term this region the “orthosteric site.” The remain portion of dilazep protrudes towards the extracellular thin gate and occupies space outside of the central cavity—this particular region is referred to as the “opportunistic site 1”. Important interactions with transporter occur between the orthosteric site trimethoxybenzoic acid group of dilazep and W29 from TM1, along with and Q158 from TM4, both of which are in the N-domain. The central diazepane ring of dilazep directly contacts M33 from TM1 (FIG. 10). This corroborates previous reports which proposed M33 is a significant determinant of the subtype specificity exhibited by dilazep and dipyridamole. The other trimethoxyphenyl ring, which occupies the opportunistic site 1, interacts with numerous C-domain residues—N338 from TM8, and also p-p stacking interactions with F307 (TM7) and F334 (TM8). Of these observed interactions in the structure, W29, F334 and N338 have been previously shown to affect hENT1 inhibition by dilazep when mutated.

### NBMPR

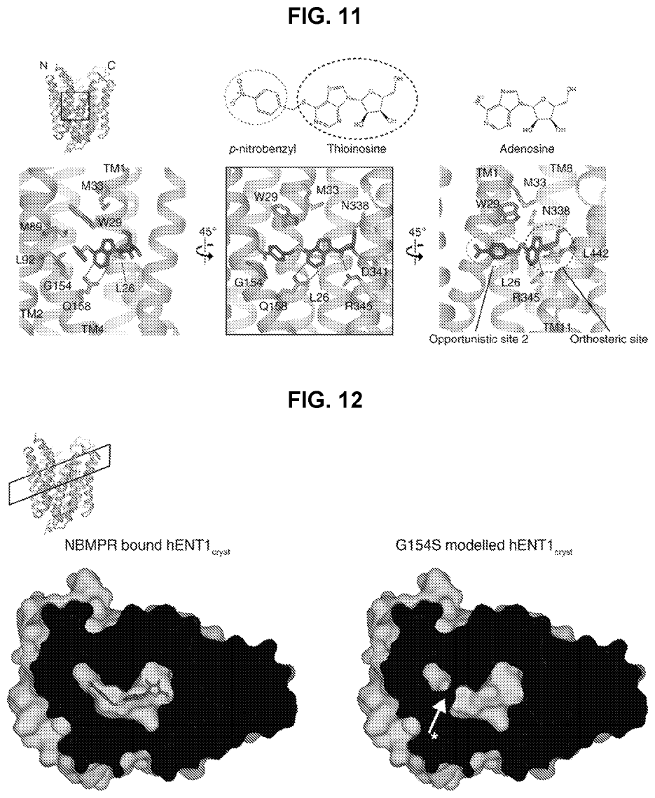

Harboring a thioinosine nucleoside moiety, NBMPR binds lower than dilazep, mostly within the transporter central cavity. Specifically, the nucleoside moiety of NBMPR sits well within the orthosteric site. The 2′-OH and 3′-OH ribose groups interact with the highly conserved residues R345 and D341, respectively (FIG. 11). These residues appear to be functionally important, as it was previously reported the corresponding positions in LdNT2, an ENT family member from L. donovani, are critical for nucleoside transport activity. Q158, another highly conserved residue in the orthosteric site, coordinates to the N-1 and N-3 amino groups purine ring, indicating it may be an important feature of nucleoside recognition by hENT1. Residues L26, M89, L92 and L442 also surround the purine moiety of NBMPR—notably, L26 and L442 flank the purine ring (FIG. 11), supporting a previous finding where L442I converts nucleoside preference to uridine over adenosine in hENT1. Additionally, M89 and L92 were also implicated in both purine and NBMPR binding.

Adenosine recognition by hENT1 is likely similar to the thioinosine binding by hENT1 in the structure. In total, hENT1 employs two highly conserved charged residues for ribose coordination. Nucleobase recognition is facilitated by polar coordination through 0158 in addition to hydrophobic contacts within the central cavity.

The central cavity of hENT1 features a deep-hydrophobic pocket protruding into the transporter N-domain, which is lined by TM1, TM3 and TM4. In the NBMPR-hENT1 complex structure, this cavity is proximal to the purine ring of NBMPR, and the p-nitrobenzyl group protrudes into this hydrophobic cavity (FIG. 11). Considering this site is distinct from the “orthosteric site” and the “opportunistic site 1” in which dilazep occupy, this region was termed “opportunistic site 2”. Glycine-154 is in close proximity to the p-nitrobenzyl ring at this site—intriguingly, the amino acid residue at the equivalent position in hENT2 or hENT3 is serine, or cysteine in hENT4. Previous work has shown substitution to serine at this position in a wild type hENT1 background was shown to result in a drastic reduction in NBMPR inhibitory potency (˜2,500-fold). Therefore, it G154 was previously proposed to be an important contributor to the high level of subtype specificity displayed by NBMPR. Such a substitution in the NBMPR-hENT1 structure narrows the hydrophobic cavity at this position, which would sterically hinder p-nitrobenzyl ring occupancy (FIG. 12). This provides a plausible explanation for the mutational effect of G154S, and the molecular basis for the high-degree of subtype specificity exhibited by NBMPR.

### Shared and Distinct Sites

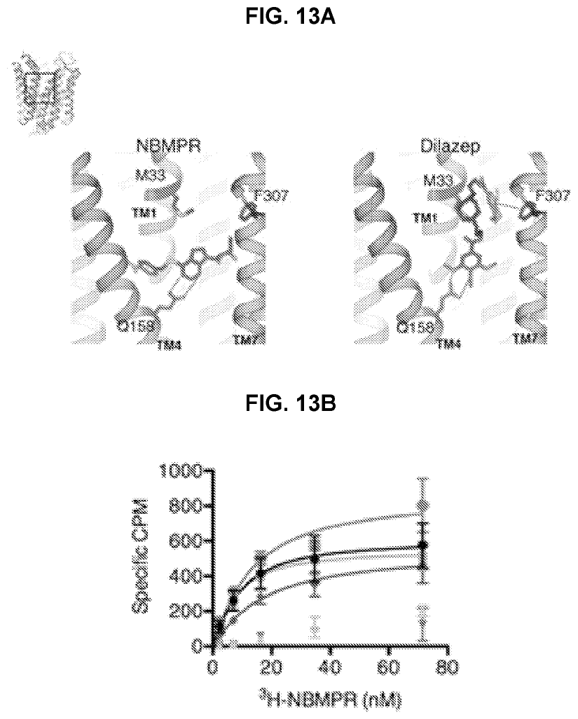

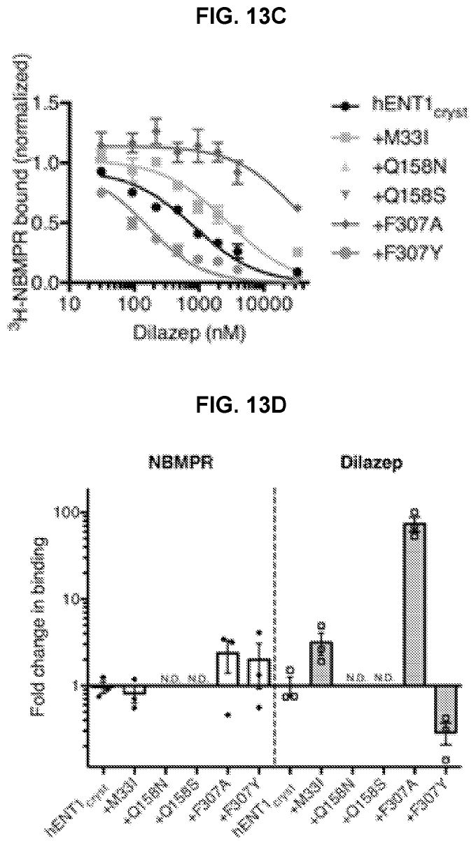

While the inhibitors dilazep and NBMPR both occupy the orthosteric site, they form additional transporter interactions at their respective opportunistic sites. Glutamine-158 forms important interactions with either dilazep or NBMPR in the orthosteric site (FIG. 13A). This is substantiated by the equilibrium binding data, as both Q158S and Q158N ablate 3H-NBMPR binding capacity (FIG. 13B). Since transporter elements within opportunistic site 1 appear to be utilized for dilazep binding only, but not for NBMPR binding, F307 was mutated since it has not been previously studied. Using the equilibrium binding assay, NBMPR binding was tested via direct ³H-NBMPR SPA, while dilazep binding was measured by displacement of bound ³H-NBMPR. As an assay control, M33I was assessed, an element of opportunistic site 1, and observed ˜3-fold increase in apparent K_{d} for dilazep, while there is no noticeable change in Kd for NBMPR (FIG. 13B-D). This finding is consistent with previous reports on inhibitor sensitivities to substitutions at position 33 of hENT1. Position 307 was mutated and a ˜90-fold increase in apparent K_{d} was detected for dilazep in F307A, whereas the conservative mutation F307Y resulted in a ˜4-fold decrease in apparent K_{d} for dilazep. Neither F307A nor F307Y had a noticeable effect on NBMPR binding in hENT1 (FIG. 13B-D). This finding is in line with the structural observations that the opportunistic site 1 contributes substantially to the binding energetics of dilazep and not NBMPR. Further, mutation of Q158 at the shared orthosteric site considerably reduces NBMPR binding (owning to fact the equilibrium binding assay relies on NBMPR binding as a readout, dilazep binding via cold-competition was not determined for Q158N or Q158S).

### Proposed Structural Mechanisms of Action by Two Different AdoRIs

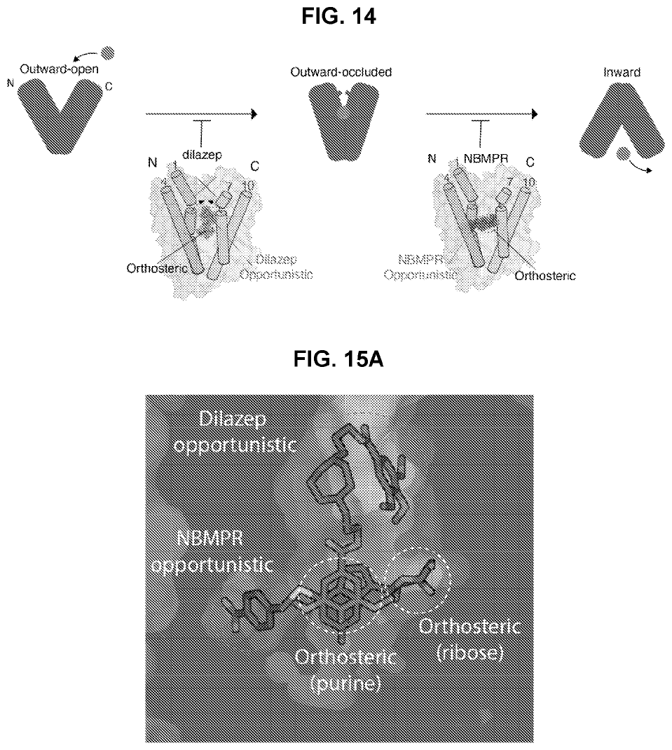

The structural data presented here reveals the AdoRIs dilazep and NBMPR occupy two distinct opportunistic sites, indicating they may exert their inhibitory action on hENT1 in different manners. Considering hENT1 exhibits a two-domain architecture hENT1 and structural similarities with MFS (FIG. 6), the transporter will likely utilize global rocker-switch-like movements for its alternating access mechanism of adenosine and nucleoside-analog drug transport. In the context of such a transport mechanism, the two inhibitor-bound states lock hENT1 in outward-facing conformations. Preceding the transporter transition to the inward-facing state, the extracellular thin gate would have to completely close, forming an extracellular thick gate, followed by N- and C-domain reorientation (FIG. 14). While both NBMPR and dilazep occupy the orthosteric site, opportunistic site 1 of dilazep is located at the region including the extracellular thin gate. Therefore, dilazep sterically blocks complete extracellular gate closure, which is a step required for the transition into the inward-facing state (FIG. 14). Prohibiting gate formation has been observed in numerous inhibitors of MFS and sodium-coupled neurotransmitter transporters.

Extracellular thick gate formation is not sterically hindered in the case of NBMPR. Opportunistic site 2 is occupied by the p-nitrobenzyl group of the inhibitor, while the adenosine-like thioinosine group occupies the orthosteric site of the transporter. Previous work has highlighted the large conformational changes substrate-lining TMs undergo during the transport cycle. Therefore, TM1, TM3, and TM4 surrounding the opportunistic site 2 may be important for such a conformational transition in hENT1. Thus, occupancy of this site would hinder key conformational rearrangements of the N-domain required during the functional cycle of hENT1 (FIG. 14). Currently, all reported inhibitor-bound transporter structures feature some sort of steric hindrance of gate closure. The inhibitory mechanism proposed for NBMPR is unique.

Prior to this work, the molecular architecture of the SLC29 family of nucleoside transporters was unknown and thought to adopt the highly conserved MFS transporter fold (6). The SLC29 family shows interesting differences from MFS. The 11-TM topology of human ENT1 has warranted interesting questions from an evolutionary standpoint. Considering the structures of semi-SWEET and SWEET transporter structures, it is currently thought each lobe (N- and C-domain) of MFS can be further segmented into a 3+3 TM assembly. It is reasonable to conceive the 3 TM bundles in the MFS fold (TMS 1-3, 4-6, 7-9, 10-12) are the minimal structural units with which a functional transporter can be assembled. This indicates the SWEET and semi-SWEET families may represent evolutionary ancestors to MFS, where gene duplication events would lead to early MFS members. Consistent with nomenclature currently used in the field, the 3 TM units consists of a successive arrangement of A-, B- and C-helices. Human ENT1, a functional human transporter, exhibits a protein fold that does not follow the 12 TM topology of MFS or the 3 TM building block strategy from SWEET transporters. This poses the question—what are the minimal structural requirements are to build a functional transporter? A current hypothesis, based on available structural data, is that A- and B-helices contribute most extensively to transporter gating and thus the transport cycle—is it possible the C-helices (TMs 3, 6, 9 or 12) are less critical for transporter function.

From a pharmacological standpoint, the structural and mutagenesis data demonstrates how two chemically unrelated AdoRIs, the non-nucleoside vasodilator dilazep and nucleoside analog NBMPR, potently inhibit hENT1. Further, the data suggests NBMPR inhibits the transporter in a unique manner, while dilazep employs a more common “steric-prevention of gate-closure” mechanism.

### Rational Design of Novel Adenosine Reuptake Inhibitors

Intervening in adenosinergic signaling via reuptake inhibition of extracellular adenosine holds therapeutic promise for the treatment of numerous disorders. It is important to note drug discovery and lead optimization programs have largely failed to produce novel AdoRIs or optimize pharmacological properties of current AdoRIs. This was due to the lack of structural information concerning the target of AdoRIs, which motivated the rational design of novel AdoRIs using the preliminary hENT1-inhibitor structures as a starting point.

### Structure Guided Inhibitor Design

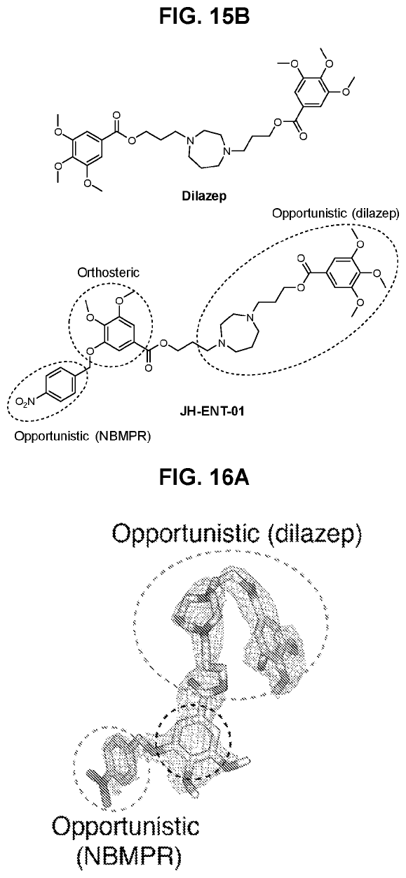

The AdoRIs NBMPR and dilazep occupy distinct opportunistic sites in hENT1 but share the orthosteric site. Importantly, the opportunistic moiety of NBMPR (p-nitrobenzyl ring) directly contacts G154. This position is serine (hENT2 or hENT3) or cysteine (hENT4) in ei (equilibrium-insensitive) ENT subtypes, indicating that G154 is a key feature of the extreme subtype selectivity exhibited by NBMPR. The AdoRI dilazep also exhibits a high level of specificity to hENT1 over other ENT subtypes. Under the premise the distinct opportunistic moieties of dilazep or NBMPR contribute substantially to their potency and subtype specificity, it was thought that combining chemical elements of these two inhibitors would lead to an AdoRI with even higher target potency and subtype specificity, while also improving drug properties (FIG. 15A). The group of Jiyong Hong in Duke Chemistry first synthesized a “NBMPR-DZ hybrid analog,” which is termed JH-ENT-01, by simply grafting the p-nitrobenzyl of NBMPR onto a single trimethoxyphenyl group of the dilazep chemical structure (FIG. 15B).

### Crystallization and Structure Determination

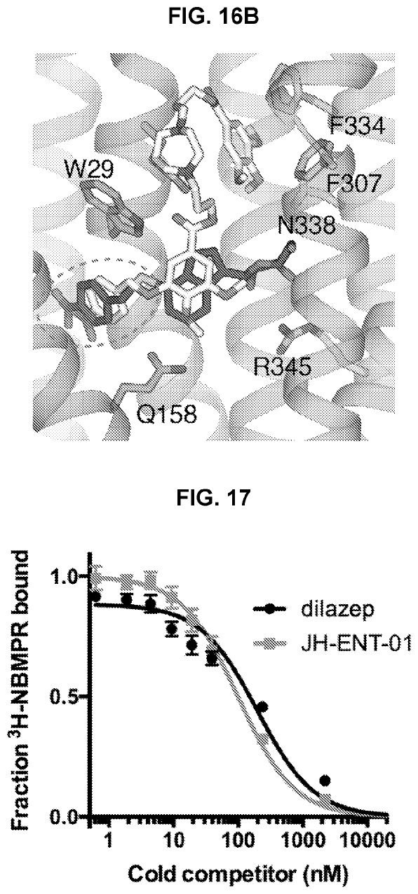

Thermostabilized hENT1_{cryst} was purified in the presence of JH-ENT-01. Crystals were readily obtained in lipidic cubic phase, yielding diffraction of X-rays beyond 3 Å. Using the high-resolution dilazep-bound hENT1_{cryst} structure as a search model, the X-ray structure was solved to 2.7 Å resolution with molecular replacement (Table 2, FIG. 16A-B). A strong unmodelled density corresponding to JH-ENT-01 was present early in X-ray data refinements, enabling model building of the novel inhibitor (FIG. 16A-B).

The overall transporter conformation of JH-ENT-01 bound hENT1_{cryst} is nearly identical to the dilazep-bound structure (RMSD=0.05 Å). The novel inhibitor exhibits a similar binding pose to dilazep when comparing analogous chemical positions between the structures—however, the additional p-nitrobenzyl group occupying the opportunistic site 2 but appears slightly rotated relative to NBMPR upon structural superposition (FIG. 16A-B).

TABLE 2
X-ray data collection and refinement statistics (JH-ENT-01)

JH-ENT-01, Native^{b} (PDB ID XXXX)

Data Collection

| | |
|---|---|
| Space group | P 3₂ 21 |
| Cell dimensions | |
| a, b, c (Å) | 72.41 72.41 173.92 |
| α, β, γ (°) | 90 90 120 |
| Resolution (Å) | 62.71-2.70 (2.83-2.70)^{a} |
| Rpim | 0.16 (1.05) |
| l\|σ (I) | 3.9 (1.1) |
| CC1_{/2} | 0.97 (0.33) |
| Completeness (%) | 99.8 (98.8) |
| Redundancy | |

Refinement

| | |
|---|---|
| Resolution (Å) | 50.64-2.69 (2.79-2.69) |
| No. reflections | 15041 |
| R_{work}/R_{free} | 0.23/0.26 |
| No. atoms | |
| Protein | 3108 |
| Ligand | 313 |
| Water | 16 |
| B factors | |
| Protein | 46.78 |
| Ligand/ion | 61.31 |
| Water | 44.55 |
| RMS Deviations | |
| Bond lengths (Å) | 0.62 |
| Bond angles (°) | 0.003 |

^{a}Values in parentheses are for highest-resolution shell.
^{b}Data merged from 5 crystals.

### Functional Characterization of Novel Inhibitors

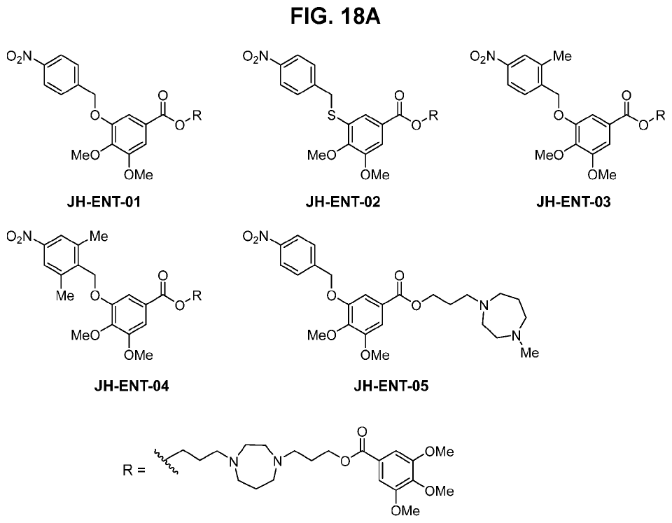

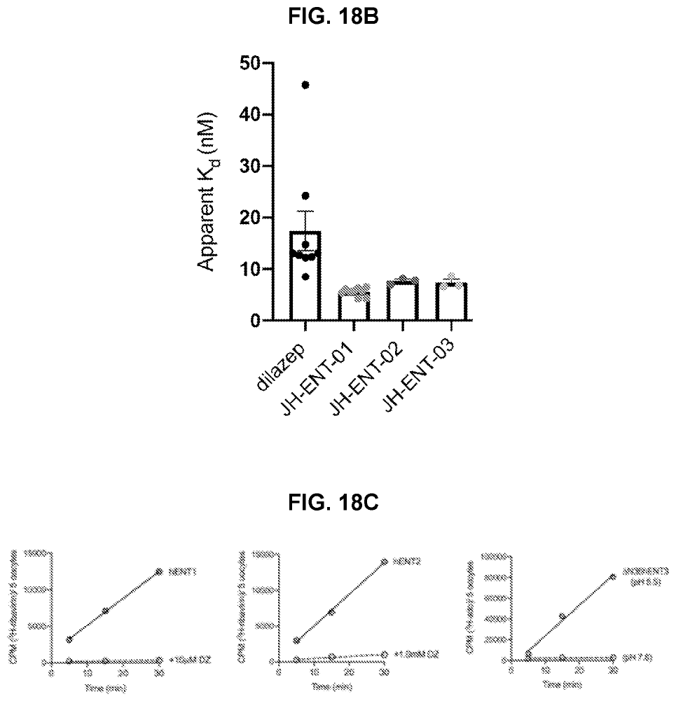

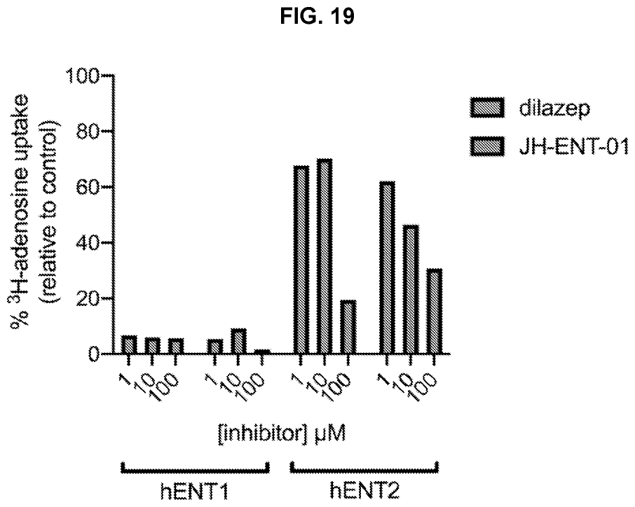

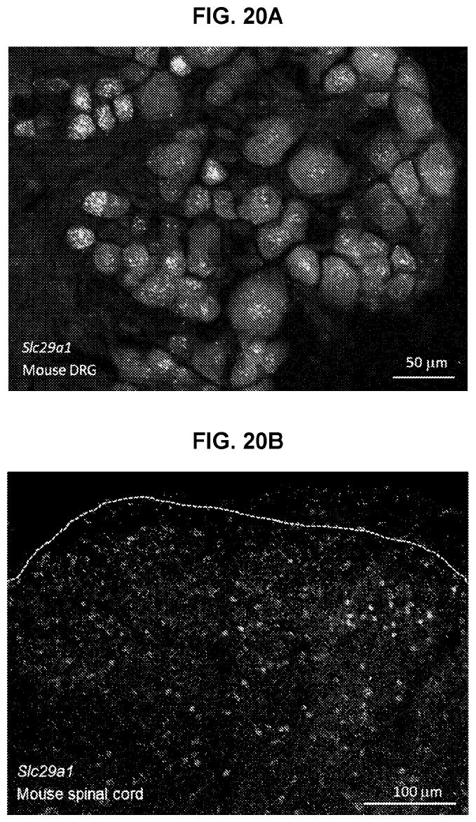

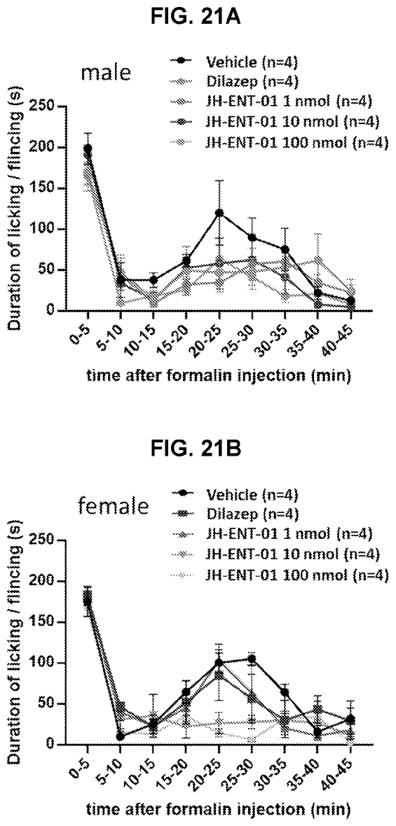

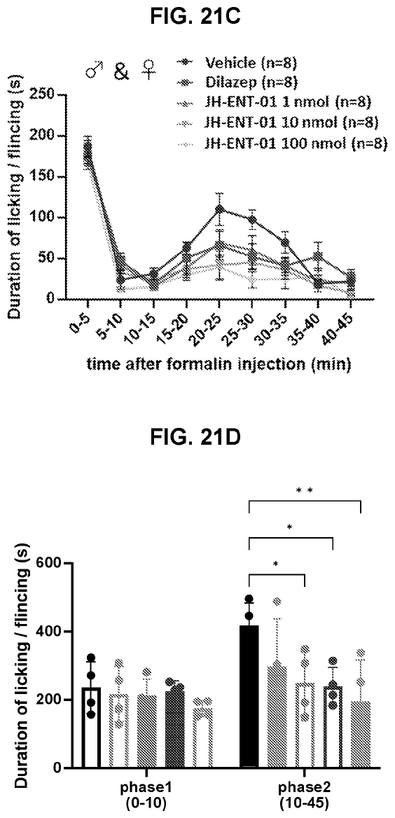

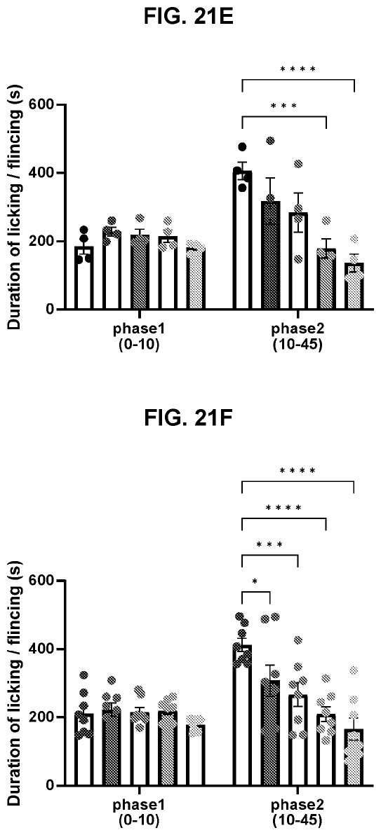

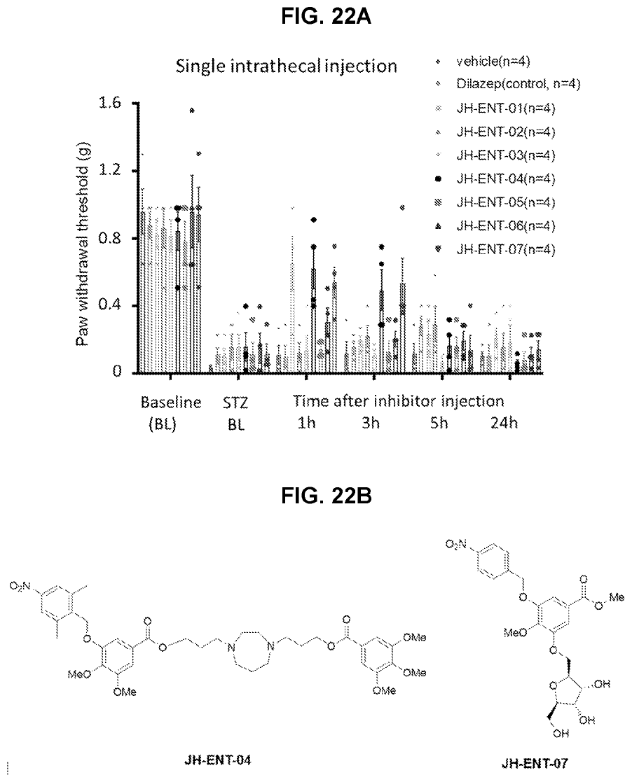

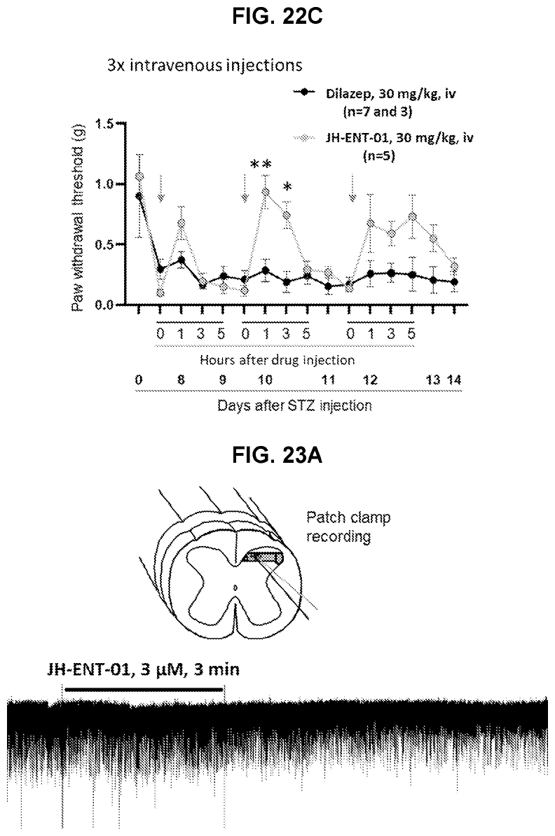

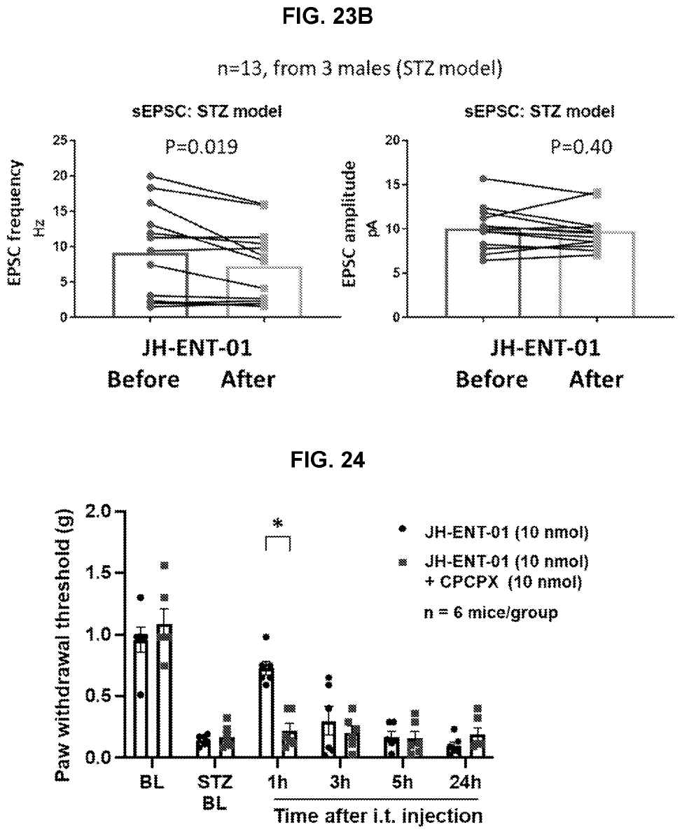

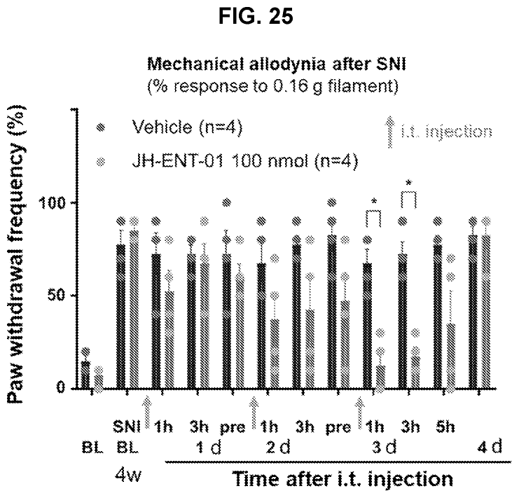

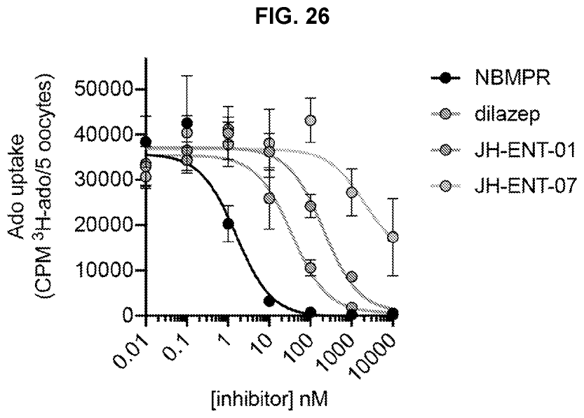

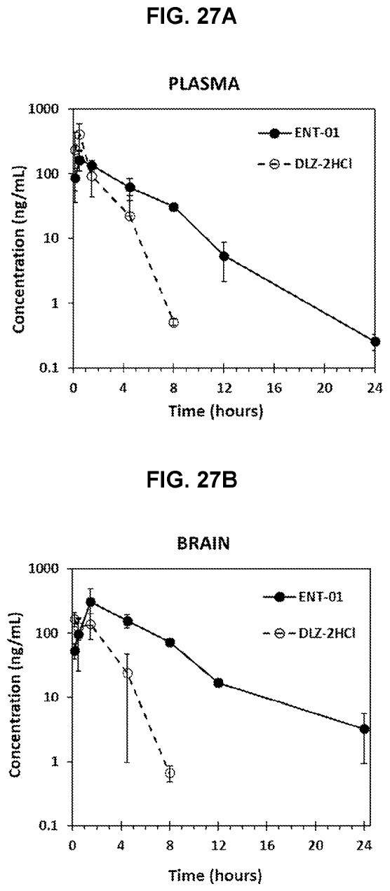

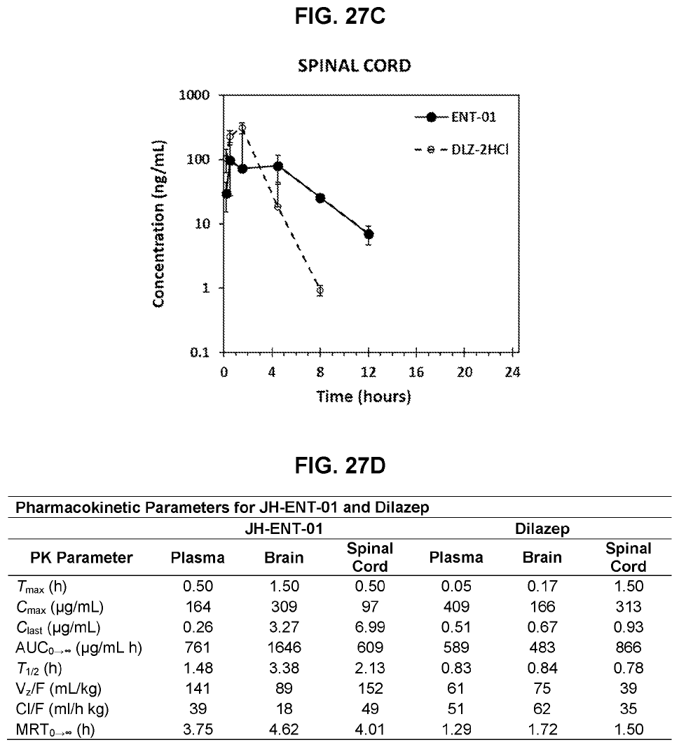

The binding of JH-ENT-01 to detergent purified hENT1_{cryst} was tested with cold-competition SPA and found that the novel inhibitor exhibits a ˜2 fold increase in binding affinity (FIG. 17). Informed by this result, and the structural observation that the additional p-nitrobenzyl ring is slightly rotated in the hENT1 opportunistic site 2, relative to NBMPR, the Hong group synthesized additional novel inhibitors with various modifications off of the NBMPR-DZ hybrid analog scaffold (FIG. 18A). Rationally modified compounds may further improve target potency and subtype specificities, however compounds JH-ENT-02 and JH-ENT-03 exhibit similar affinities to hENT1_{cryst} compared to JH-ENT-01 (FIG. 18B). The subtype specificities for these compounds are currently unknown and pending further investigation with functional assays. Towards this end, established assays in oocytes permitted the measurement of nucleoside transport activity for multiple ENT subtypes (FIG. 18C) and the novel inhibitors were tested for their activities on different ENT subtypes (FIG. 19).

### Pharmacophore for AdoRIs Targeting Human ENT1

The data presented here highlights the feasibility of structure-guided inhibitor design, using two different hENT1 inhibitors as a starting point. The initial structural data and preliminary functional experiments provide a glimpse into the pharmacophore required for AdoRIs targeting hENT1. The minimal requirement for a pharmacologically active AdoRI is occupancy of the orthosteric site and at least one of the two opportunistic sites. While opportunistic site 1 features transporter elements at the extracellular gate, opportunistic site 2 is a small hydrophobic cavity adjacent to the orthosteric site. The JH-ENT-01 bound hENT1_{cryst} structure provides a proof-of-principle that both opportunistic sites can be simultaneously occupied. This results in an AdoRI with improved binding to target compared to dilazep (FIG. 17). Further, preliminary data suggests JH-ENT-01 retains potent inhibition activity against hENT1 mediated uptake (FIG. 19). The hypothesis that the subtype specificity exhibited by an AdoRI would be mediated by interactions within the two opportunistic sites, and that JH-ENT-01 would exhibit higher specificities compared to dilazep or NBMPR alone, remains to be systematically interrogated.

This structure-guided rational approach to novel AdoRI design has yielded multiple new inhibitors. Of the compounds tested, all retain high affinity binding to target. This systematic approach will enable the identification of AdoRIs with improved pharmacological properties, which would have applications to ischemia-reperfusion injury, neurological disorders, and pain management.

### Example 2

### Synthesis of JH-ENT Compounds

### Scheme Overviews

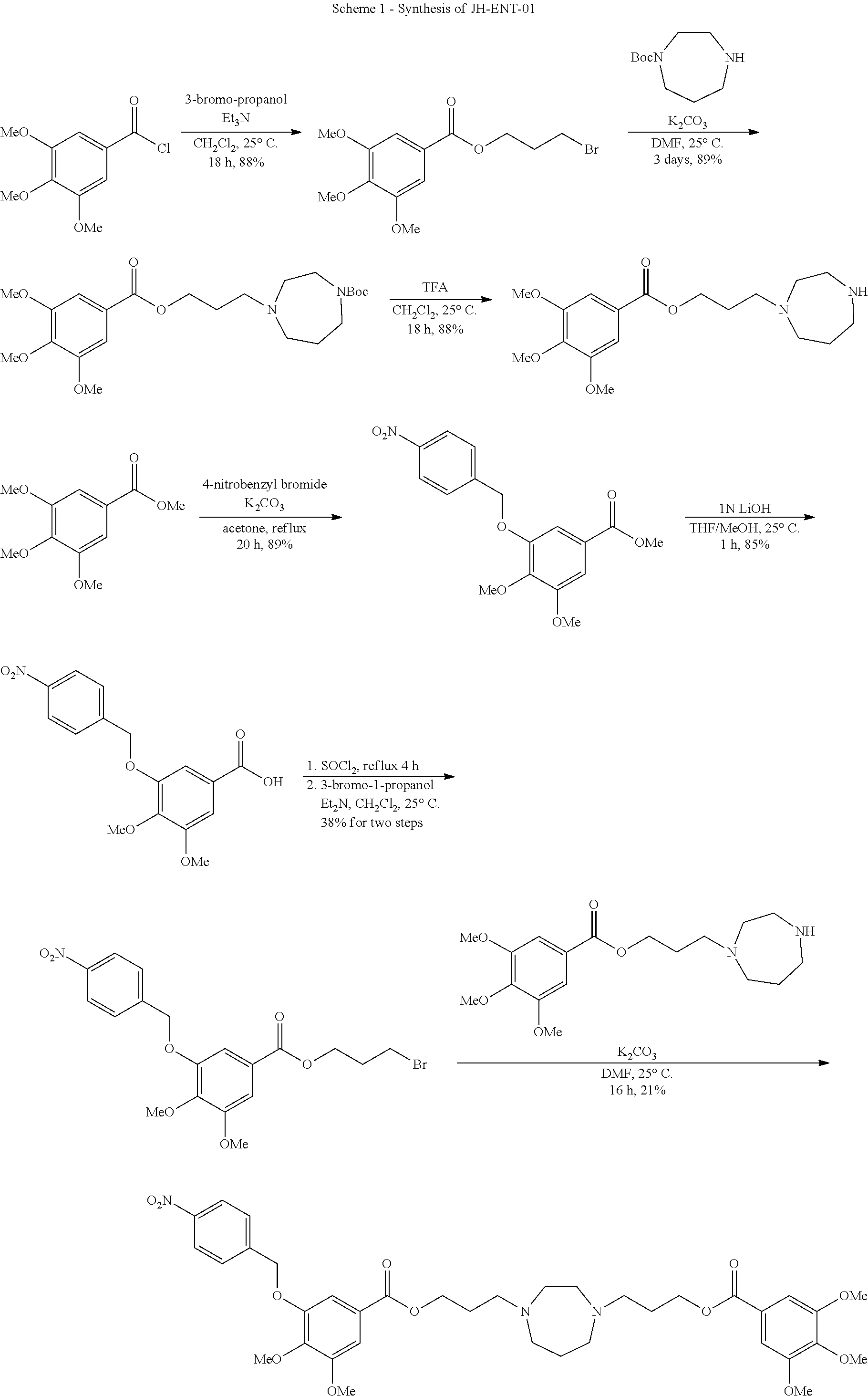

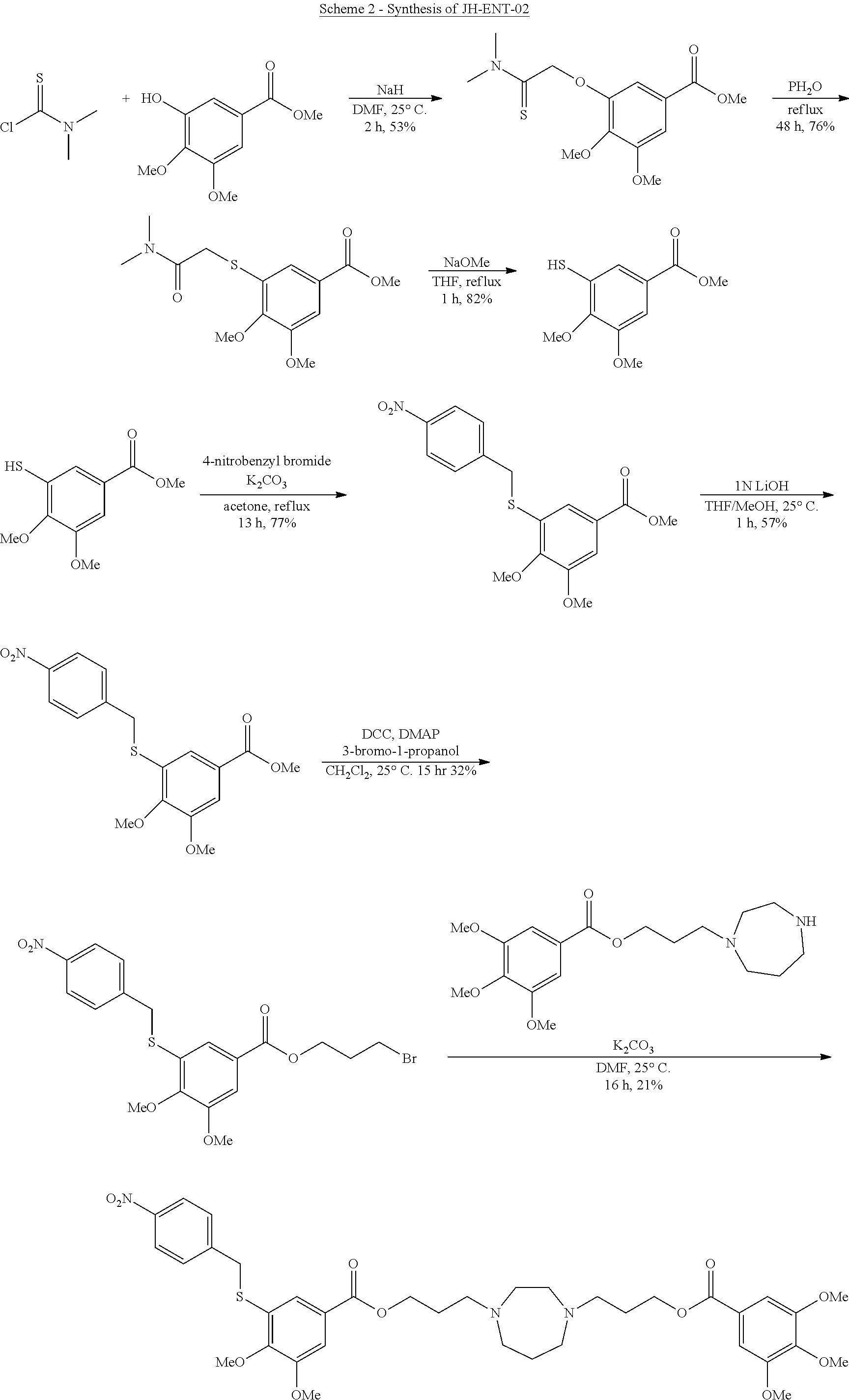

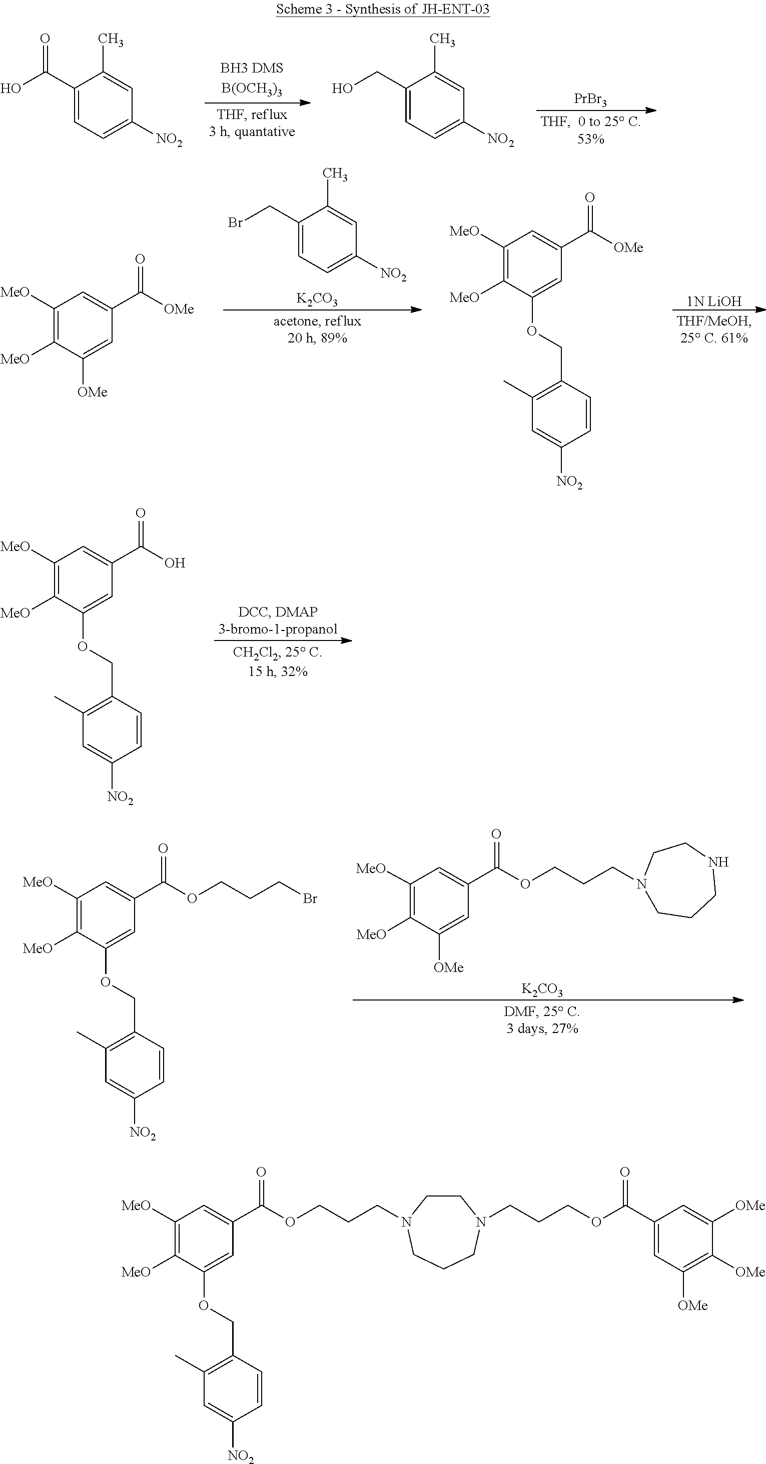

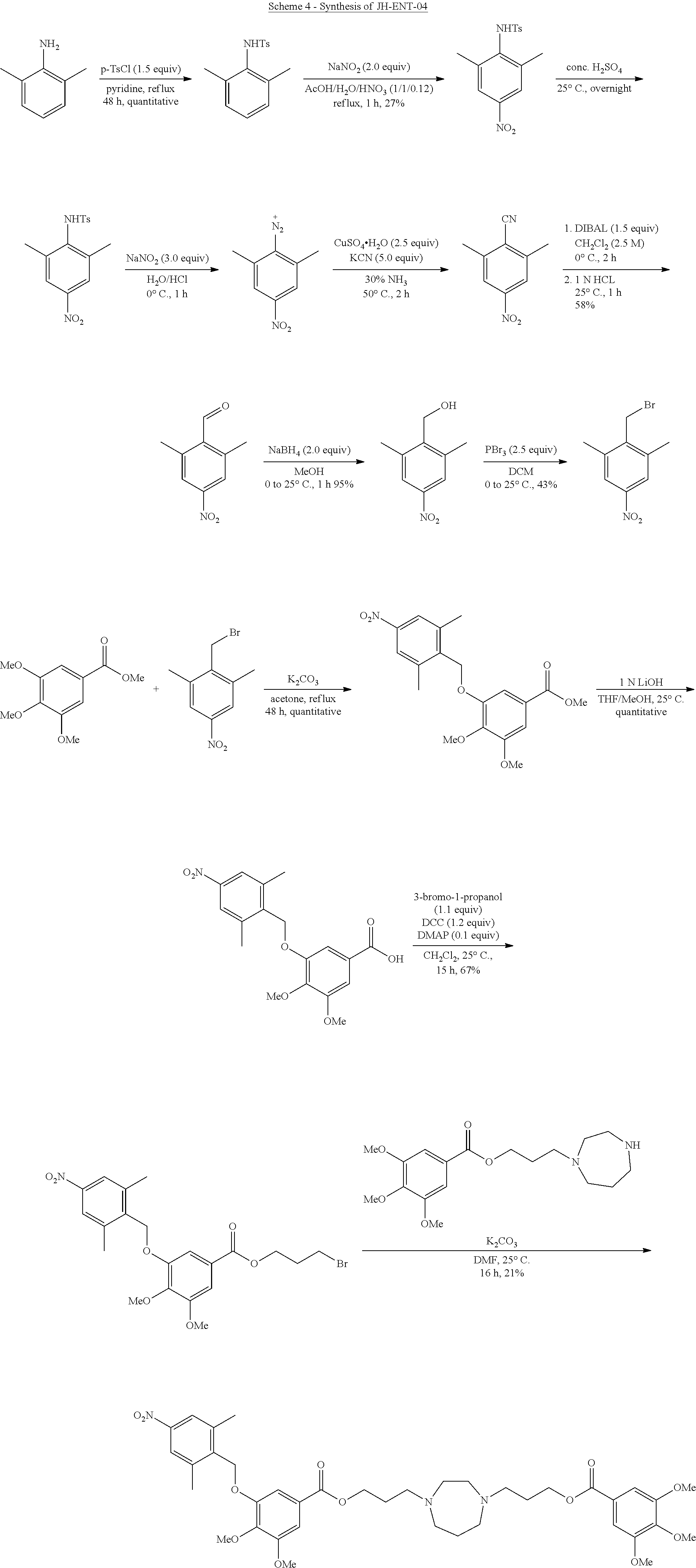

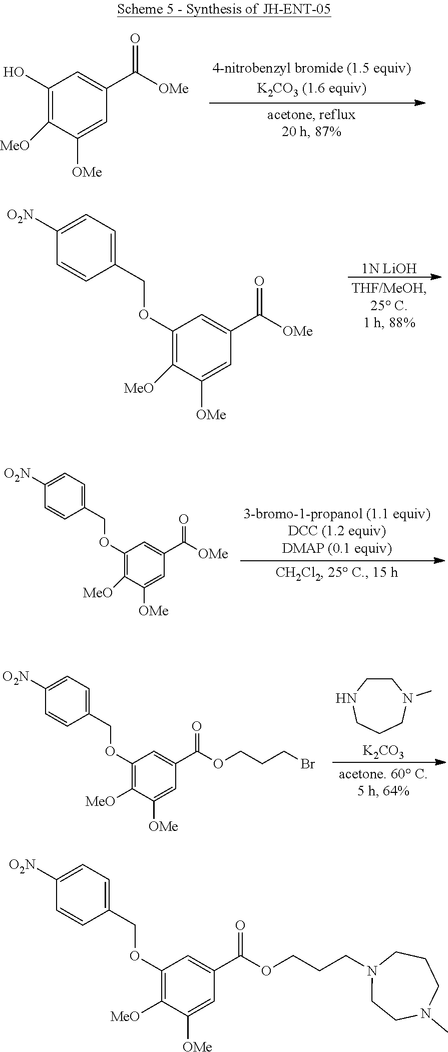

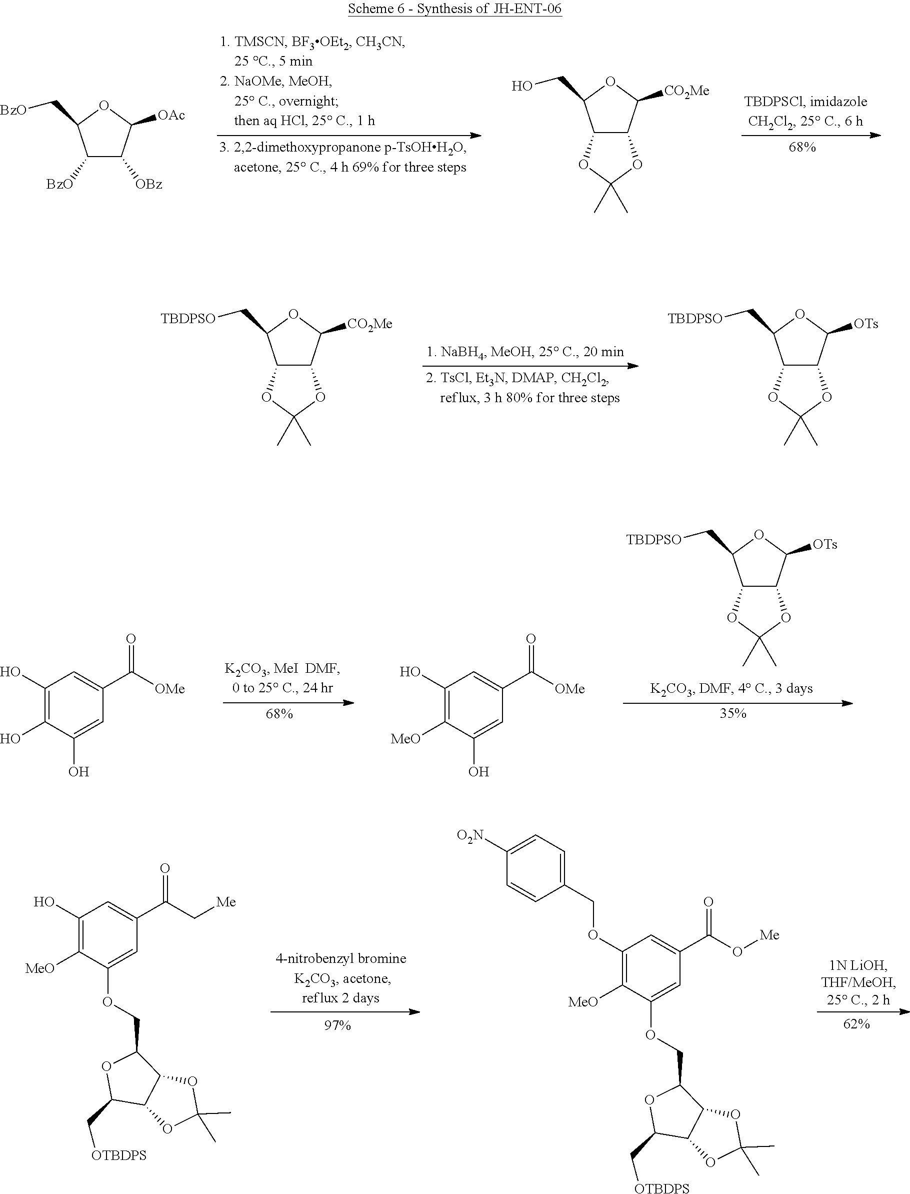

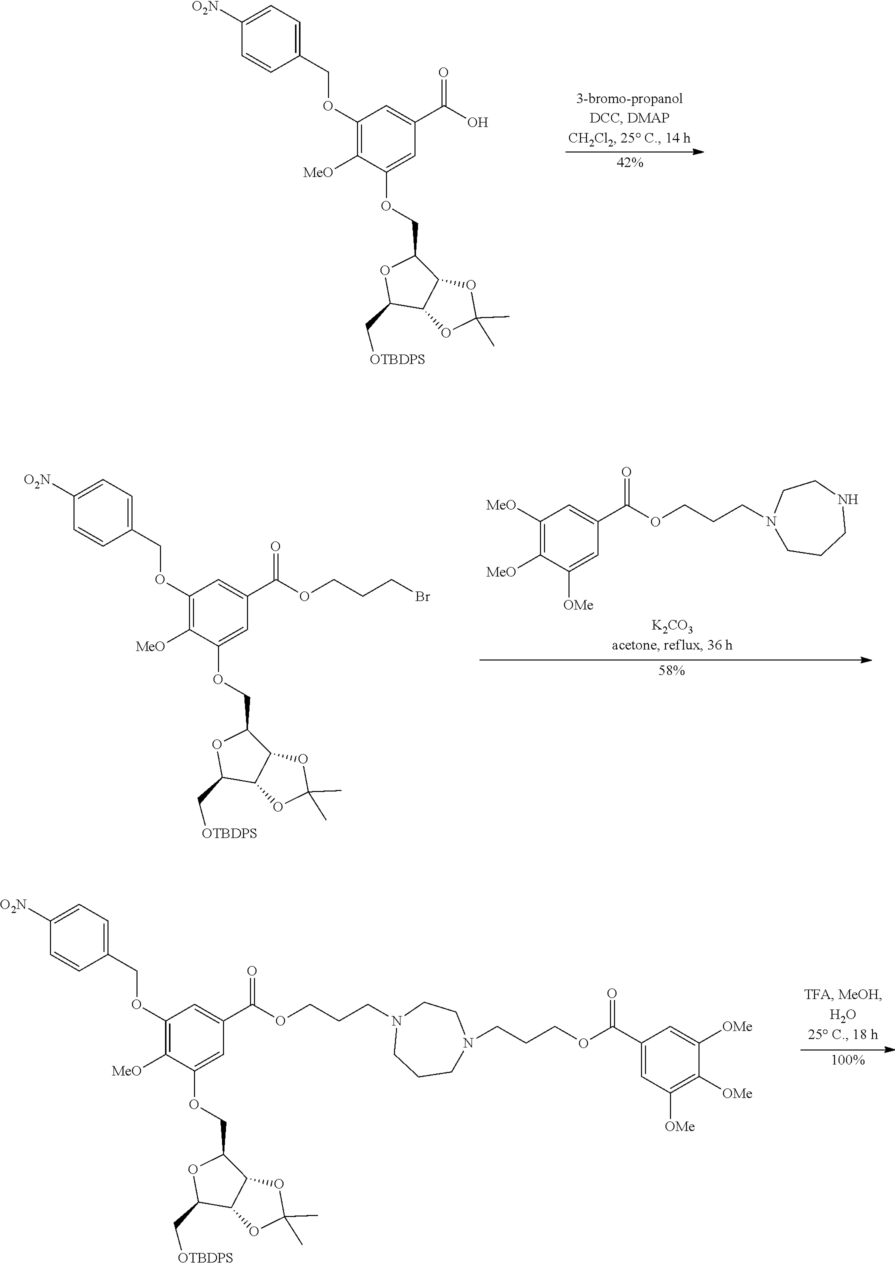

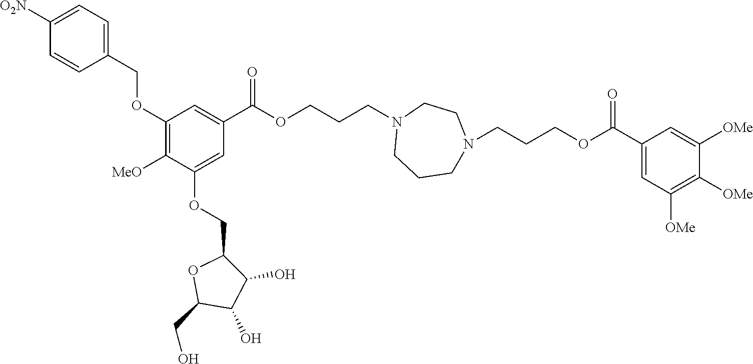 [SMILES: COc1cc(C(=O)OCCCN2CCCN(CCCOC(=O)c3cc(OCc4ccc([N+](=O)[O-])cc4)c(OC)c(OC[C@@H]4O[C@H](CO)[C@@H](O)[C@H]4O)c3)CC2)cc(OC)c1OC]

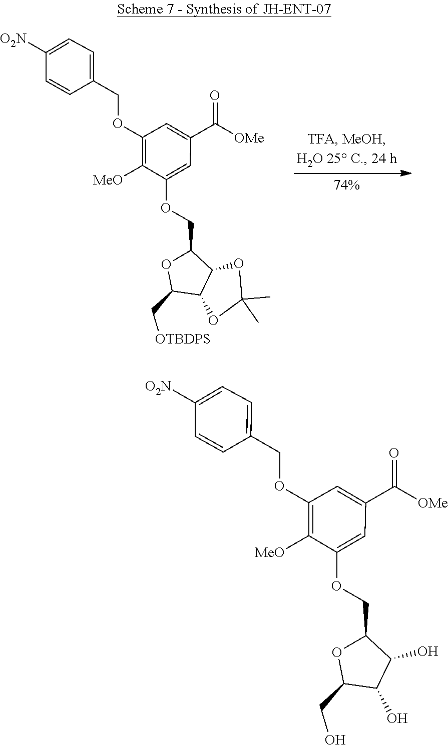

### JH-ENT-01 Synthesis

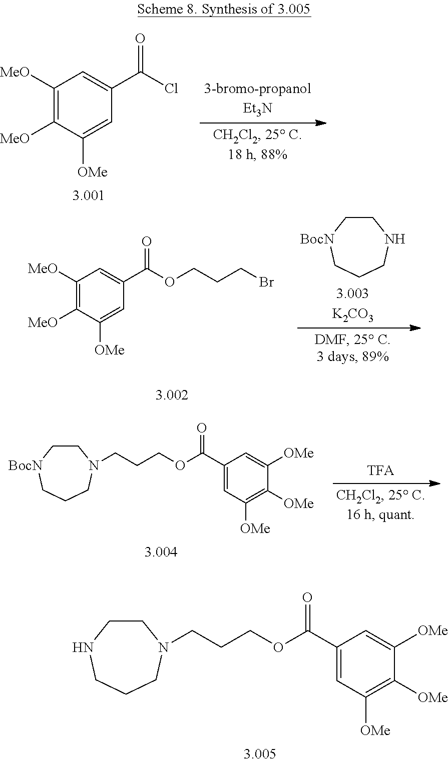 [SMILES: COc1cc(C(=O)Cl)cc(OC)c1OC ; COc1cc(C(=O)OCCCBr)cc(OC)c1OC ; CC(C)(C)OC(=O)N1CCCNCC1 ; COc1cc(C(=O)OCCCN2CCCN(C(=O)OC(C)(C)C)CC2)cc(OC)c1OC ; COc1cc(C(=O)OCCCN2CCCNCC2)cc(OC)c1OC]

The synthesis began with the right-hand portion of the hybrid analogs, homopiperazine 3.005 (Scheme 8). First 3-bromo-1-propanol was coupled to acyl chloride 3.001 to provide bromide 3.002, which was then coupled to 1-Boc-homopiperazine in the presence of K₂CO₃. Finally, TFA deprotection provided homopiperazine 3.005, quantitatively. Next, to build up the left-hand side, phenol 3.006 was coupled to 4-nitrobenzylbromide with the addition of K₂CO₃ under refluxing conditions. The resulting ester (3.007) was hydrolyzed with 1 N LiOH and converted to an acyl chloride intermediate upon treatment with SOCl₂. The acyl chloride was then coupled to 3-bromo-1-propanol under basic conditions to provide bromide 3.009. Finally, bromide 3.009 was coupled to homopiperazine 3.005, in the presence of K₂CO₃ to provide JH-ENT-01.

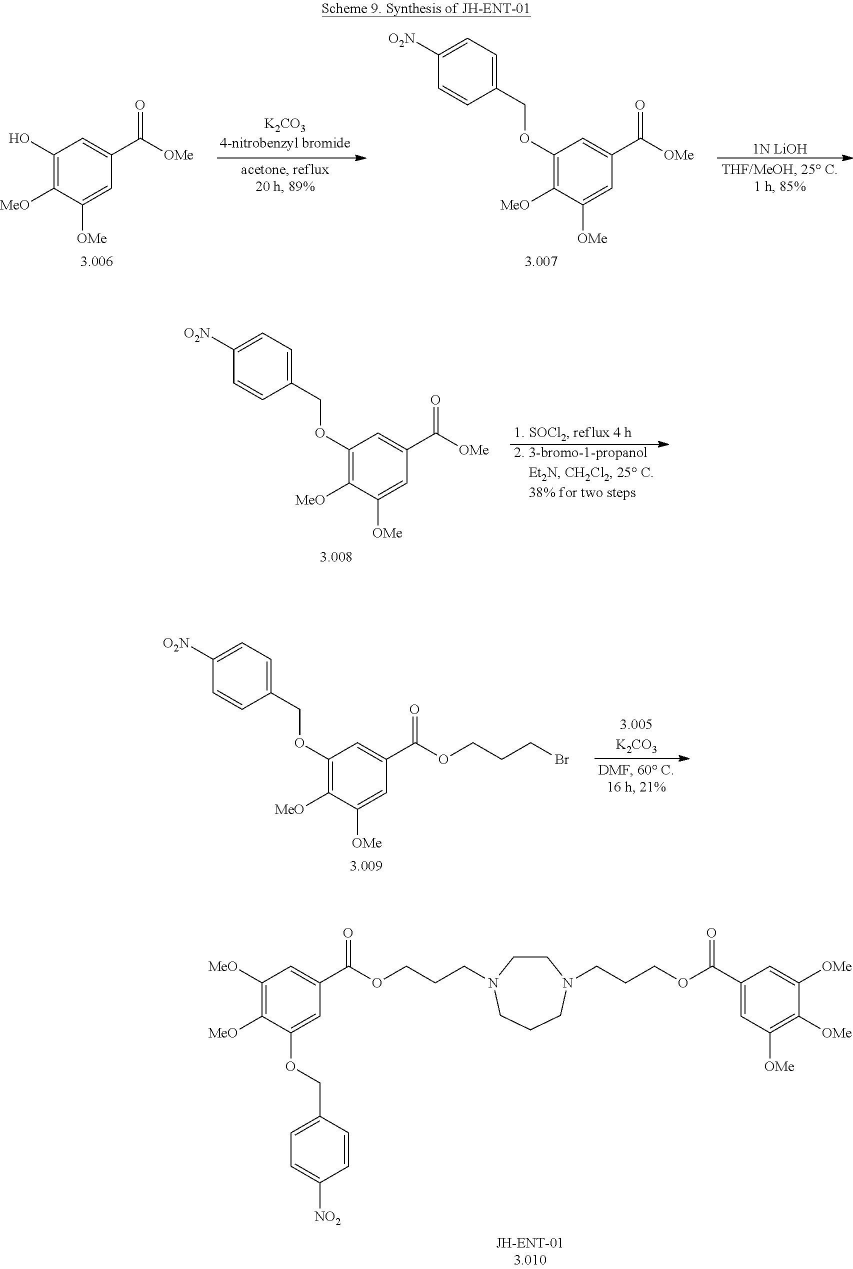

### Design and Synthesis of ENT1 Inhibitor Library

Based on the analysis of JH-ENT-01's binding mode, a small library of inhibitors was designed to develop preliminary structure-activity relationships (SAR). For the inhibitor library, three aspects of the hybrid analog were focused on. Firstly, driven by the slight rotation of the p-nitrobenzyl moiety, JH-ENT-02, JH-ENT-03, and JH-ENT-04 analogs were designed in an attempt to adjust the angle to mimic NBMPR. Secondly, the truncated analog JH-ENT-05 was devised to determine if ‘opportunistic site 1’ could be leveraged, while making the inhibitors smaller and simpler to synthesize. Lastly, an additional ribose unit was introduced to analogs JH-ENT-06 and JH-ENT-07 to take advantage of the additional interactions observed with the ribose unit of NBMPR. The synthesis of each of these inhibitors will be briefly discussed.

To synthesize JH-ENT-02 the phenol of ester 3.006 of JH-ENT-01 (3.010) was replaced with a thiol. This was accomplished by coupling dimethylthiocarbamoyl chloride (3.011) with 3,4-dimethoxy-5-hydroxybenzoic acid methyl ester (3.006) with the addition of NaH, followed by reflux in phenyl ether to provide the rearranged product 3.013. Finally, transesterification with NaOMe provides thiol 3.014 in 82% yield (Scheme 10).

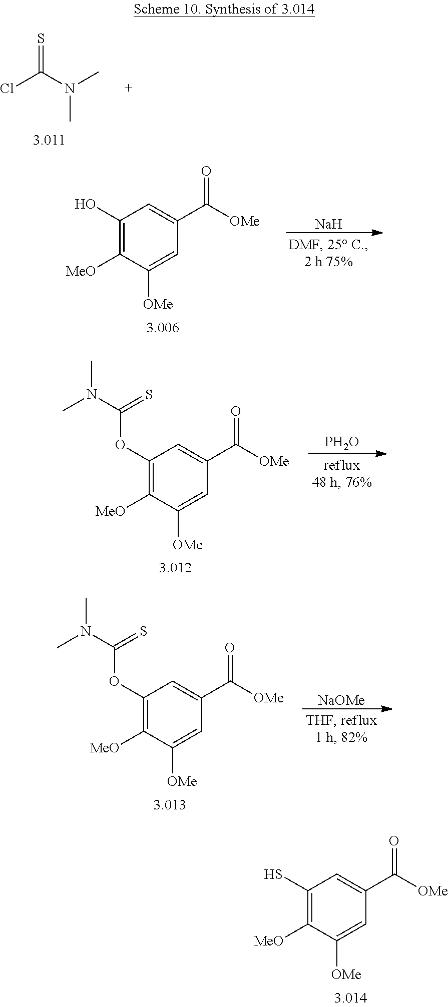

With the thiol (3.014) in hand, the same synthetic approach was pursued as JH-ENT-01. The p-nitrobenzyl moiety was introduced via reflux with thiol (3.014) under basic conditions and the resulting ester was hydrolyzed with 1 N LiOH to provide carboxylic acid 3.016. Next to introduce the alkyl chain, 3.016 was treated with thionyl chloride to form the acyl intermediate and then 3-bromo-1-propanol but unfortunately the conditions were too harsh and the p-nitrobenzyl group was removed and resulted in alkylation of the thiol (Scheme 11). As N,N′-dicyclohexylcarbodiimide (DCC) is a commonly used reagent for the formation of amide, peptide, or ester bonds, 3.016 was treated with DCC, 3-bromo-1-propanol in CH₂Cl₂ and successfully introduced the alkyl chain to form intermediate 3.017. Finally, 3.017 was coupled to homopiperazine 3.005 to provide JH-ENT-02 in a 44% yield.

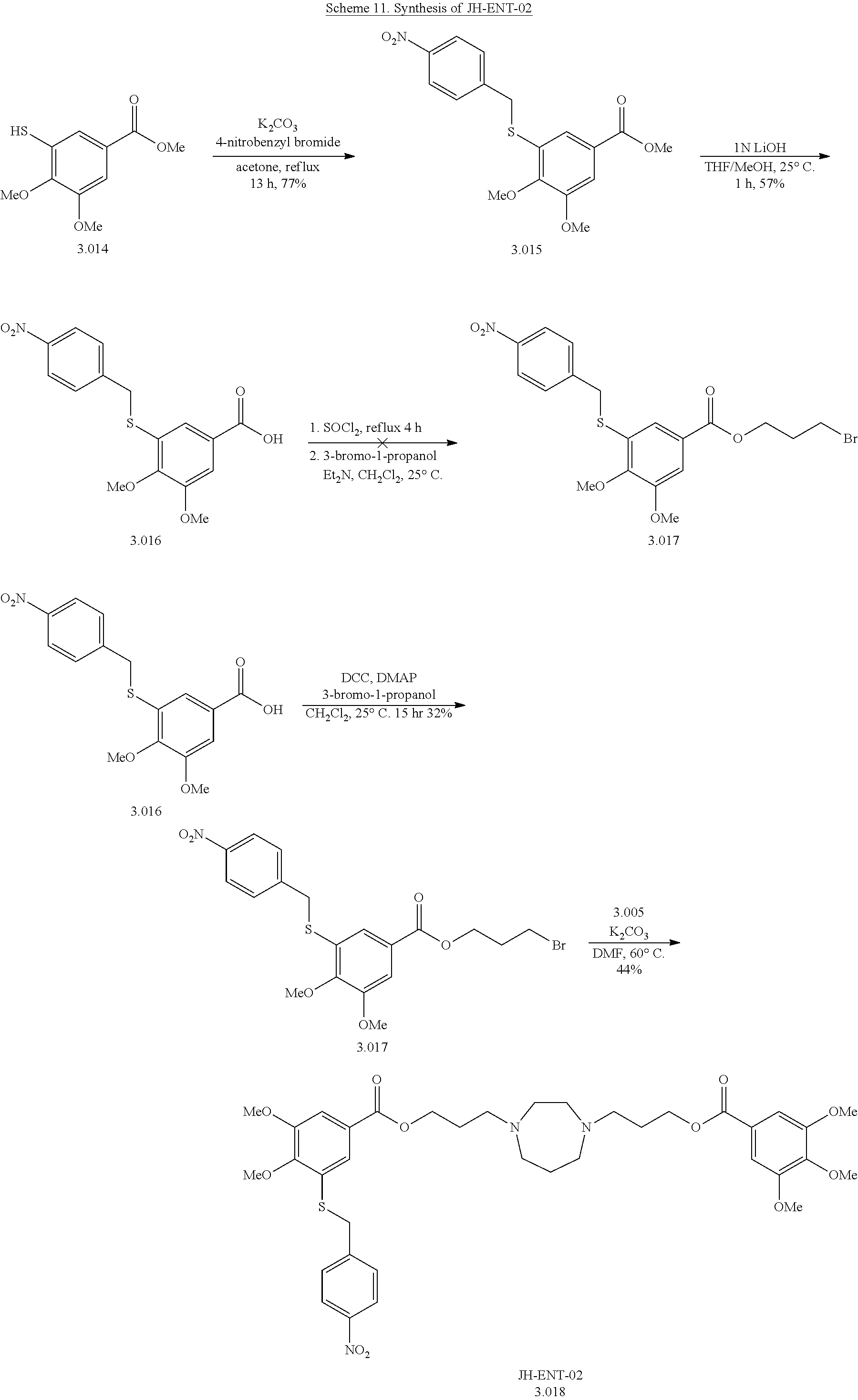

Next, attention was turned to the monomethyl analog JH-ENT-03. The synthesis began with the reduction of carboxylic acid 3.019 to the primary alcohol with BH3·DMS and B(OCH₃)₃, which was then converted to bromide 3.020 upon treatment with PBr₃ (Scheme 12). The remainder of the synthesis is carried out analogously to JH-ENT-02. Coupling to methyl ester 3.006, hydrolysis with 1 N LiOH, DCC coupling to 3-bromo-1-propanol and finally coupling to homopiperazine 3.005 were executed to complete the synthesis of JH-ENT-03 (Scheme 12).

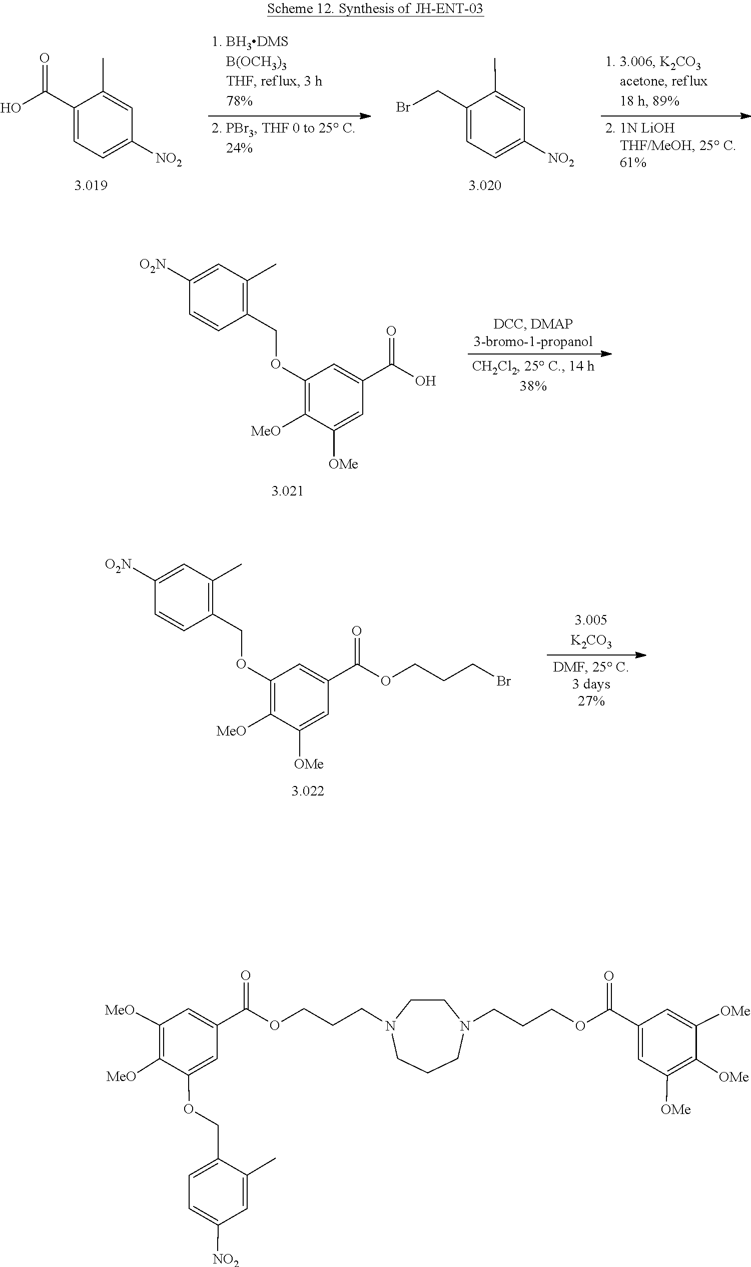 [SMILES: Cc1cc([N+](=O)[O-])ccc1C(=O)O ; Cc1cc([N+](=O)[O-])ccc1CBr ; COc1cc(C(=O)O)cc(OCc2ccc([N+](=O)[O-])cc2C)c1OC ; COc1cc(C(=O)OCCCBr)cc(OCc2ccc([N+](=O)[O-])cc2C)c1OC ; COc1cc(C(=O)OCCCN2CCCN(CCCOC(=O)c3cc(OC)c(OC)c(OCc4ccc([N+](=O)[O-])cc4C)c3)CC2)cc(OC)c1OC]

The introduction of the dimethyl nitrobenzyl moiety of JH-ENT-04 was slightly more complicated than the monomethyl analog. To construct the dimethyl nitrobenzyl bromide 3.030, the synthesis began by protecting 2,6-dimethylaniline with p-TsCl and then successively introduced the nitro moiety by treating 3.025 with NaNO₂ under reflux in AcOH, H₂O, and HNO₃ (Scheme 13). The tosyl protecting group was removed with concentrated sulfuric acid and the resulting free aniline was converted to nitrile 3.028 via a Sandmeyer reaction. 3.028 was then treated with DIBALH and 1 N HCl to form an aldehyde intermediate that was reduced by NaBH4 to provide primary alcohol 3.029.21 Finally, 3.029 was treated with PBr₃ to obtain bromide 3.030. Following the same synthetic approach as JH-ENT-02 & 03, the dimethyl analog JH-ENT-04 is obtained (Scheme 13).

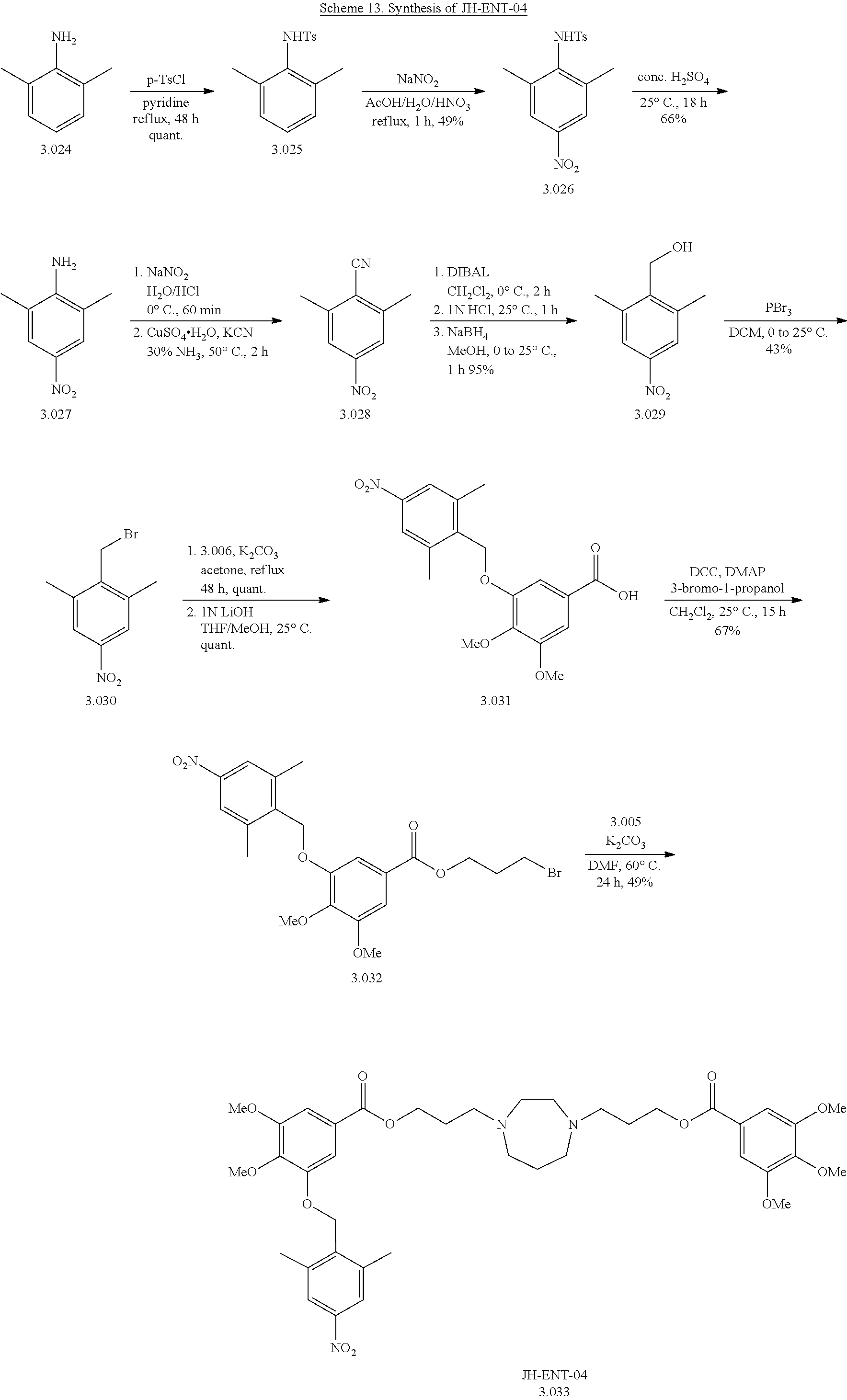

The simplest analog, JH-ENT-05, was attained by coupling intermediate 3.009 of JH-ENT-01 with 1-methylhomopiperazine under basic conditions in acetone in a 64% yield (Scheme 14).

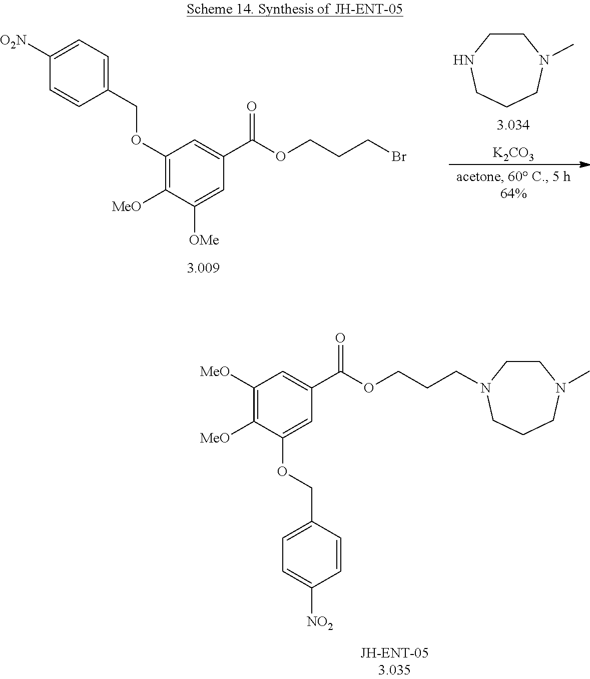

For the synthesis of JH-ENT-06 and JH-ENT-07, the synthesis began with the ribose moiety 3.039 (Scheme 15. Firstly, commercially available acetate 3.036 was converted to methyl ester 3.037 in 69% yield for 3 steps. 3.036 was treated with TMSCN and BF₃·OEt₂ to form a nitrile intermediate which was subsequently treated with NaOMe to form the methyl ester and simultaneously remove the benzoyl protecting groups. The resulting syn-diol was then protected with 2,2-dimethoxypropane in the presence of p-TsOH. Next, the primary alcohol was protected with TBDPSCl to provide 3.038. Finally, ester reduction with NaBH₄ and tosylation provides the ribose moiety 3.039 (Scheme 15).

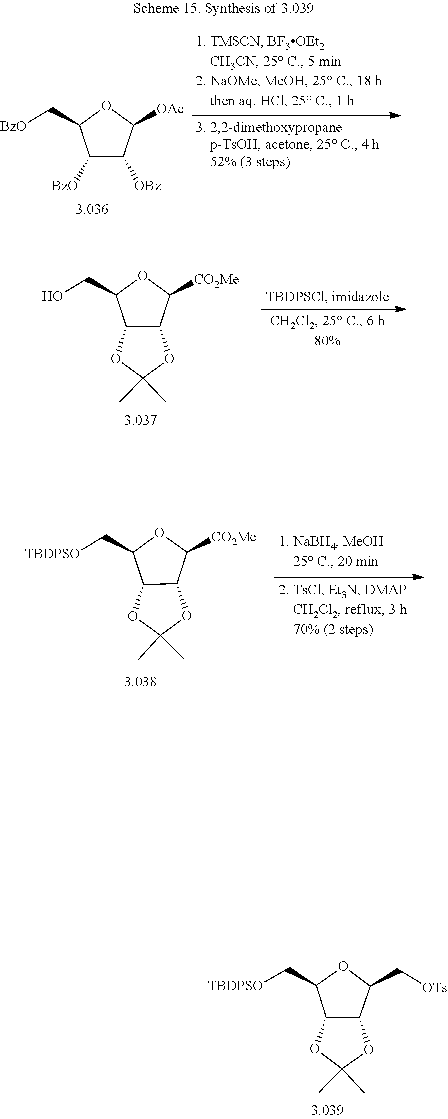

To introduce the ribose moiety to the substrate, methyl 3,4,5-trihydroxybenzoate 3.040 was treated with Mel and K₂CO₃ to give the para-methylated phenol in a 60% yield (Scheme 16). From there, ribose moiety 3.039 was introduced under basic conditions in DMF warmed to 40° C. over 3 days. Next, p-nitrobenzyl bromide was coupled to phenol 3.042 to provide 3.043. The synthesis was completed as the other inhibitors, hydrolysis with 1 N LiOH, DCC coupling of 3-bromo-1-propanol and coupling to homopiperazine 3.005 but an additional deprotection step with TFA provides JH-ENT-06. For the final inhibitor, JH-ENT-07 was obtained by treating intermediate 3.043 with TFA (Scheme 17).

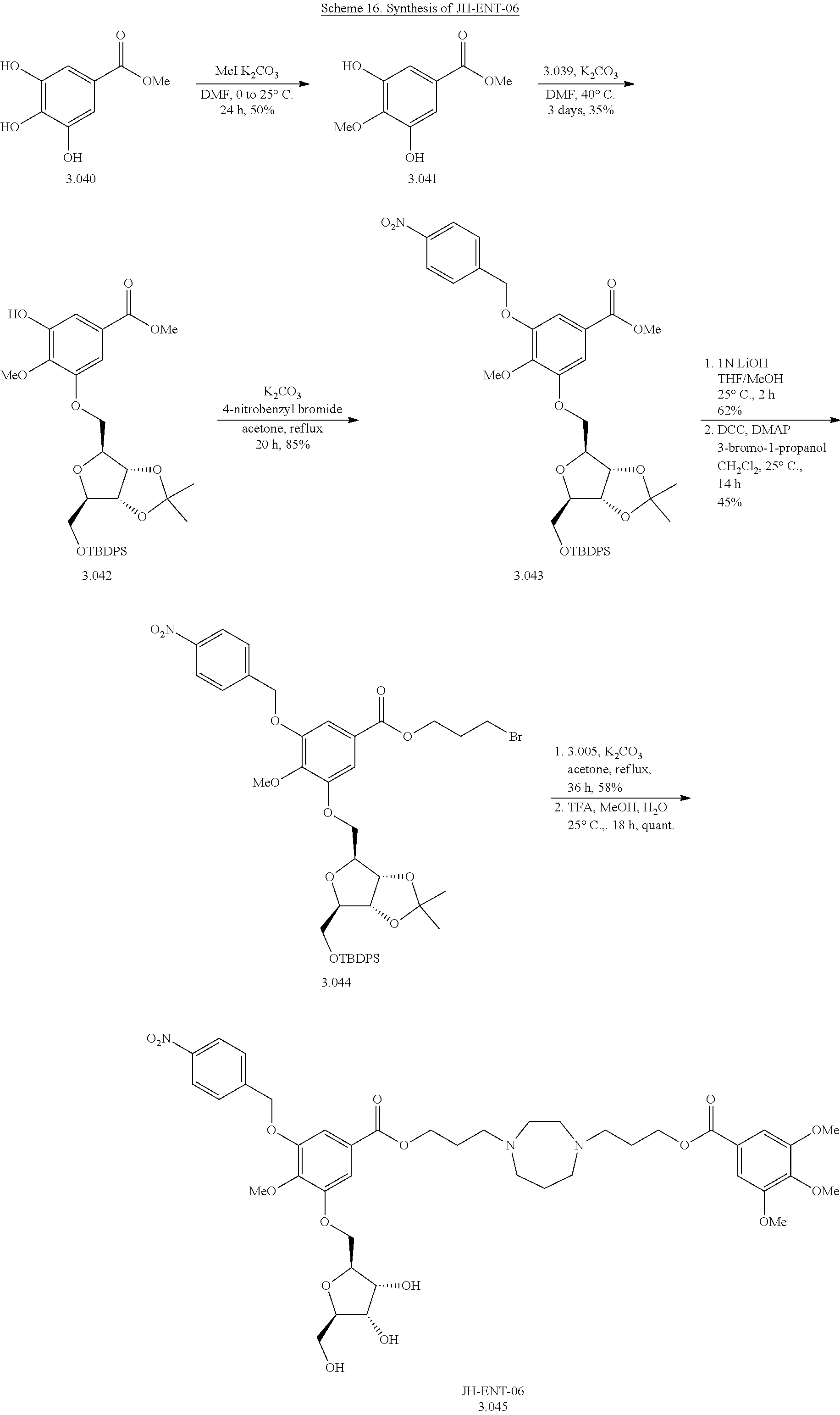

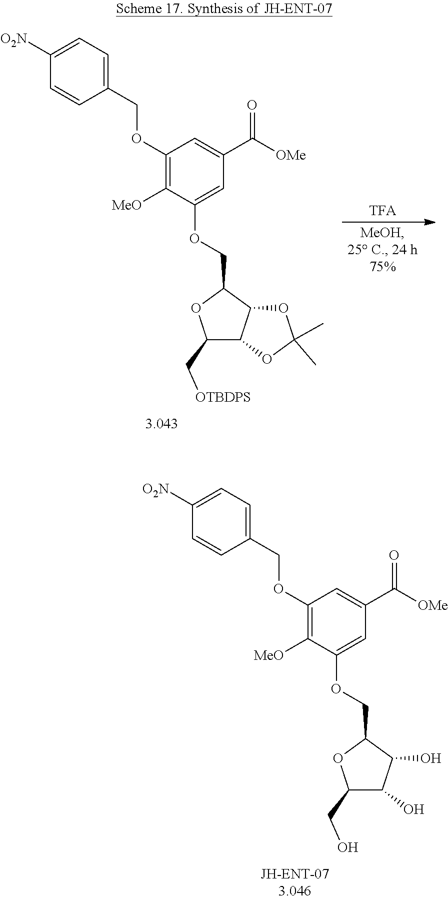

### Rational Design for Second Generation ENT1 Inhibitors

Motivated by the promising activity of the JH-ENT-07 inhibitor, expansion on this novel chemotype was sought for the development of more potent ENT1 inhibitors. To design novel compounds, the co-crystal structure of NBMPR in complex with hENT1 was utilized to study the binding pose of JH-ENT-07. Additional space at the top of the binding pocket was available for expansion off of the methyl ester of JH-ENT-07, as well as the potential for additional interactions with Gln158 at the bottom. Therefore, ˜150 compounds were rationally designed and evaluated using Maestro's Glide (Schrödinger). The top two hit compounds that exhibited excellent docking scores, maintained key interactions with Gln158 and Trp29 but additionally showed hydrogen bonding with Thr85 have been synthesized and are currently being evaluated for their bioactivity.

### Efforts Towards a Macrocyclic ENT Inhibitor

Regarding the second aim, improving the pharmacological properties of ENT inhibitors, it was hypothesized that a macrocyclic version of dilazep would achieve this goal by decreasing conformational flexibility, which will reduce entropy on binding, and increase selectivity and permeability of the inhibitor. Therefore, to determine the optimal linker length and stereochemistry of the macrocycle, a docking study (Maestro's Glide, Schrödinger) was performed, utilizing the crystal structure of ENT1 in complex with dilazep. This study demonstrated that a 3-carbon linker and R stereochemistry (highlighted in blue) were necessary to mimic the binding pose of dilazep.

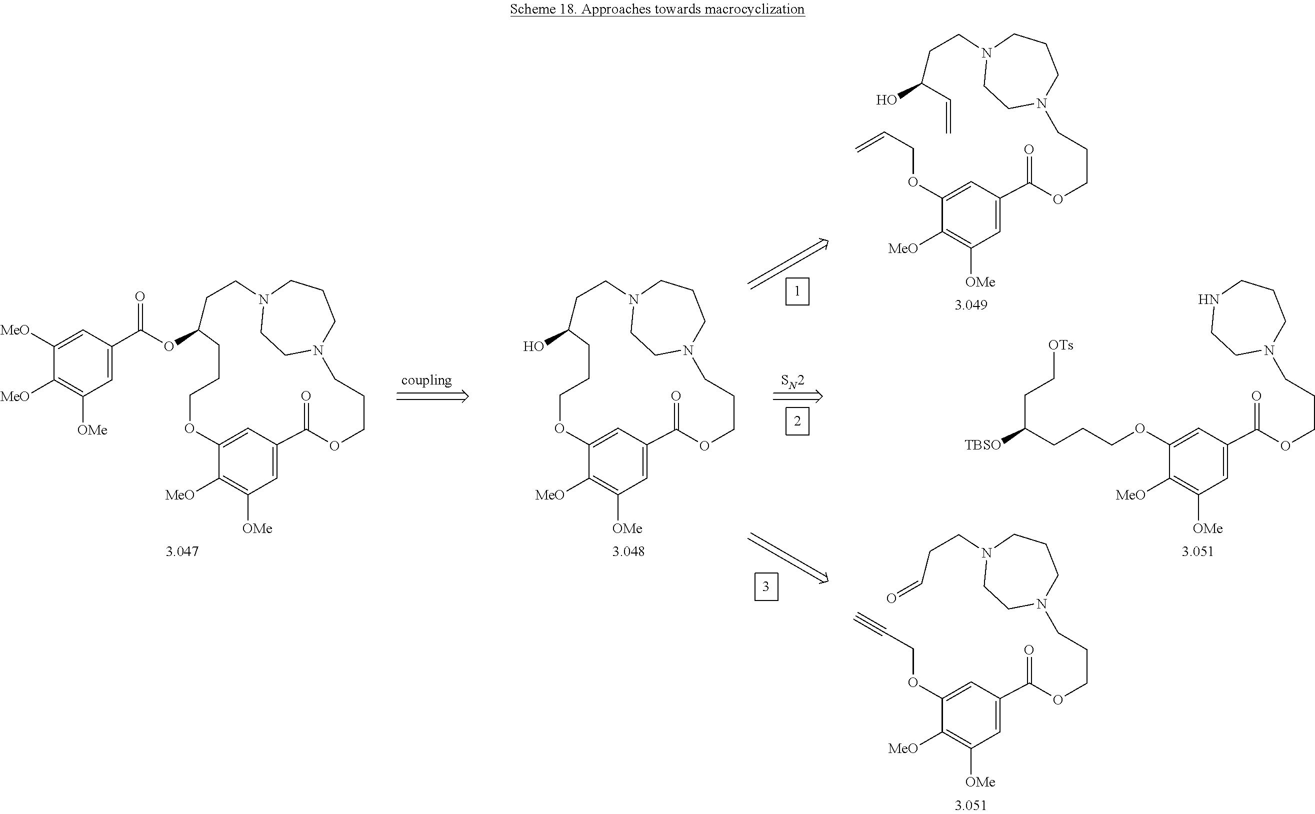 [SMILES: COc1cc(C(=O)O[C@@H]2CCCOc3cc(cc(OC)c3OC)C(=O)OCCCN3CCCN(CC2)CC3)cc(OC)c1OC ; COc1cc2cc(c1OC)OCCC[C@@H](O)CCN1CCCN(CCCOC2=O)CC1 ; C=CCOc1cc(C(=O)OCCCN2CCCN(CC[C@H](O)C=C)CC2)cc(OC)c1OC ; COc1cc(C(=O)OCCCN2CCCNCC2)cc(OCCC[C@H](CCOS(=O)(=O)c2ccc(C)cc2)O[Si](C)(C)C(C)(C)C)c1OC ; C#CCOc1cc(C(=O)OCCCN2CCCN(CCC=O)CC2)cc(OC)c1OC]

### First Approach Towards Macrocyclization

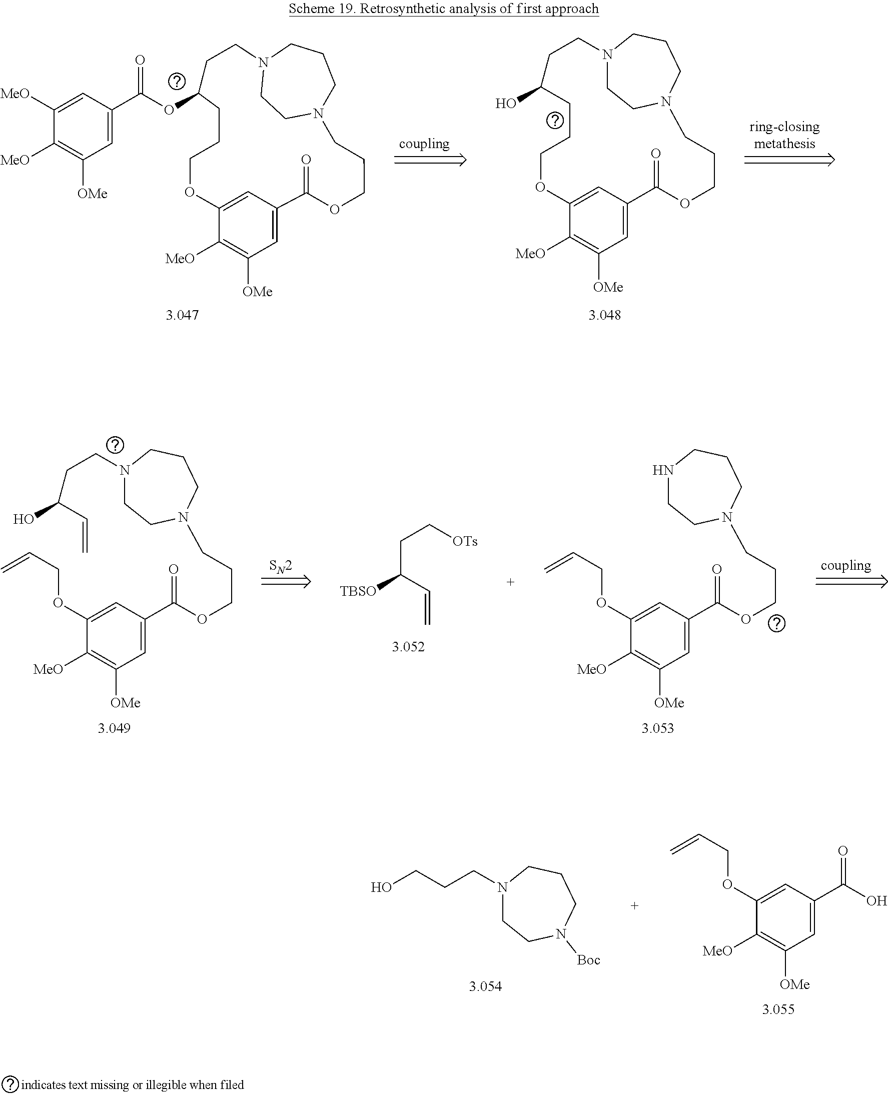

Initial efforts began with the RCM approach (Scheme 19). It was anticipated that the substrate for RCM could be obtained by coupling easily synthesizable building blocks 3.054 and 3.055 to provide homopiperazine 3.053, which can be further coupled to tosylate 3.052 through an SN²-type reaction. Therefore, 3.055 was synthesized via coupling of phenol 3.006 with allyl bromide, followed by hydrolysis of the methyl ester with 1 N LiOH (Scheme 20). Next, homopiperazine 3.053 was obtained by coupling 1-Boc-homopiperazine with 3-bromo-1-propanol. The resulting primary alcohol was coupled to the carboxylic acid of 3.055 via DCC coupling in the presence of DMAP. Finally, the Boc protecting group was removed quantitatively upon treatment with TFA.

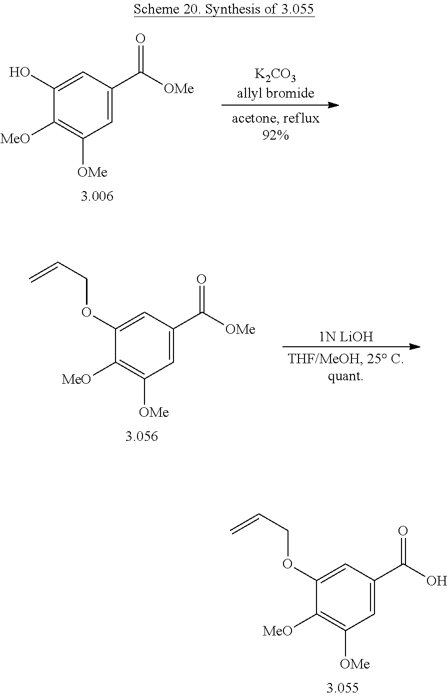 [SMILES: COC(=O)c1cc(O)c(OC)c(OC)c1 ; "K2CO3allyl bromide" ; C=CCOc1cc(C(=O)OC)cc(OC)c1OC ; "1N LiOH" ; C=CCOc1cc(C(=O)O)cc(OC)c1OC]

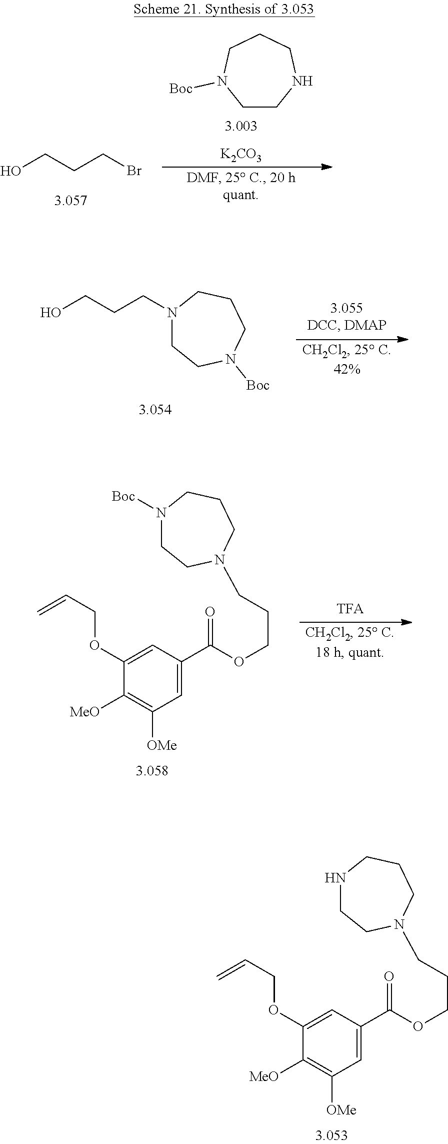

The synthesis of 3.052 began by coupling t-butylacetate to ethyl 3-chloropropionate via in situ generation of LDA, followed by elimination of the resulting chloride with Et₃N to provide 3.060. Next, the ketone was reduced with NaBH₄ to provide an enantiomeric mixture of 3.061 in 25% yield over three steps. The enantiomers were then separated using enzymatic resolution with vinyl acetate and amano lipase to provide the desired S acetate in 40% yield. The acetate was then removed with K₂CO₃, and the resulting secondary alcohol was protected with TBSCl to provide 3.064.

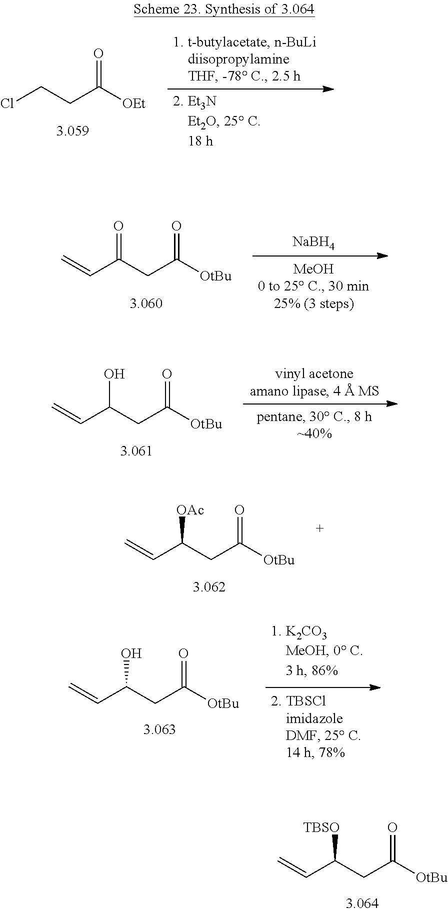

To determine the stereoselectivity of the enzymatic resolution with amano lipase, Mosher's acid chloride (3.067) was utilized to generate diastereomers 3.068 and 3.069 (Scheme 24). The ¹H NMR spectrum (FIG. 38) was compared to racemic Mosher ester 3.070 (FIG. 37). Looking at the racemic mixture, two sets of methoxy peaks are clearly observed around 3.5 ppm and alkyl peaks further shifted upfield from 2.5 ppm. For the enantio-enriched material no peaks are observed upfield of 2.5 ppm and see a ratio of 30:1 for the methoxy peaks. Therefore, the enzymatic resolution provided ≥95% e.e. for the desired secondary alcohol.

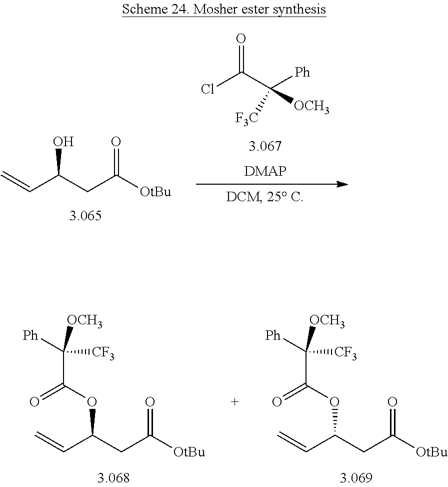 [SMILES: CO[C@@](C(=O)Cl)(c1ccccc1)C(F)(F)F ; C=C[C@@H](O)CC(=O)OC(C)(C)C ; C=C[C@H](CC(=O)OC(C)(C)C)OC(=O)[C@](OC)(c1ccccc1)C(F)(F)F ; C=C[C@@H](CC(=O)OC(C)(C)C)OC(=O)[C@](OC)(c1ccccc1)C(F)(F)F]

To conserve material, the racemic form of the ester was utilized for the attempts at the RCM reaction. Moving forward, ester 3.071 was reduced with DIBAL to the primary alcohol, which was then converted to tosylate 3.072 by treatment with p-TsCl in the presence of Et₃N. Tosylate 3.072 was then coupled to homopiperazine 3.053 under refluxing conditions with K₂CO₃ to provide 3.073, the substrate for RCM. Subsequently, 3.073 was treated with Grubbs II (20 mol %) in very dilute CH₂Cl₂ (1 mM) under reflux. After 14 h, several new spots on TLC were observed in addition to the starting material but due to limited material and tight separation, instead of purification, the crude material was treated with TBAF to remove the TBS protecting group to provide 3.075. Although the small scale prevented purification and isolation, HRMS confirmed the formation of 3.075.

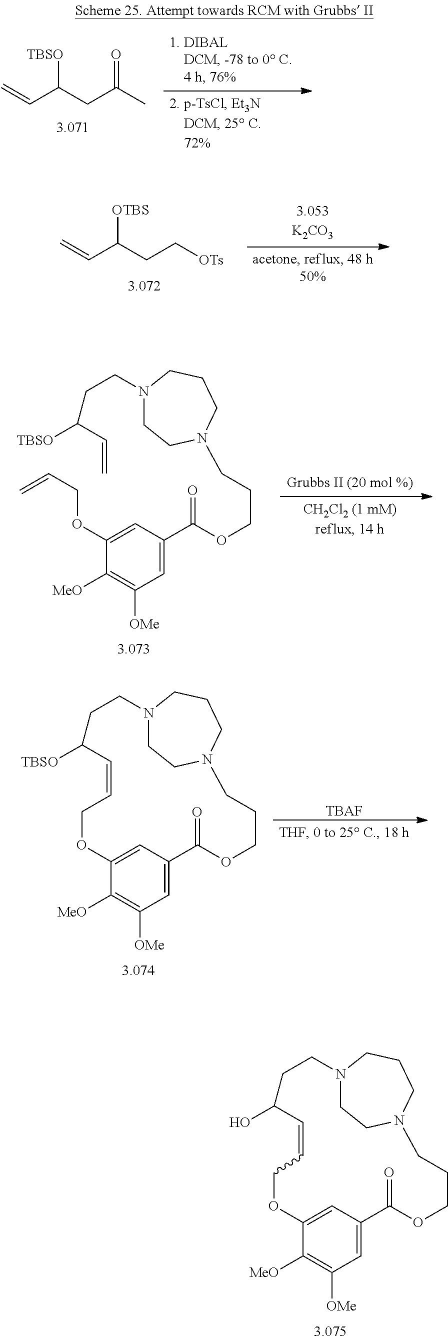 [SMILES: C=CC(CC(C)=O)O[Si](C)(C)C(C)(C)C ; C=CC(CCOS(=O)(=O)c1ccc(C)cc1)O[Si](C)(C)C(C)(C)C ; C=CCOc1cc(C(=O)OCCCN2CCCN(CCC(C=C)O[Si](C)(C)C(C)(C)C)CC2)cc(OC)c1OC ; COc1cc2cc(c1OC)OC/C=C\C(O[Si](C)(C)C(C)(C)C)CCN1CCCN(CCCOC2=O)CC1 ; "TBAF" ; COc1cc2cc(c1OC)OCC=CC(O)CCN1CCCN(CCCOC2=O)CC1]

Encouraged by these results, additional attempts for the RCM reaction were attempted in CH₂Cl₂ and benzene (Table 3). Unfortunately, the initial attempt was not reproduced and mostly starting material was obtained from these trials. To determine if this approach is applicable for the formation of the macrocycle several modifications should be attempted, including, exploring other variations of the Grubb's catalyst as well as varying the concentration of the reaction. Very dilute (1 mM) concentrations were used to promote intramolecular olefin metathesis but by increasing the concentration the reaction may be pushed forward and the starting material may be consumed.

TABLE 3
Attempts Towards Cyclization with Grubbs II

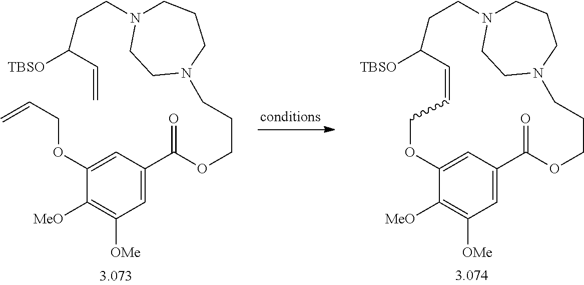 [SMILES: C=CCOc1cc(C(=O)OCCCN2CCCN(CCC(C=C)O[Si](C)(C)C(C)(C)C)CC2)cc(OC)c1OC ; COc1cc2cc(c1OC)OCC=CC(O[Si](C)(C)C(C)(C)C)CCN1CCCN(CCCOC2=O)CC1]

| Entry | Conditions | Result |
|---|---|---|
| 1 | Grubbs II (25 mol %), CH₂Cl₂, reflux, 48 h | 3.073 |
| 2 | Grubbs II (25 mol %), benzene, reflux, 48 h | 3.073 |

### Second Approach Towards Macrocyclization

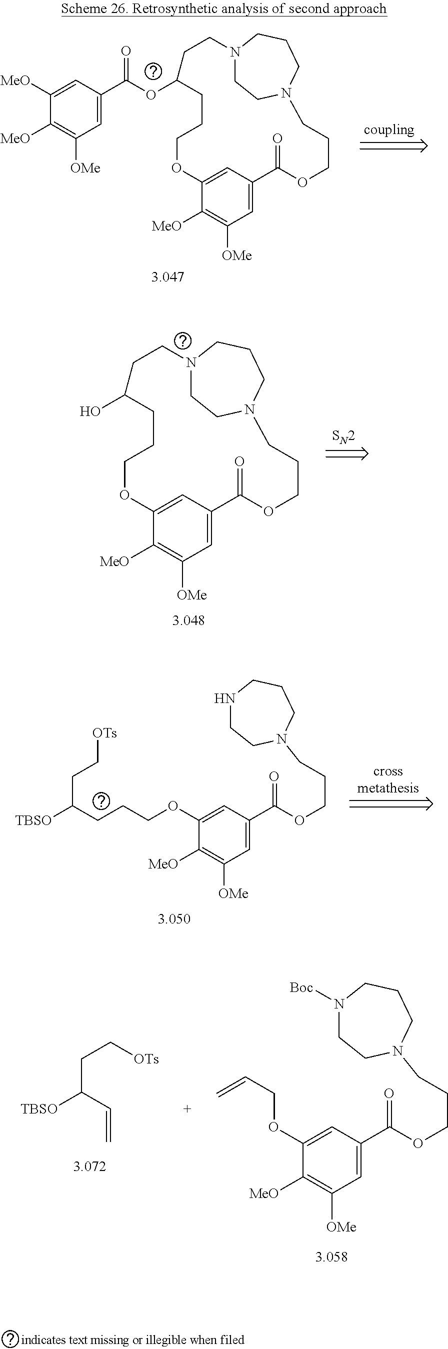 [SMILES: COc1cc(C(=O)OC2CCCOc3cc(cc(OC)c3OC)C(=O)OCCCN3CCCN(CC2)CC3)cc(OC)c1OC ; COc1cc2cc(c1OC)OCCCC(O)CCN1CCCN(CCCOC2=O)CC1 ; COc1cc(C(=O)OCCCN2CCCNCC2)cc(OCCCC(CCOS(=O)(=O)c2ccc(C)cc2)O[Si](C)(C)C(C)(C)C)c1OC ; C=CC(CCOS(=O)(=O)c1ccc(C)cc1)O[Si](C)(C)C(C)(C)C ; C=CCOc1cc(C(=O)OCCCN2CCCN(C(=O)OC(C)(C)C)CC2)cc(OC)c1OC]

With limited success with RCM, a second approach was attempted that relied on macrocyclization via SN² with homopiperazine 3.050. It was postulated that the macrocyclization substrate 3.050 could be obtained by cross-metathesis (CM) of previously synthesized intermediates 3.072 and 3.058 (Scheme 26). Therefore, 3.072 and 3.058 were subjected to CM conditions with Grubbs II (Table 4) but unfortunately, the terminal olefin of 3.058 was simply isomerized to produce a mixture of cis and trans alkene in a 1:2.5 ratio, respectively (3.077). No CM product was observed.

TABLE 4
CM attempts with 3.072 and 3.058

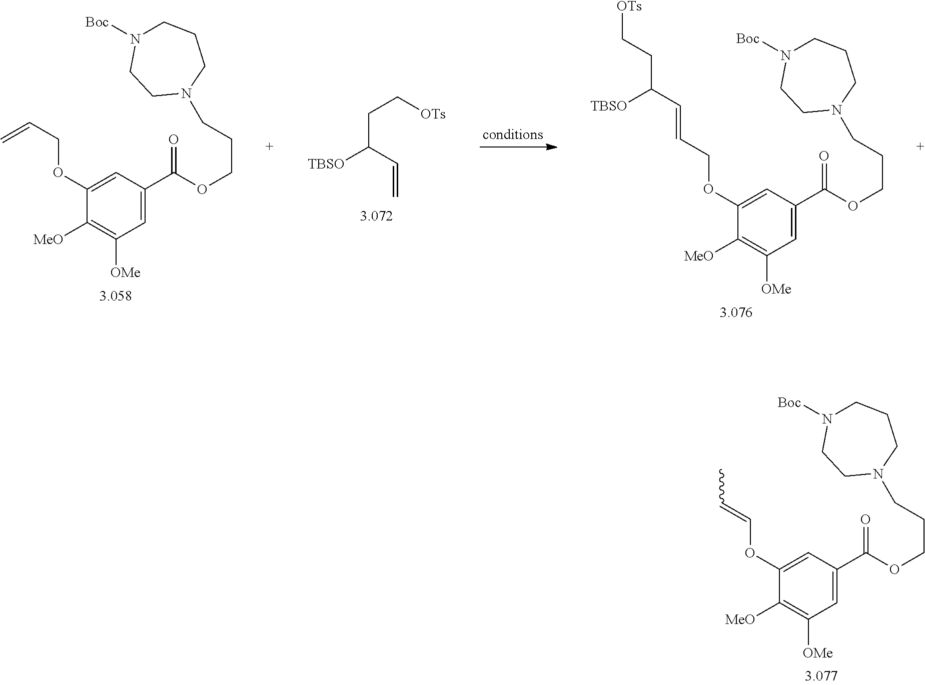

| Entry | Conditions | Result |
|---|---|---|
| 1 | Grubbs II (25 mol %), CH₂Cl₂, reflux, 48 h | 3.077 |
| 2 | Grubbs II (20 mol %), benzene, reflux, 48 h | 3.077 |

At this stage, the approach was revised towards intermediate 3.050 with the intention to extend 3.072 first and then couple to phenol to provide the macrocyclization substrate 3.050. Initially, attempts were made to build the alkyl chain through CM with cis-1,4-diacetoxy-2-butene (3.078) and substrates (3.072 and 3.071), but the coupling reactions were unsuccessful and the only newly formed product was trans-1,4-diacetoxy-2-butene (3.080) (Scheme 27).

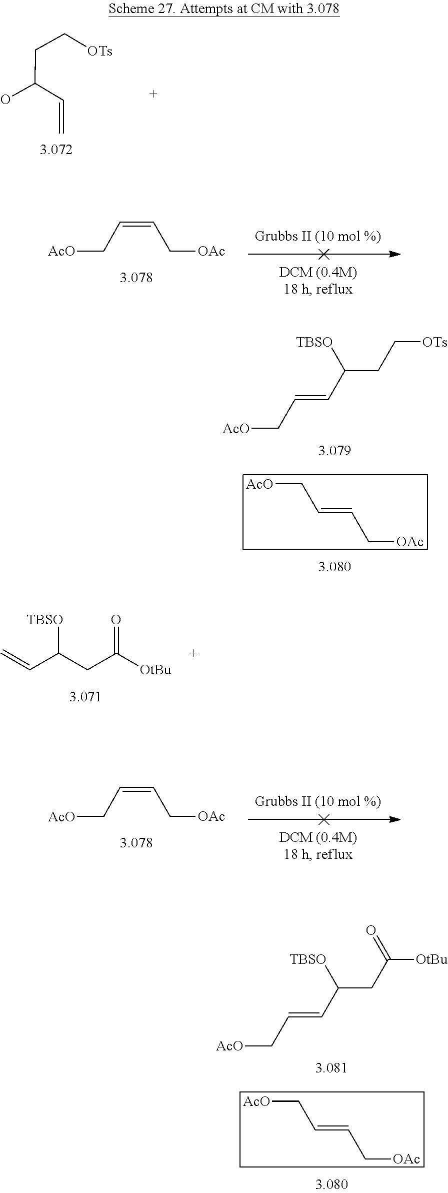

As CM had proven difficult with the substrate, other options began to be explored for extending the alkyl chain and started with a Horner-Wadsworth-Emmons (HWE) reaction. First, t-butyl ester 3.071 was reduced with DIBAL to provide a primary alcohol which was subsequently protected with BnBr. Next, alkene 3.082 was oxidized to an aldehyde via ozonolysis in the presence of PPh₃ and immediately reacted with Ph₃PCHCO₂Et to provide the HWE product 3.083 in 10% for two steps.

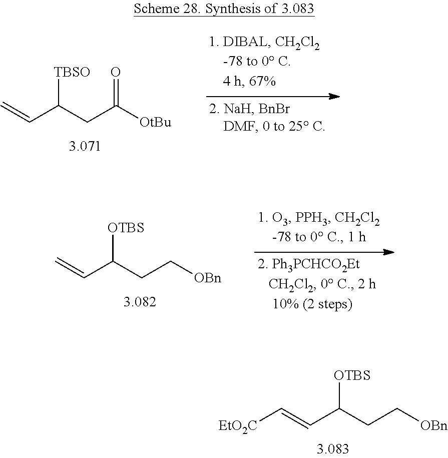 [SMILES: C=CC(CC(=O)OC(C)(C)C)O[Si](C)(C)C(C)(C)C ; C=CC(CCOCc1ccccc1)O[Si](C)(C)C(C)(C)C ; CCOC(=O)/C=C/C(CCOCc1ccccc1)O[Si](C)(C)C(C)(C)C]

Although the HWE product was obtained via ozonolysis, unacceptably low yields pushed us to seek out another route for oxidation. Consequently, the aldehyde necessary for the HWE reaction was synthesized via oxidative cleavage with OSO₄, NMO, and NaIO₄. To apply this approach, t-butyl ester 3.061 was reduced with LAH and selectively protected the primary alcohol with trityl chloride (TrCl) and subsequent benzyl protection of the secondary alcohol (Scheme 29). Intermediate 3.085 was then treated with OsO₄ and N-methylmorpholine N-oxide (NMO), followed by NaIO₄ to form an aldehyde intermediate which was immediately subjected to HWE with Ph₃PCHCO₂Et to provide 3.086 in 49% for three steps. Next, the ethyl ester was reduced with DIBAL and activating the newly formed alcohol with MsCl to provide substrate 3.087 primed for coupling with the phenol.

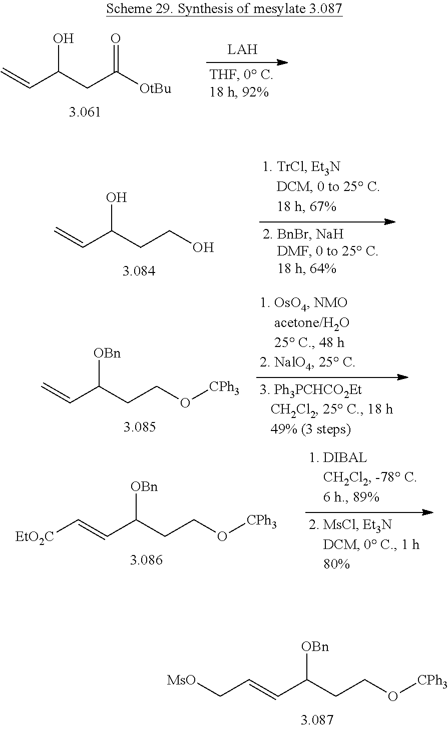

Allyl deprotection of 3.058 with Pd(Ph₃P)₄ and dimedone in THE provided phenol 3.088, which was then coupled to mesylate 3.087 upon treatment with K₂CO₃ in acetone. The trityl protecting group was selectively removed in the presence of Boc utilizing dichloroacetic acid. The primary alcohol was then converted to mesylate 3.090 and the Boc group was removed with TFA. Crude homopiperazine was subjected to K₂CO₃ in acetone or DMF but regrettably, no cyclized product was obtained. Instead, starting material was recovered or the material decomposed. Like the first approach, additional efforts would be required to determine the applicability of this approach towards macrocyclization. Many different changes could be made to optimize this reaction, such as, varying the base, solvent, and reaction temperature.

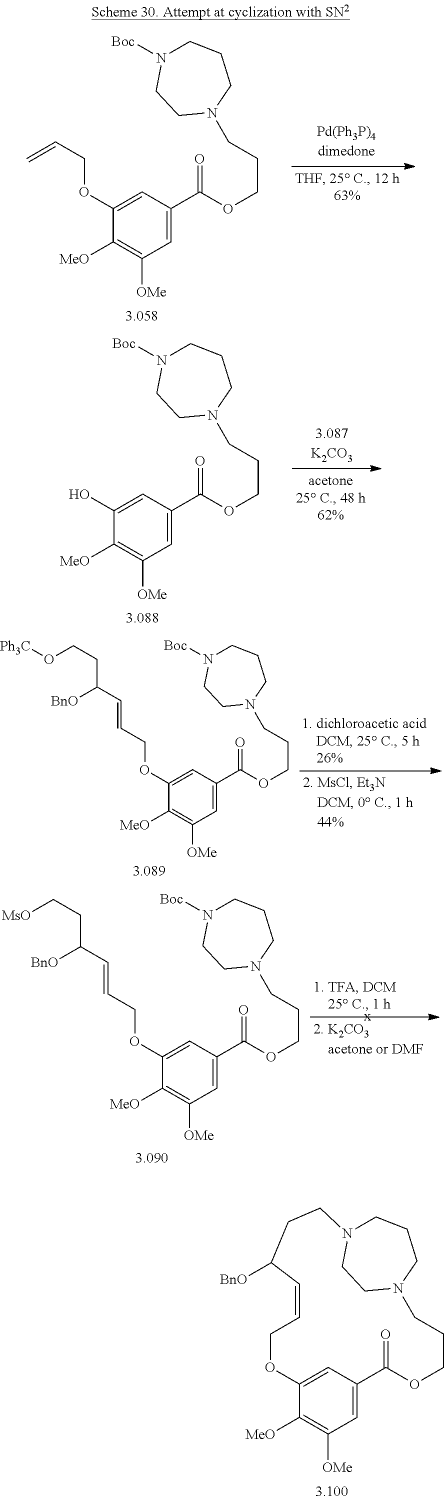

### Third Approach Towards Macrocyclization

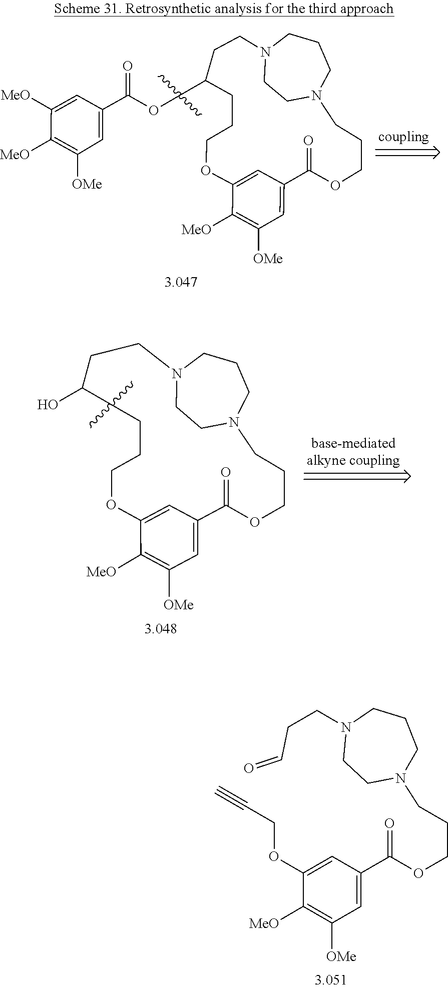

Although additional work needs to be done to determine the viability of the SN² reaction, the third approach was attempted. It was anticipated that alkyne intermediate 3.051 may be more reactive and therefore more conducive towards cyclization. To obtain alkynyl intermediate 3.051, the synthesis began by propargylation of phenol 3.006, followed by hydrolysis and DCC coupling of the resulting carboxylic acid. Following deprotection with TFA, the homopiperazine was coupled to 3-bromo-1-propanol to provide intermediate 3.103.

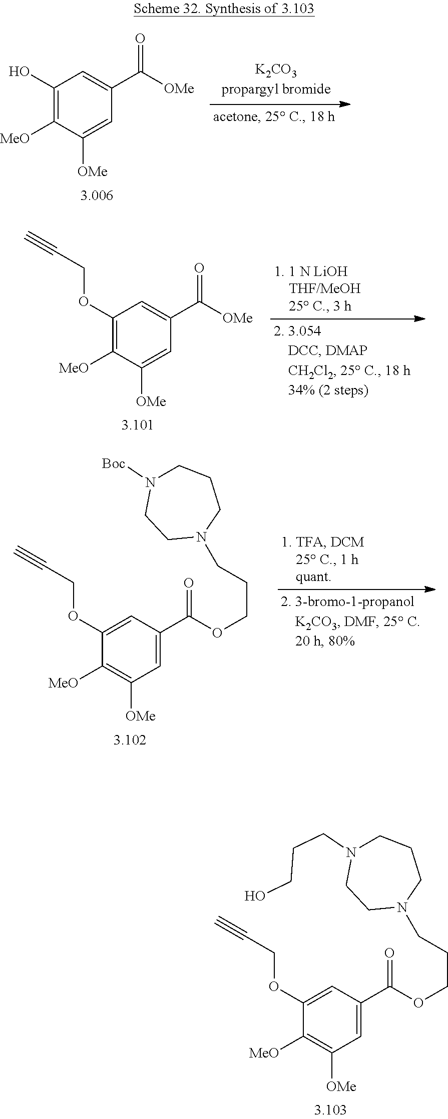 [SMILES: COC(=O)c1cc(O)c(OC)c(OC)c1 ; C#CCOc1cc(C(=O)OC)cc(OC)c1OC ; C#CCOc1cc(C(=O)OCCCN2CCCN(C(=O)OC(C)(C)C)CC2)cc(OC)c1OC ; C#CCOc1cc(C(=O)OCCCN2CCCN(CCCO)CC2)cc(OC)c1OC]

With the primary alcohol in hand, oxidation was first attempted with PCC. (Table 5). Unfortunately, the material decomposed. Next, oxidation was attempted with Dess-Martin periodinane (DMP) but unfortunately, this also resulted in decomposition. Finally, Swern oxidation was attempted but starting material was recovered. While additional efforts can be made for the oxidation of 3.103, the use of a Weinreb amide will likely provide a more stable intermediate and allow us to attempt the macrocyclization under basic conditions (Scheme 33).

TABLE 5
Attempts for Oxidation of 3.103

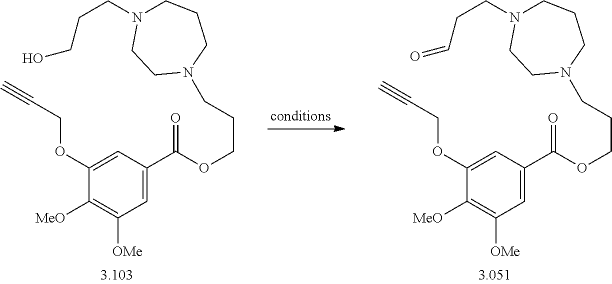 [SMILES: C#CCOc1cc(C(=O)OCCCN2CCCN(CCCO)CC2)cc(OC)c1OC ; C#CCOc1cc(C(=O)OCCCN2CCCN(CCC=O)CC2)cc(OC)c1OC]

| Entry | Conditions | Result |
|---|---|---|
| 1 | PCC, CH₂Cl₂, 25° C., 1 h | decomposed |
| 2 | DMP, CH₂Cl₂, 25° C., 1 h | decomposed |
| 3 | oxalyl chloride, DMSO, Et₃N, CH₂Cl₂, −—78° C., 2 h | 3.103 |

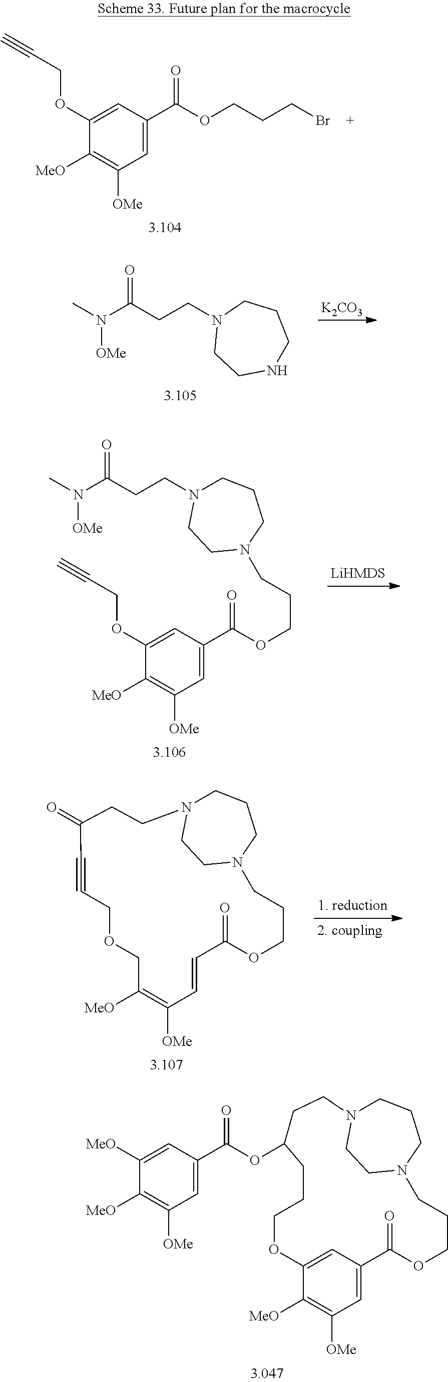

### General Methods

All reactions were conducted in oven-dried glassware under nitrogen or argon. Unless otherwise stated all reagents were purchased from commercial suppliers and used without further purification. All solvents were American Chemical Society (ACS) grade or better and used without further purification except tetrahydrofuran (THF), which was freshly distilled from sodium/benzophenone each time before use. Analytical thin layer chromatography (TLC) was performed with glass backed silica gel (60 Å) plates with fluorescent indication (Whatman). Visualization was accomplished by UV irradiation at 254 nm, or by staining with p-anisaldehyde solution or potassium permanganate solution followed by heating. Flash column chromatography was performed by using silica gel (particle size 230-400 mesh, 60 Å). All ¹H NMR and ¹³C NMR spectra were recorded with a Varian 400 (400 MHz) and a Bruker 500 (500 MHz) spectrometer. All NMR δ values are given in parts per million (ppm) and are referenced to the residual isotopomer solvent signals (CDCl₃: δ=7.26 ppm, CD₃OD: δ=3.31 ppm) for 1H NMR spectra, or the solvent signals for ¹³C spectra. Coupling constants (J) are given in Hertz (Hz) and multiplicities are indicated using the conventional abbreviation (s=singlet, d=doublet, t=triplet, q=quartet, m=multiplet or overlap of non-equivalent resonances, br=broad). Electrospray ionization (ESI) mass spectrometry (MS) was recorded with an Agilent 1100 series (LC/MSD trap) spectrometer and performed to obtain the molecular masses of the compounds. Infrared (IR) absorption spectra were determined with a Thermo-Fisher (Nicolet iS50) spectrometer.

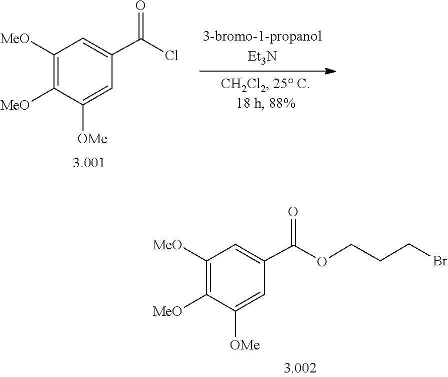 [SMILES: COc1cc(C(=O)Cl)cc(OC)c1OC ; COc1cc(C(=O)OCCCBr)cc(OC)c1OC]

To a solution of 3,4,5-trimethoxybenzoyl chloride (350 mg, 1.52 mmol) in CH₂Cl₂ (2.5 mL) was added 3-bromo-1-propanol (0.15 mL, 1.67 mmol) and Et3N (0.5 mL) at 25° C. After stirring for 18 h, the reaction was quenched with H₂O and the resulting mixture was diluted with CH₂Cl₂. The layers were separated, and the aqueous layer was extracted with CH₂Cl₂. The combined organic layers were dried over anhydrous anhydrous Na₂SO₄, filtered, and concentrated in vacuo. The residue was purified by column chromatography (silica gel, hexanes/EtOAc, 4/1) to afford 3.002 (432 mg, 88%). ¹H NMR (500 MHz, CDCl₃) δ 7.29 (s, 2H), 4.47 (t, J=6.1 Hz, 2H), 3.91 (s, 9H), 3.54 (t, J=6.5 Hz, 2H), 2.34 (dt, J=6.3, 6.3 Hz, 2H).

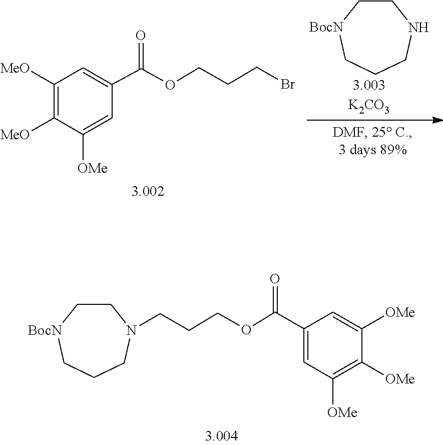 [SMILES: COc1cc(C(=O)OCCCBr)cc(OC)c1OC ; CC(C)(C)OC(=O)N1CCCNCC1 ; COc1cc(C(=O)OCCCN2CCCN(C(=O)OC(C)(C)C)CC2)cc(OC)c1OC]

To a solution of 3.002 (136 mg, 0.42 mmol) in DMF (1.5 mL) was added 1-Boc-homopiperazine (0.11 mL, 0.84 mmol) and K₂CO₃ (582 mg, 4.21 mmol) at 0° C. After stirring for 3 days at 25° C., the reaction was quenched with the addition of H₂O and the resulting mixture was diluted with EtOAc. The layers were separated, and the aqueous layer was extracted with EtOAc. The combined organic layers dried over anhydrous Na₂SO₄, filtered, and concentrated in vacuo. The residue was purified by column chromatography (silica gel, CH₂Cl₂/MeOH, 50/1) to afford 3.005 (169 mg, 89%). ¹H NMR (500 MHz, CDCl₃) δ 7.28 (s, 2H), 4.40 (t, J=6.4 Hz, 2H), 4.27-4.23 (m, 2H), 3.90 (s, 6H), 3.90 (s, 3H), 3.52-3.32 (m, 8H), 2.13 (dt, J=6.3, 6.2 Hz, 2H), 1.86-1.82 (m, 2H), 1.44 (s, 9H).

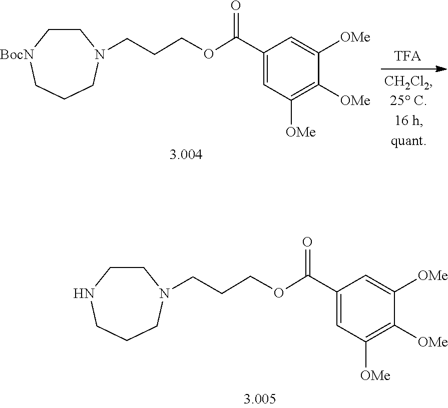 [SMILES: COc1cc(C(=O)OCCCN2CCCN(C(=O)OC(C)(C)C)CC2)cc(OC)c1OC ; COc1cc(C(=O)OCCCN2CCCNCC2)cc(OC)c1OC]

To a solution of 3.004 (72 mg, 0.16 mmol) in CH₂Cl₂ (1 mL) was added TFA (1 mL) at 25° C. After stirring for 16 h, the reaction was concentrated in vacuo and the crude material was used without further purification.

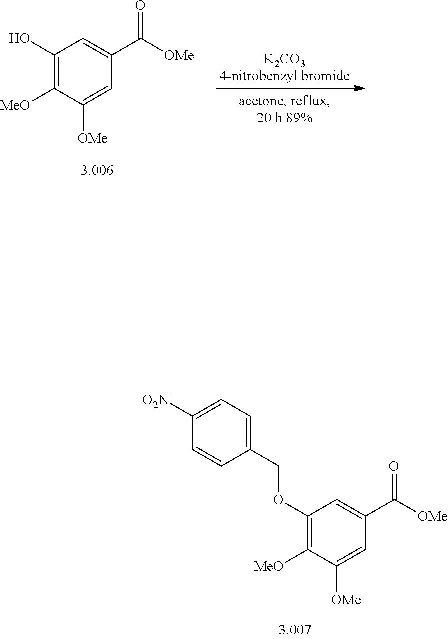 [SMILES: COC(=O)c1cc(O)c(OC)c(OC)c1 ; "K2CO34-nitrobenzyl bromide" ; COC(=O)c1cc(OC)c(OC)c(OCc2ccc([N+](=O)[O-])cc2)c1]

To a solution of 3,4-dimethoxy-5-hydroxybenzoic acid methyl ester (50 mg, 0.24 mmol) in acetone (1 mL) was added 4-nitrobenzylbromide (76 mg, 0.35 mmol) and K₂CO₃ (52 mg, 0.38 mmol) at 25° C. The reaction mixture was then heated to reflux for 20 h. Upon cooling to 0° C., the reaction was quenched with an addition of saturated aqueous NH₄Cl and the resulting mixture was diluted with EtOAc. The layers were separated, and the aqueous layer was extracted with EtOAc. The combined organic layers were dried over anhydrous Na₂SO₄, filtered, and concentrated in vacuo. The residue was purified by column chromatography (silica gel, hexanes/CH₂Cl₂/EtOAc, 5/1/1) to afford 3.007 (73 mg, 89%). ¹H NMR (400 MHz, CDCl₃) δ 8.24 (d, J=8.6 Hz, 2H), 7.63 (d, J=8.5 Hz, 2H), 7.33 (d, J=1.5 Hz, 1H), 7.30 (d, J=1.7 Hz, 1H), 3.93 (s, 3H), 3.91 (s, 3H), 3.89 (s, 3H).

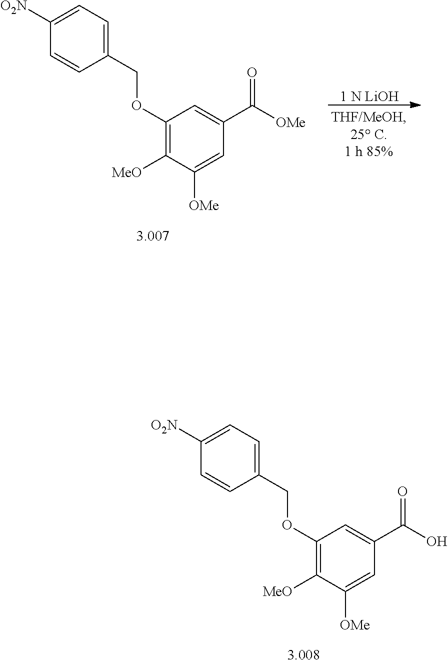 [SMILES: COC(=O)c1cc(OC)c(OC)c(OCc2ccc([N+](=O)[O-])cc2)c1 ; "1 N LiOH" ; COc1cc(C(=O)O)cc(OCc2ccc([N+](=O)[O-])cc2)c1OC]

To a solution of 3.007 (42 mg, 0.12 mmol), in THF/MeOH (3/1) (4 mL) was added 1 N LiOH (1 mL) at 25° C. After stirring for 1 h, 1 M HCl was added until the solution became acidic and the resulting mixture was diluted with EtOAc. The layers were separated, and the aqueous layer was extracted with EtOAc. The combined organic layers were dried over anhydrous Na₂SO₄, filtered, and concentrated in vacuo. The residue was purified by column chromatography (silica gel, CH₂Cl₂/MeOH, 10/1) to afford 3.008 (34 mg, 85%). ¹H NMR (400 MHz, CDCl₃) δ 8.26 (d, J=7.3 Hz, 2H), 7.64 (d, J=7.7 Hz, 2H), 7.40 (s, 1H), 7.36 (s, 1H), 3.96 (s, 3H), 3.94 (s, 3H).

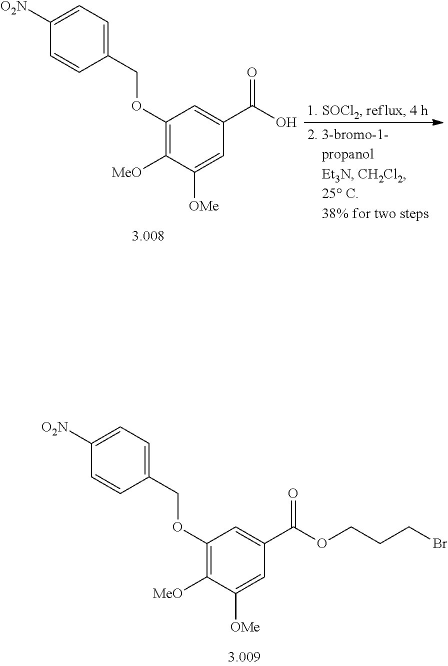 [SMILES: COc1cc(C(=O)O)cc(OCc2ccc([N+](=O)[O-])cc2)c1OC ; "1. SOCl2, reflux, 4 h" ; COc1cc(C(=O)OCCCBr)cc(OCc2ccc([N+](=O)[O-])cc2)c1OC]

To a solution of 3.008 (34 mg, 0.10 mmol) was added SOCl₂ (0.5 mL) at 25° C. The reaction mixture was then heated to reflux for 4 h. Upon cooling to 25° C., the reaction mixture was concentrated in vacuo. To the crude mixture in CH₂Cl₂ (0.25 mL) was added 3-bromo-1-propanol (0.01 mL, 0.11 mmol) and Et₃N (0.05 mL) at 25° C. After stirring 18 h, the reaction was quenched with the addition of H₂O and the resulting mixture was diluted with CH₂Cl₂. The layers were separated, and the aqueous layer was extracted with CH₂Cl₂. The combined organic layers were dried over anhydrous Na₂SO₄, filtered, and concentrated in vacuo. The residue was purified by column chromatography (silica gel, hexanes/EtOAc, 3/1) to afford 3.009 (17 mg, 38% for 2 steps). ¹H NMR (500 MHz, CDCl₃) δ 8.26 (d, J=8.8 Hz, 2H), 7.64 (d, J=8.8 Hz, 2H), 7.32 (d, J=1.8 Hz, 1H), 7.29 (d, J=1.8 Hz, 1H), 5.26 (s, 2H), 4.44 (t, J=6.1 Hz, 2H), 3.94 (s, 3H), 3.92 (s, 3H), 3.50 (t, J=6.5 Hz, 2H), 2.31 (dt, J=6.3, 6.3 Hz, 2H).

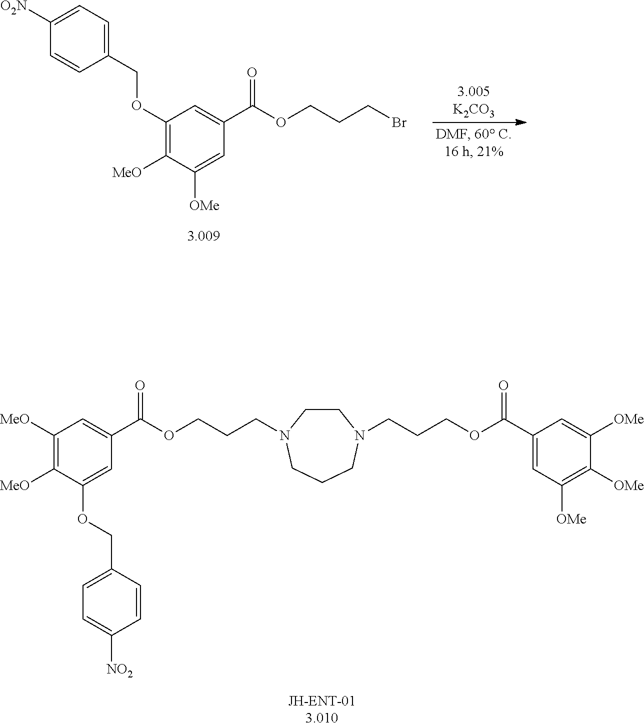 [SMILES: COc1cc(C(=O)OCCCBr)cc(OCc2ccc([N+](=O)[O-])cc2)c1OC ; "3.005K2CO3" ; COc1cc(C(=O)OCCCN2CCCN(CCCOC(=O)c3cc(OC)c(OC)c(OCc4ccc([N+](=O)[O-])cc4)c3)CC2)cc(OC)c1OC]

To a solution of 3.009 (17 mg, 0.04 mmol) and 3.005 (30 mg, 0.07 mmol) in DMF (0.14 mL) was added K₂CO₃ at 25° C. The reaction mixture was then heated to 60° C. for 16 h. Upon cooling to 25° C., the reaction was quenched with the addition of H₂O and the resulting mixture was diluted with EtOAc. The layers were separated, and the aqueous layer was extracted with EtOAc. The combined organic layers dried over anhydrous Na₂SO₄, filtered, and concentrated in vacuo. The residue was purified by column chromatography (silica gel, CH₂Cl₂/MeOH, 10/1) to afford 3.010 (6 mg, 21%). ¹H NMR (500 MHz, CD₃OD) δ 8.27-8.25 (m, 2H), 7.72 (s, 1H), 7.71 (s, 1H), 7.34 (s, 2H), 7.31 (s, 1H), 7.30 (s, 1H), 5.31 (s, 2H), 4.39-4.35 (m, 4H), 3.89 (s, 6H), 3.86 (s, 6H), 3.82 (s, 3H), 2.96-2.91 (m, 8H), 2.87-2.79 (m, 4H), 2.06-1.92 (m, 6H).

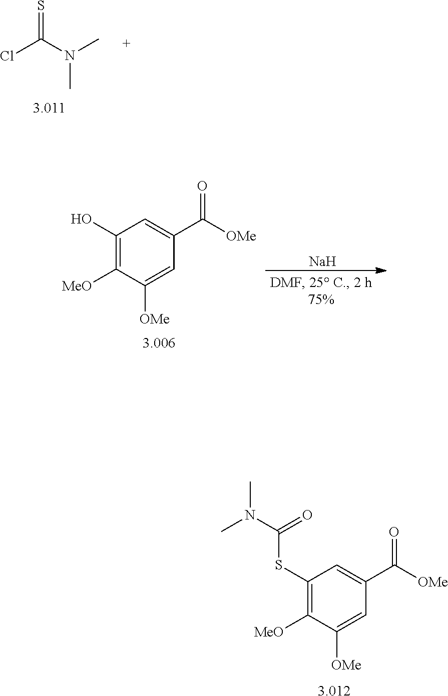

To a solution of NaH (60% in mineral oil) (112 mg, 2.84 mmol) in DMF (6 mL) was added 3,4-dimethoxy-5-hydroxybenzoic acid methyl ester (136 mg, 0.64 mmol) and dimethylthiocarbamoyl chloride (439 mg, 3.55 mmol) at 25° C. After stirring for 2 h, the reaction was diluted with EtOAc and filtered through celite. The filtrate was then washed with H₂O and the organic layer dried over anhydrous Na₂SO₄, filtered, and concentrated in vacuo. The residue was purified by column chromatography (silica gel, hexanes/EtOAc, 20/1 to 5/1) to afford 3.012 as a white solid (144 mg, 75%). ¹H NMR (500 MHz, CDCl₃) δ 7.52 (d, J=1.8 Hz, 1H), 7.40 (d, J=1.9 Hz, 1H), 3.94 (s, 3H), 3.93 (s, 3H), 3.88 (s, 3H), 3.46 (s, 3H), 3.37 (s, 3H).

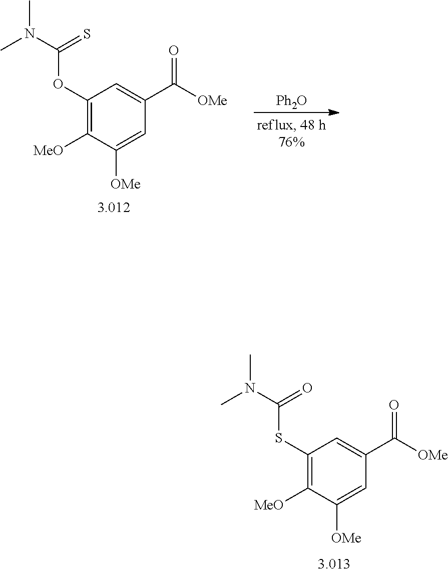

A solution of 3.012 (144 mg, 0.48 mmol) in Ph₂O (0.72 mL) was warmed to reflux for 48 h. After cooling to 25° C., the crude material was purified by column chromatography (silica gel, hexanes/EtOAc, 20/1 to 5/1) to afford 3.013 (110 mg, 76%). ¹H NMR (500 MHz, CDCl₃) δ 7.83 (br s, 1H), 7.66 (br s, 1H), 3.93 (s, 6H), 3.90 (s, 3H), 3.15 (br s, 3H), 3.04 (br s, 3H).

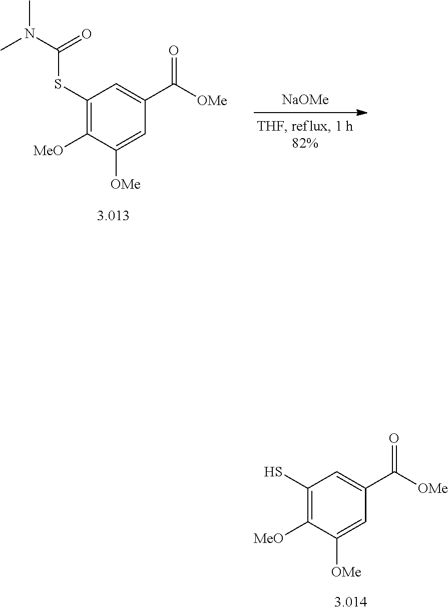 [SMILES: COC(=O)c1cc(OC)c(OC)c(SC(=O)N(C)C)c1 ; COC(=O)c1cc(S)c(OC)c(OC)c1 ; "NaOMe"]

To a solution of 3.013 (203 mg, 0.68 mmol) in THE (6.0 mL) was added MeONa (0.5 M in methanol, 0.68 mmol) at 25° C. The reaction mixture was then heated to reflux for 1 h. Upon cooling to 25° C., the reaction mixture was quenched with 1 M HCl and diluted with EtOAc. The layers were separated, and the aqueous layer was extracted with EtOAc. The combined organic layers were washed with brine and dried over anhydrous Na₂SO₄, filtered and concentrated in vacuo. The residue was purified by column chromatography (silica gel, hexanes/EtOAc, 5/1) to afford 3.014 (127 mg, 82%). ¹H NMR (400 MHz, CDCl₃) δ 7.57 (d, J=1.7 Hz, 1H), 7.35 (d, J=1.7 Hz, 1H), 3.90 (s, 3H), 3.89 (s, 3H), 3.88 (s, 3H).

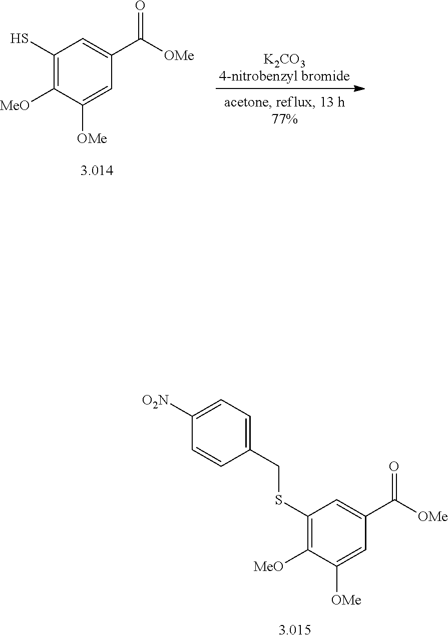 [SMILES: COC(=O)c1cc(S)c(OC)c(OC)c1 ; "K2CO34-nitrobenzyl bromide" ; COC(=O)c1cc(OC)c(OC)c(SCc2ccc([N+](=O)[O-])cc2)c1]

To a solution of 3.014 (127 mg, 0.56 mmol) in acetone (2.2 mL) was added 4-nitrobenzyl bromide (179 mg, 0.83 mmol) and K₂CO₃ (123 mg, 0.89 mmol) at 25° C. The reaction mixture was then heated to reflux for 20 h. Upon cooling to 0° C., the reaction was quenched with an addition of saturated aqueous NH₄Cl and the resulting mixture was diluted with EtOAc. The layers were separated, and the aqueous layer was extracted with EtOAc. The combined organic layers were dried over anhydrous Na₂SO₄, filtered, and concentrated in vacuo. The residue was purified by column chromatography (silica gel, hexanes/EtOAc, 5/1) to afford 3.015 (157 mg, 77%). ¹H NMR (400 MHz, CDCl₃) δ 8.12 (d, J=8.3 Hz, 2H), 7.50 (br s, 1H), 7.47 (d, J=8.4 Hz, 2H), 7.45 (br s, 1H), 4.20 (s, 2H), 3.90 (s, 6H), 3.87 (s, 3H).

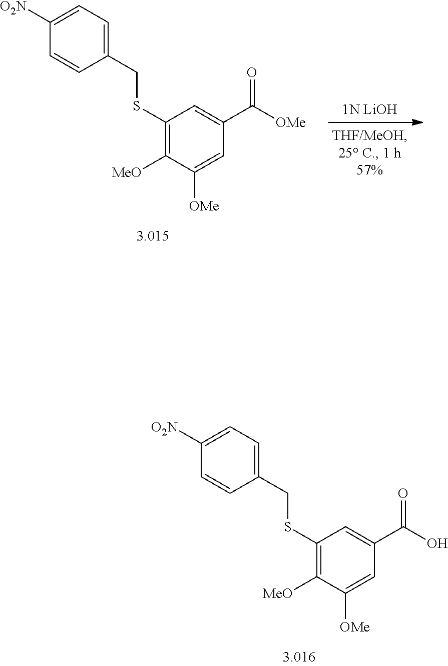 [SMILES: COC(=O)c1cc(OC)c(OC)c(SCc2ccc([N+](=O)[O-])cc2)c1 ; "1N LiOH" ; COc1cc(C(=O)O)cc(SCc2ccc([N+](=O)[O-])cc2)c1OC]

To a solution of 3.015 (157 mg, 0.43 mmol), in THF/MeOH (3/1) (14 mL) was added 1 N LiOH (2.6 mL) at 25° C. After stirring for 1 h, 1 M HCl was added until the solution became acidic and the resulting mixture was diluted with CH₂Cl₂. The layers were separated, and the aqueous layer was extracted with CH₂Cl₂. The combined organic layers were dried over anhydrous Na₂SO₄, filtered, and concentrated in vacuo. The residue was purified by column chromatography (silica gel, CH₂Cl₂/MeOH, 10/1) to afford 3.016 (80 mg, 53%). ¹H NMR (400 MHz, CDCl₃) δ 8.11 (d, J=8.4 Hz, 2H), 7.57 (s, 1H), 7.50 (s, 1H), 7.47 (d, J=8.2 Hz, 2H), 4.19 (s, 2H), 3.90 (s, 3H), 3.89 (s, 3H).

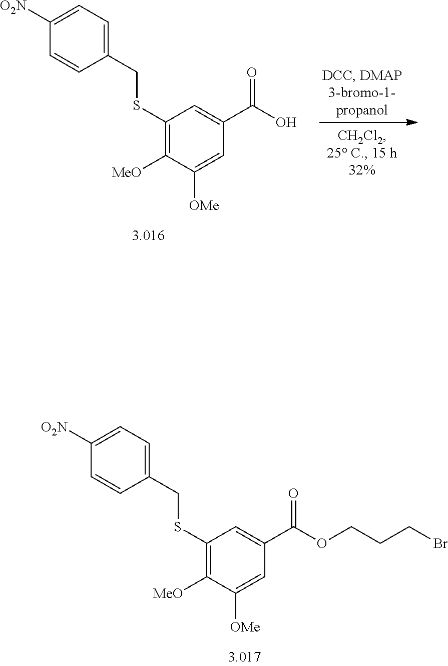 [SMILES: COc1cc(C(=O)O)cc(SCc2ccc([N+](=O)[O-])cc2)c1OC ; "DCC, DMAP3-bromo-1-propanol" ; COc1cc(C(=O)OCCCBr)cc(SCc2ccc([N+](=O)[O-])cc2)c1OC]

To a solution of 3.016 (7 mg, 0.02 mmol) in CH₂Cl₂ (0.4 mL) was added 3-bromo-1-propanol (3 mg, 0.022 mmol), DCC (5 mg, 0.024 mmol), and DMAP (0.002 mmol) at 25° C. After stirring for 15 h, the reaction was concentrated in vacuo. The residue was purified by column chromatography (silica gel, hexanes/EtOAc, 5/1) to afford 3.017 (3 mg, 32%). ¹H NMR (500 MHz, CDCl₃) δ 8.13 (d, J=8.7 Hz, 2H), 7.49 (d, J=8.7 Hz, 2H), 7.47 (d, J=1.9 Hz, 1H), 7.45 (d, J=1.8 Hz, 1H), 4.43 (t, J=6.1 Hz, 2H), 4.21 (s, 2H), 3.91 (s, 3H), 3.90 (s, 3H), 3.49 (t, J=6.5 Hz, 2H), 2.29 (dt, J=6.3, 6.3 Hz, 2H).

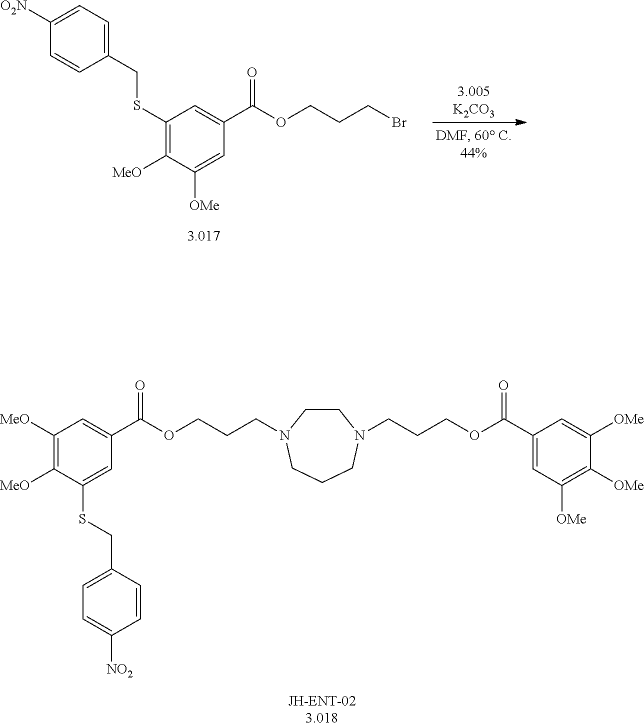 [SMILES: COc1cc(C(=O)OCCCBr)cc(SCc2ccc([N+](=O)[O-])cc2)c1OC ; "3.005K2CO3" ; COc1cc(C(=O)OCCCN2CCCN(CCCOC(=O)c3cc(OC)c(OC)c(SCc4ccc([N+](=O)[O-])cc4)c3)CC2)cc(OC)c1OC]

To a solution of 3.017 (3 mg, 0.006 mmol) and 3.005 (4 mg, 0.012 mmol) in DMF (0.05 mL) was added K₂CO₃ (8 mg, 0.06 mmol) at 25° C. The reaction was then heated to 60° C. for 3 days. Upon cooling to 25° C., the reaction was quenched with the addition of H₂O and the resulting mixture was diluted with EtOAc. The layers were separated, and the aqueous layer was extracted with EtOAc. The combined organic layers dried over anhydrous Na₂SO₄, filtered, and concentrated in vacuo. The residue was purified by column chromatography (silica gel, CH₂Cl₂/MeOH, 10/1) to afford 3.018 (2 mg, 44%). ¹H NMR (500 MHz, CD₃OD) δ 8.13 (d, J=8.3 Hz, 2H), 7.56 (d, J=8.2 Hz, 2H), 7.47 (s, 1H), 7.44 (s, 1H), 7.31 (s, 2H), 4.36 (t, J=6.3 Hz, 2H), 4.31 (t, J=6.3 Hz, 2H), 3.88 (s, 3H), 3.87 (s, 9H), 3.82 (s, 3H), 2.80-2.75 (m, 8H), 2.69 (dd, J=7.3, 7.4 Hz, 2H), 2.63 (dd, J=7.3, 7.2 Hz, 2H), 2.00-1.90 (m, 4H), 1.84 (dt, J=10.1, 5.3 Hz, 2H).

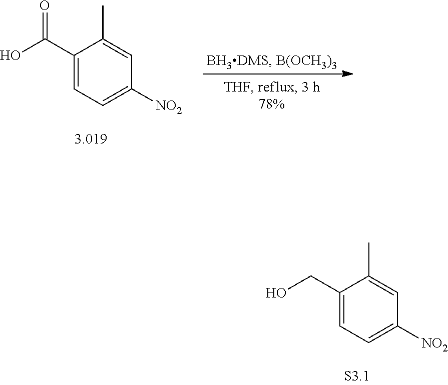

To a solution of 2-methyl-4-nitrobenzoic acid (200 mg, 1.1 mmol) in THF (11 mL) was added BH₃·DMS (0.2 mL, 3.3 mmol) and B(OCH₃)₃ (0.48 mL, 5.0 mmol) at 25° C. The reaction was then warmed to reflux for 3 h. Upon cooling to 25° C., the reaction was quenched with an addition of methanol and 1 M HCl the resulting mixture was diluted with EtOAc and H₂O. The layers were separated, and the aqueous layer was extracted with EtOAc. The combined organic layers were washed with brine and dried over anhydrous Na₂SO₄, filtered and concentrated in vacuo. The residue was purified by column chromatography (silica gel, hexanes/EtOAc, 2/1) to afford S3.1 (143 mg, 78%). ¹H NMR (500 MHz, CDCl₃) δ 8.08 (dd, J=8.4, 2.2 Hz, 1H), 8.03 (d, J=2.0 Hz, 1H), 7.62 (d, J=8.4 Hz, 1H), 4.80 (s, 2H), 2.40 (s, 3H).

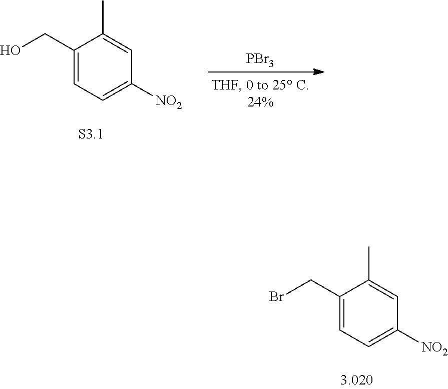

To a cooled (0° C.) solution of S3.1 (70 mg, 0.42 mmol) in CH₂Cl₂ (8.4 mL) was added PBr₃ (0.04 mL, 0.84 mmol). The reaction was then allowed to slowly warm to 25° C. After stirring for 4 h, the reaction was quenched with an addition of H₂O. The layers were separated, and the aqueous layer was extracted with CH₂Cl₂. The combined organic layers were dried over anhydrous Na₂SO₄, filtered, and concentrated in vacuo. The residue was purified by column chromatography (silica gel, hexanes/EtOAc, 10/1) to afford 3.020 (23 mg, 24%). ¹H NMR (500 MHz, CDCl₃) δ 8.06 (d, J=2.0 Hz, 1H), 8.02 (dd, J=8.4, 2.3 Hz, 1H), 7.47 (d, J=8.4 Hz, 1H), 4.51 (s, 2H), 2.51 (s, 3H).

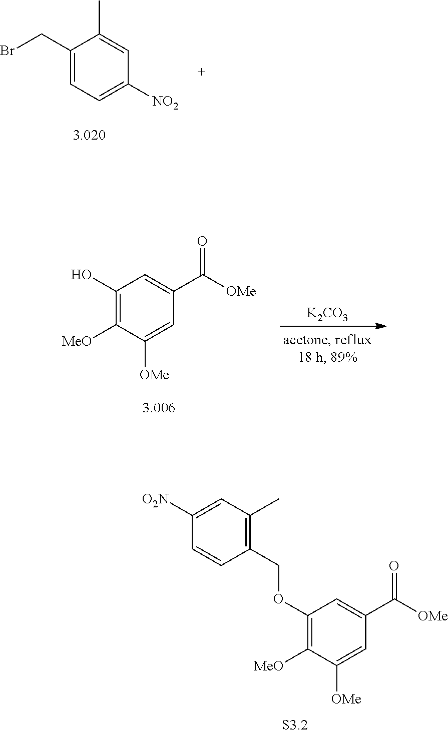

To a solution of 3.020 (94 mg, 0.41 mmol) in acetone (1 mL) was added 3,4-dimethoxy-5-hydroxybenzoic acid methyl ester (58 mg, 0.27 mmol) and K₂CO₃ (59 mg, 0.43 mmol) at 25° C. The reaction mixture was then heated to reflux for 18 h. Upon cooling to 0° C., the reaction was quenched with an addition of saturated aqueous NH₄Cl and the resulting mixture was diluted with EtOAc. The layers were separated, and the aqueous layer was extracted with EtOAc. The combined organic layers were dried over anhydrous Na₂SO₄, filtered, and concentrated in vacuo. The residue was purified by column chromatography (silica gel, hexanes/EtOAc, 5/1) to afford S3.2 (88 mg, 89%). ¹H NMR (400 MHz, CDCl₃) δ 8.10-8.06 (m, 2H), 7.68 (d, J=9.0 Hz, 1H), 7.34 (s, 2H), 5.18 (s, 2H), 3.92 (s, 3H), 3.91 (s, 3H), 3.90 (s, 3H), 2.48 (s, 3H).

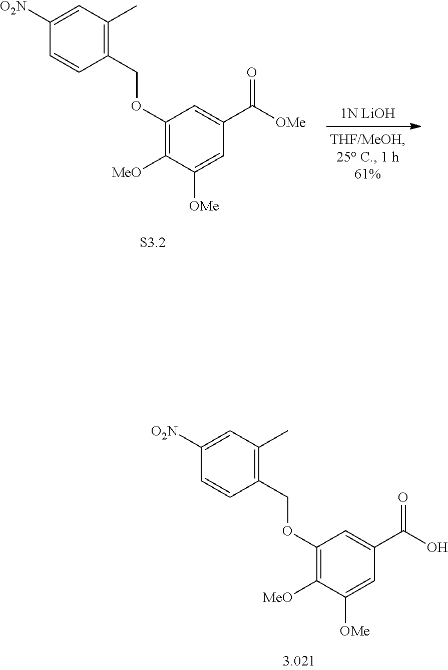 [SMILES: COC(=O)c1cc(OC)c(OC)c(OCc2ccc([N+](=O)[O-])cc2C)c1 ; "1N LiOH" ; COc1cc(C(=O)O)cc(OCc2ccc([N+](=O)[O-])cc2C)c1OC]

To a solution of S3.2 (53 mg, 0.15 mmol), in THF/MeOH (3/1) (5 mL) was added 1 N LiOH (0.88 mL) at 25° C. After stirring for 1 h, 1 M HCl was added until the solution became acidic, and the resulting mixture was diluted with CH₂Cl₂. The layers were separated, and the aqueous layer was extracted with CH₂Cl₂. The combined organic layers were dried over anhydrous Na₂SO₄, filtered, and concentrated in vacuo. The residue was purified by column chromatography (silica gel, CH₂Cl₂/MeOH, 10/1) to afford 3.021 (31 mg, 61%). ¹H NMR (500 MHz, CDCl₃) δ 8.11-8.06 (m, 2H), 7.68 (d, J=9.0 Hz, 1H), 7.41 (s, 1H), 7.40 (s, 1H), 5.20 (s, 2H), 3.94 (s, 6H), 2.49 (s, 3H).

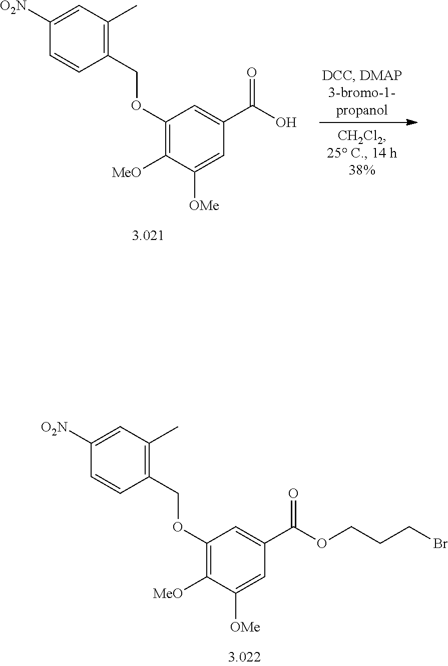 [SMILES: COc1cc(C(=O)O)cc(OCc2ccc([N+](=O)[O-])cc2C)c1OC ; "DCC, DMAP3-bromo-1-propanol" ; COc1cc(C(=O)OCCCBr)cc(OCc2ccc([N+](=O)[O-])cc2C)c1OC]

To a solution of 3.021 (31 mg, 0.09 mmol) in CH₂Cl₂ (2 mL) was added 3-bromo-1-propanol (14 mg, 0.98 mmol), DCC (23 mg, 0.11 mmol), and DMAP (0.009 mmol) at 25° C. After stirring for 14 h, the reaction was concentrated in vacuo. The residue was purified by column chromatography (silica gel, hexanes/EtOAc, 5/1) to afford 3.022 (16 mg, 38%). ¹H NMR (500 MHz, CDCl₃) δ 8.10-8.06 (m, 2H), 7.67 (d, J=9.1 Hz, 1H), 7.33 (s, 1H), 7.32 (s, 1H), 5.19 (s, 2H), 4.46 (t, J=6.1 Hz, 2H), 3.92 (s, 3H), 3.91 (s, 3H), 3.51 (t, J=6.5 Hz, 2H), 2.49 (s, 3H), 2.32 (dt, J=6.3, 6.3 Hz, 2H).

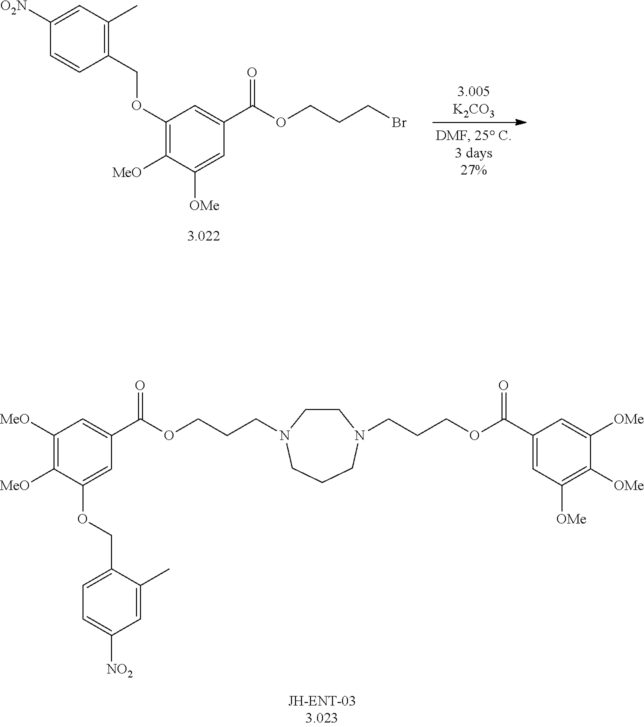 [SMILES: COc1cc(C(=O)OCCCBr)cc(OCc2ccc([N+](=O)[O-])cc2C)c1OC ; "3.005K2CO3" ; COc1cc(C(=O)OCCCN2CCCN(CCCOC(=O)c3cc(OC)c(OC)c(OCc4ccc([N+](=O)[O-])cc4C)c3)CC2)cc(OC)c1OC]

To a solution of 3.022 (8 mg, 0.017 mmol) and 3.005 (11 mg, 0.03 mmol) in DMF (0.14 mL) was added K₂CO₃ (24 mg, 0.17 mmol) at 25° C. After stirring for 3 days, the reaction was quenched with the addition of H₂O and the resulting mixture was diluted with EtOAc. The layers were separated, and the aqueous layer was extracted with EtOAc. The combined organic layers dried over anhydrous Na₂SO₄, filtered, and concentrated in vacuo. The residue was purified by column chromatography (silica gel, CH₂Cl₂/MeOH, 10/1) to afford 3.023 (3.4 mg, 27%). ¹H NMR (500 MHz, CD₃OD) δ 8.11 (br s, 1H), 8.07 (d, J=8.5 Hz, 1H), 7.70 (d, J=8.5 Hz, 1H), 7.36 (br s, 2H), 7.31 (br s, 2H), 5.27 (s, 2H), 4.41-4.36 (m, 4H), 3.89 (s, 3H), 3.86 (s, 9H), 3.82 (s, 3H), 3.03-2.93 (m, 8H), 2.90-2.83 (m, 4H), 2.51 (s, 3H), 2.07-2.01 (m, 4H), 1.99-1.93 (m, 2H).

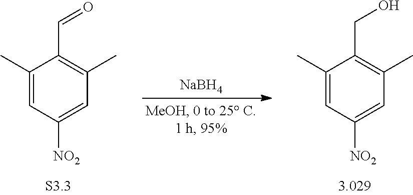

Compound S3.3 was prepared according to the previously reported procedure. 21 To a cooled solution (0° C.) of S3.3 (373 mg, 2.08 mmol) in MeOH (40 mL) was added NaBH₄ (157 mg, 4.16 mmol). After slowly warming to 25° C. for 1 h, the reaction was quenched with an addition of saturated aqueous NaHCO₃, and the resulting mixture was diluted with EtOAc. The layers were separated, and the aqueous layer was extracted with EtOAc. The combined organic layers were dried over anhydrous Na₂SO₄, filtered, and concentrated in vacuo. The residue was purified by column chromatography (silica gel, hexanes/EtOAc, 2/1) to afford 3.029 (355 mg, 95%). ¹H NMR (400 MHz, CDCl₃) δ 7.90 (s, 2H), 4.79 (s, 2H), 2.53 (s, 6H).

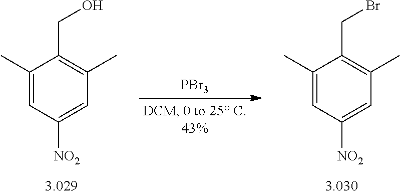

To a cooled (0° C.) solution of 3.029 (275 mg, 1.52 mmol) in CH₂Cl₂ (3 mL) was added PBr3 (0.36 mL, 3.8 mmol). The reaction was then allowed to slowly warm to 25° C. After stirring for 4 h, the reaction was quenched with an addition of H₂O. The layers were separated, and the aqueous layer was extracted with CH₂Cl₂. The combined organic layers were dried over anhydrous Na₂SO₄, filtered, and concentrated in vacuo. The residue was purified by column chromatography (silica gel, hexanes/EtOAc, 10/1) to afford 3.030 (160 mg, 43%). ¹H NMR (500 MHz, CDCl₃) δ 7.90 (s, 2H), 4.52 (s, 2H), 2.51 (s, 6H).

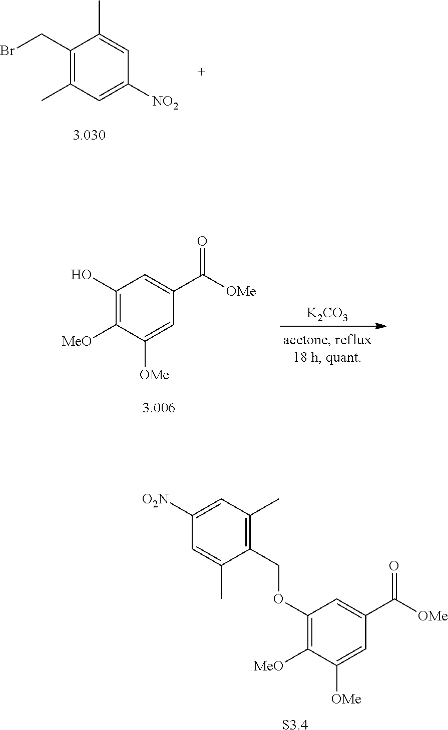

To a solution of 3.030 (150 mg, 0.61 mmol) in acetone (2 mL) was added 3,4-dimethoxy-5-hydroxybenzoic acid methyl ester (155 mg, 0.73 mmol) and K₂CO₃ (126 mg, 0.92 mmol) at 25° C. The reaction mixture was then heated to reflux for 18 h. Upon cooling to 0° C., the reaction was quenched with an addition of saturated aqueous NH₄Cl, and the resulting mixture was diluted with EtOAc. The layers were separated, and the aqueous layer was extracted with EtOAc. The combined organic layers were dried over anhydrous Na₂SO₄, filtered, and concentrated in vacuo. The residue was purified by column chromatography (silica gel, hexanes/EtOAc, 5/1) to afford S3.4 (229 mg, quantitative). ¹H NMR (500 MHz, CDCl₃) δ 7.93 (s, 2H), 7.44 (d, J=1.7 Hz, 1H), 7.36 (d, J=1.6 Hz, 1H), 5.14 (s, 2H), 3.93 (s, 3H), 3.92 (s, 3H), 3.82 (s, 3H), 2.53 (s, 6H).

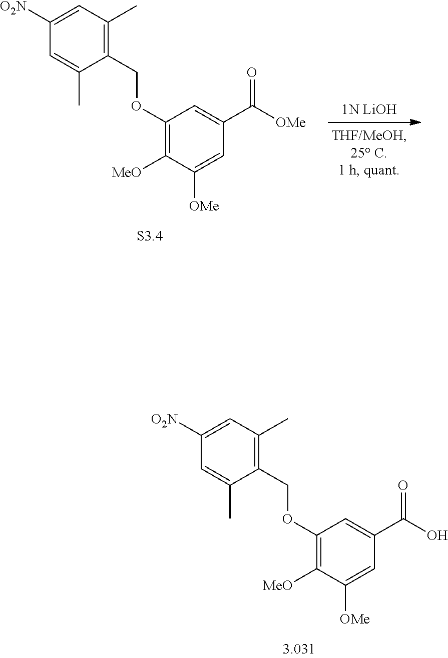 [SMILES: COC(=O)c1cc(OC)c(OC)c(OCc2c(C)cc([N+](=O)[O-])cc2C)c1 ; "1N LiOH" ; COc1cc(C(=O)O)cc(OCc2c(C)cc([N+](=O)[O-])cc2C)c1OC]

To a solution of S3.4 (200 mg, 0.53 mmol), in THF/MeOH (3/1) (18 mL) was added 1 N LiOH (3.2 mL) at 25° C. After stirring for 1 h, 1 M HCl was added until the solution became acidic, and the resulting mixture was diluted with CH₂Cl₂. The layers were separated, and the aqueous layer was extracted with CH₂Cl₂. The combined organic layers were dried over anhydrous Na₂SO₄, filtered, and concentrated in vacuo. The residue was purified by column chromatography (silica gel, CH₂Cl₂/MeOH, 10/1) to afford 3.031 (192 mg, quantitative). ¹H NMR (500 MHz, CDCl₃) δ 7.94 (s, 2H), 7.51 (d, J=1.6 Hz, 1H), 7.43 (d, J=1.6 Hz, 1H), 5.16 (s, 2H), 3.94 (s, 3H), 3.85 (s, 3H), 2.54 (s, 6H).

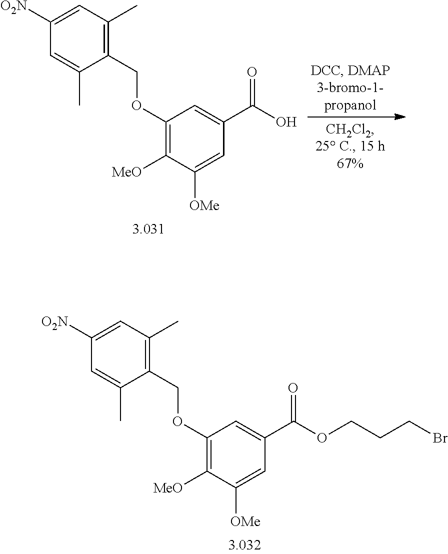 [SMILES: COc1cc(C(=O)O)cc(OCc2c(C)cc([N+](=O)[O-])cc2C)c1OC ; "DCC, DMAP3-bromo-1-propanol" ; COc1cc(C(=O)OCCCBr)cc(OCc2c(C)cc([N+](=O)[O-])cc2C)c1OC]

To a solution of 3.031 (155 mg, 0.43 mmol) in CH₂Cl₂ (9 mL) was added 3-bromo-1-propanol (65 mg, 0.47 mmol), DCC (107 mg, 0.52 mmol), and DMAP (0.043 mmol) at 25° C. After stirring for 15 h, the reaction was concentrated in vacuo. The residue was purified by column chromatography (silica gel, hexanes/EtOAc, 5/1) to afford 3.032 (140 mg, 67%). ¹H NMR (500 MHz, CDCl₃) δ 7.94 (s, 2H), 7.42 (d, J=1.6 Hz, 1H), 7.35 (d, J=1.6 Hz, 1H), 5.14 (s, 2H), 4.49 (t, J=6.1 Hz, 2H), 3.92 (s, 3H), 3.83 (s, 3H), 3.54 (t, J=6.5 Hz, 2H), 2.53 (s, 6H), 2.35 (dt, J=6.3, 6.3 Hz, 2H).

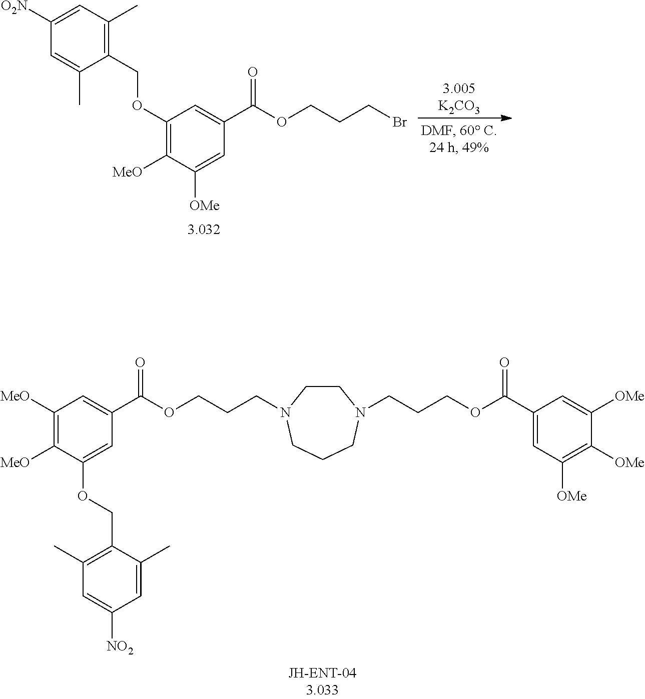 [SMILES: COc1cc(C(=O)OCCCBr)cc(OCc2c(C)cc([N+](=O)[O-])cc2C)c1OC ; "3.005K2CO3" ; COc1cc(C(=O)OCCCN2CCCN(CCCOC(=O)c3cc(OC)c(OC)c(OCc4c(C)cc([N+](=O)[O-])cc4C)c3)CC2)cc(OC)c1OC]

To a solution of 3.032 (45 mg, 0.09 mmol) and 3.005 (63 mg, 0.18 mmol) in DMF (0.75 mL) was added K₂CO₃ (124 mg, 0.9 mmol) at 25° C. After stirring for 3 days, the reaction was quenched with the addition of H₂O and the resulting mixture was diluted with EtOAc. The layers were separated, and the aqueous layer was extracted with EtOAc. The combined organic layers dried over anhydrous Na₂SO₄, filtered, and concentrated in vacuo. The residue was purified by column chromatography (silica gel, CH₂Cl₂/MeOH, 10/1) to afford 3.033 (33 mg, 49%). ¹H NMR (500 MHz, CDCl₃) δ 7.92 (s, 2H), 7.41 (d, J=1.8 Hz, 1H), 7.33 (d, J=1.8 Hz, 1H), 7.27 (s, 2H), 5.14 (s, 2H), 4.39-4.35 (m, 4H), 3.91 (s, 3H), 3.90 (s, 9H), 3.82 (s, 3H), 2.95-2.83 (m, 8H), 2.78-2.74 (m, 4H), 2.52 (s, 6H), 2.05-1.92 (m, 6H).

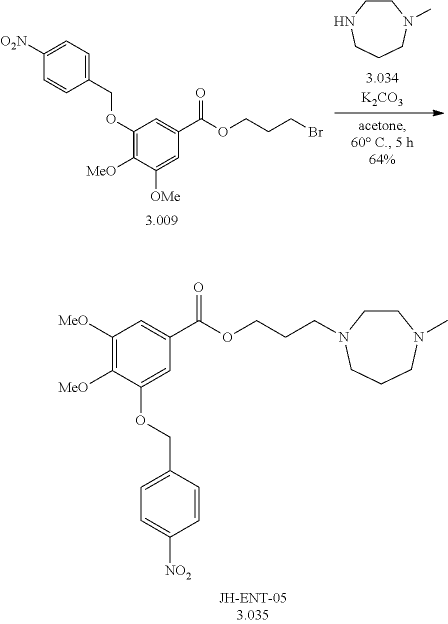

To a solution of 3.009 (34 mg, 0.08 mmol) in acetone (0.25 mL) was added 1-methylhomopiperazine (17 mg, 0.15 mmol) and K₂CO₃ (104 mg, 0.75 mmol) at 25° C. The reaction mixture was then warmed to 60° C. for 5 h. Upon cooling to 25° C., the reaction was quenched with the addition of saturated aqueous NH₄Cl, and the resulting mixture was diluted with CH₂Cl₂. The layers were separated, and the aqueous layer was extracted with CH₂Cl₂. The combined organic layers dried over anhydrous Na₂SO₄, filtered, and concentrated in vacuo. The residue was purified by column chromatography (silica gel, CH₂Cl₂/MeOH, 10/1) to afford 3.035 (23 mg, 64%). ¹H NMR (500 MHz, CDCl₃) δ 8.25 (d, J=8.8 Hz, 2H), 7.63 (d, J=8.8 Hz, 2H), 7.32 (d, J=1.8 Hz, 1H), 7.29 (d, J=1.8 Hz, 1H), 5.26 (s, 2H), 4.34 (t, J=6.6 Hz, 2H), 3.93 (s, 3H), 3.91 (s, 3H), 2.73-2.70 (m, 4H), 2.63-2.59 (m, 6H), 2.35 (s, 3H), 1.91 (dt, J=13.7, 6.7 Hz, 2H), 1.81 (dt, J=5.9, 5.9 Hz, 2H).

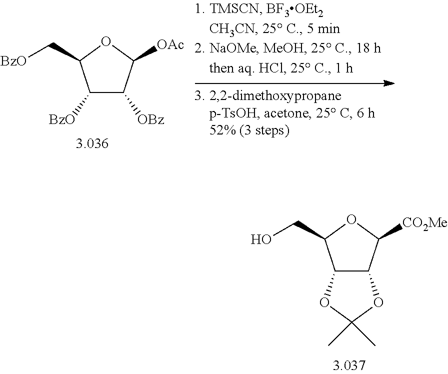

To a solution of β-D-ribofuranose-1-acetate 2,3,5-tribenzoate (10.1 g, 20.0 mmol) in CH₃CN (120 mL) was added TMSCN (10 mg, 80.0 mmol) and a few drops of BF₃·OEt₂ at 25° C. After stirring for 5 min, the reaction was quenched with an addition of saturated aqueous NaHCO₃ and diluted with Et₂O. The layers were separated, and the aqueous layer was extracted with Et₂O. The combined organic layers were dried over anhydrous Na₂SO₄, filtered, and concentrated in vacuo. The crude residue was dissolved in MeOH (200 mL) and NaOMe (0.5 M in MeOH, 40 mL) was added at 25° C. After stirring for 18 h, 1 M HCl was added until the solution became acidic. After stirring 1 h, the reaction was concentrated in vacuo. The crude residue was dissolved in acetone (100 mL) and 2,2-dimethoxypropane (7.4 mL, 60.0 mmol) and p-TsOH (1.9 g, 10.0 mmol) were added at 25° C. After stirring for 6 h, the reaction was quenched with the addition of H2O and the resulting mixture was diluted with EtOAc. The layers were separated, and the aqueous layer was extracted with EtOAc. The combined organic layers dried over anhydrous Na2SO4, filtered, and concentrated in vacuo. The residue was purified by column chromatography (silica gel, hexane/EtOAc, 2/3) to afford 3.037 (2.4 g, 52% for 3 steps). ¹H NMR (400 MHz, CDCl₃) δ 4.85 (dd, J=6.0, 3.1 Hz, 1H), 4.75 (dd, J=6.1, 1.7 Hz, 1H), 4.59 (d, J=3.1 Hz, 1H), 4.40 (br s, 1H), 3.85 (dd, J=12.5, 2.8 Hz, 1H), 3.81 (s, 3H), 3.54 (dd, J=12.5, 3.7 Hz, 1H), 1.55 (s, 3H), 1.35 (s, 3H).

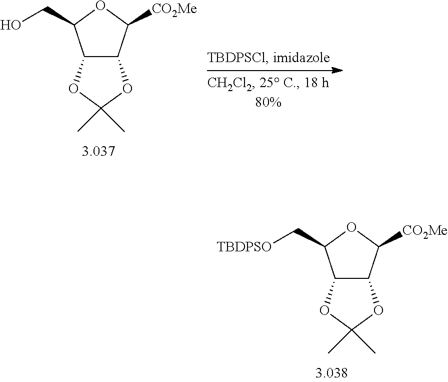

To a solution of 3.037 (1.21 g, 5.2 mmol) in CH₂Cl₂ (60 mL) was added TBDPSCl (2.57 mg, 10.4 mmol) and imidazole (1.07 g, 15.6 mmol) at 25° C. After stirring for 18 h, the reaction was quenched with the addition of H2O and the resulting mixture was diluted with CH₂Cl₂. The layers were separated, and the aqueous layer was extracted with CH₂Cl₂. The combined organic layers dried over anhydrous Na₂SO₄, filtered, and concentrated in vacuo. The residue was purified by column chromatography (silica gel, hexane/EtOAc, 13/1) to afford 3.038 (1.95 g, 80%). ¹H NMR (400 MHz, CDCl₃) δ 7.67-7.63 (m, 4H), 7.44-7.35 (m, 6H), 4.91 (dd, J=6.2, 3.8 Hz, 1H), 4.73 (dd, J=6.2, 2.0 Hz, 1H), 4.49 (d, J=3.9 Hz, 1H), 4.26 (td, J=4.4, 2.1 Hz, 1H), 3.73 (d, J=4.4 Hz, 2H), 3.68 (s, 3H), 1.54 (s, 3H), 1.35 (s, 3H), 1.03 (s, 3H).

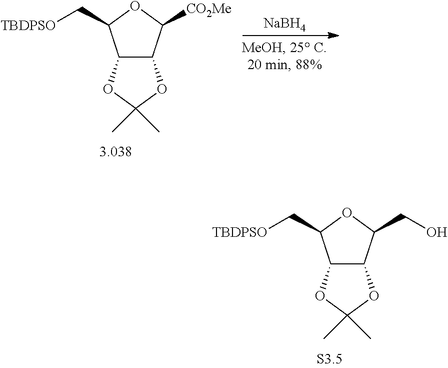

To a solution of 3.038 (1.95 g, 4.1 mmol) in MeOH (45 mL) was added NaBH₄ (784 mg, 20.7 mmol) at 25° C. After stirring for 20 min, the reaction was quenched with the addition of a saturated aqueous solution of NH₄Cl, and the resulting mixture was diluted with EtOAc. The layers were separated, and the aqueous layer was extracted with EtOAc. The combined organic layers dried over anhydrous Na₂SO₄, filtered, and concentrated in vacuo. The residue was purified by column chromatography (silica gel, hexane/EtOAc, 4/1) to afford S3.5 (1.62 g, 88%). ¹H NMR (500 MHz, CDCl₃) δ 7.69-7.66 (m, 4H), 7.47-7.37 (m, 6H), 4.79 (dd, J=6.4, 4.5 Hz, 1H), 4.70 (dd, J=6.4, 3.3 Hz, 1H), 4.17 (ddd, J=3.2, 3.2, 3.2 Hz, 1H), 4.05 (ddd, J=4.4, 3.0, 3.0 Hz, 1H), 3.90 (dd, J=11.4, 3.1 Hz, 1H), 3.84 (dd, J=11.9, 2.9, Hz, 1H), 3.75 (dd, 11.4, 3.0 Hz, 1H), 3.65 (dd, J=11.9, 3.4 Hz, 1H), 1.53 (s, 3H), 1.36 (s, 3H), 1.07 (s, 9H).

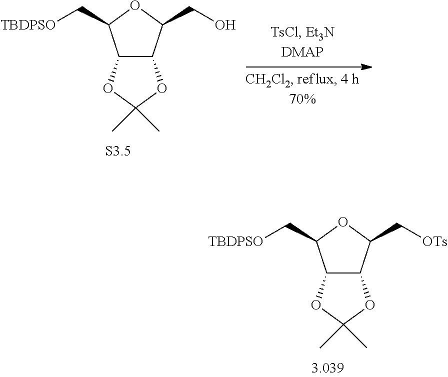

To a solution of S3.5 (670 mg, 1.52 mmol) in CH₂Cl₂ (8 mL) was added TsCl (317 mg, 1.66 mmol), Et₃N (0.25 mL, 1.82 mmol), and DMAP (18 mg, 0.15 mmol) at 25° C. After warming to reflux, the reaction was stirred for 4 h. After cooling to 25° C., the reaction was diluted with CH₂Cl₂, and washed with 0.5 M HCl. The combined organic layers dried over anhydrous Na₂SO₄, filtered, and concentrated in vacuo. The residue was purified by column chromatography (silica gel, hexane/EtOAc, 9/2) to afford 3.039 (640 mg, 70%). ¹H NMR (500 MHz, CDCl₃) δ 7.73 (d, J=8.3 Hz, 2H), 7.65-7.64 (m, 4H), 4.66 (dd, J=6.5, 3.4 Hz, 1H), 4.5 (dd, J=6.4, 3.9 Hz, 1H), 4.12-4.09 (m, 3H), 4.07 (dt J=7.5, 3.6 Hz, 1H), 3.72-3.65 (m, 2H), 2.40 (s, 3H), 1.50 (s, 3H), 1.32 (s, 3H).

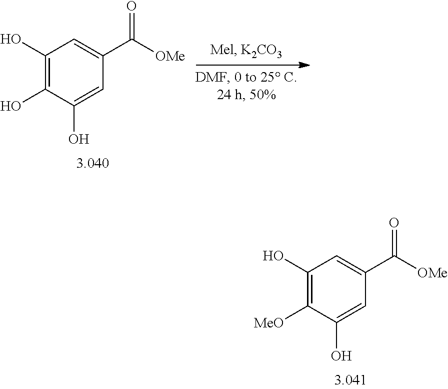 [SMILES: COC(=O)c1cc(O)c(O)c(O)c1 ; COC(=O)c1cc(O)c(OC)c(O)c1]

To a solution of methyl gallate (2.0 g, 16.3 mmol) in DMF (20 mL) was added K₂CO₃ (1.8 g, 19.5 mmol) at 25° C. After stirring for 1 h, the solution was cooled to 0° C. and Mel (1.05 mL, 16.9 mmol) was added. After stirring 30 min, the reaction was warmed to 25° C. After 24 h, the reaction was filtered and diluted with brine and EtOAc. The layers were separated, and the aqueous layer was extracted with EtOAc. The combined organic layers dried over anhydrous Na₂SO₄, filtered, and concentrated in vacuo. The residue was purified by column chromatography (silica gel, CHCl₃/MeOH 50/1) to afford 3.041 (1.6 g, 50%). ¹H NMR (500 MHz, CDCl₃) δ 9.54 (s, 2H), 7.00 (s, 2H), 3.83 (s, 3H), 3.79 (s, 3H).

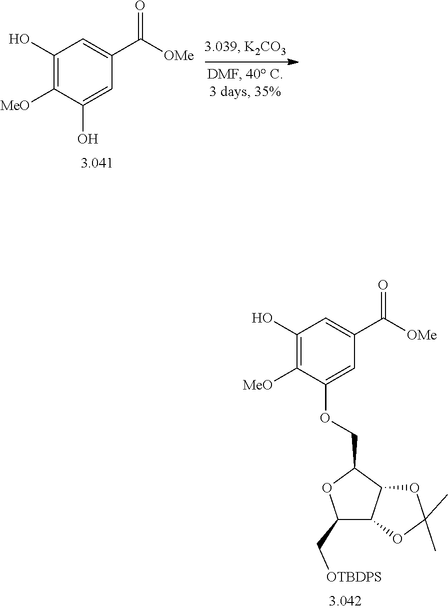

To a solution of 3.041 (425 mg, 2.14 mmol) in DMF (12 mL) was added K₂CO₃ (296 g, 2.14 mmol) and 3.039 (640 mg, 1.07 mmol) at 25° C. The reaction mixture was then heated to 40° C. for 3 days. Upon cooling to 0° C., the reaction was quenched with an addition of saturated aqueous NH₄Cl, and the resulting mixture was diluted with EtOAc. The layers were separated, and the aqueous layer was extracted with EtOAc. The combined organic layers were dried over anhydrous Na₂SO₄, filtered, and concentrated in vacuo. The residue was purified by column chromatography (silica gel, hexanes/EtOAc, 4/1) to afford 3.042 (466 mg, 35%). ¹H NMR (400 MHz, CDCl₃) δ 7.66-7.64 (m, 4H), 7.42-7.30 (m, 7H), 7.14 (d, J=1.7 Hz, 1H), 4.73 (dd, J=6.4, 3.3 Hz, 1H), 4.65-4.63 (m, 1H), 4.35 (ddd, J=4.9, 4.9, 4.8 Hz, 1H), 4.20-4.10 (m, 3H), 3.92 (s, 3H), 3.85 (s, 3H), 3.78 (t, J=4.0 Hz, 2H), 1.56 (s, 3H), 1.37 (s, 3H), 1.05 (s, 9H).

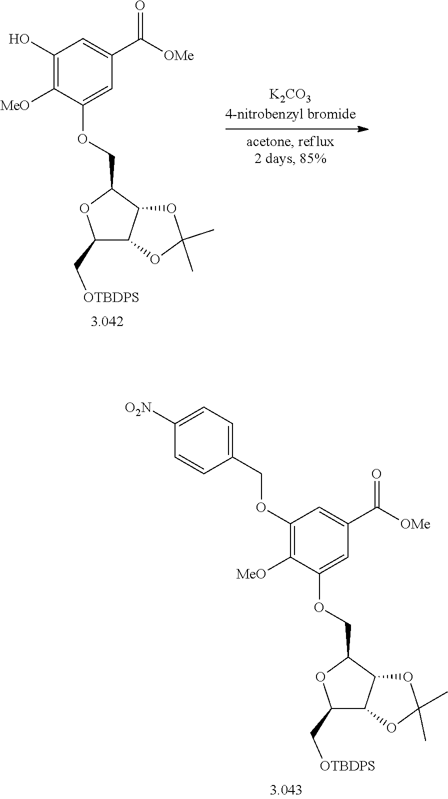

To a solution of 3.042 (140 mg, 0.23 mmol) in acetone (10 mL) was added 4-nitrobenzyl bromide (100 mg, 0.46 mmol) and K₂CO₃ (310 mg, 2.24 mmol) at 25° C. The reaction mixture was then heated to reflux for 2 days. Upon cooling to 25° C., the reaction was quenched with an addition of saturated aqueous NH₄Cl, and the resulting mixture was diluted with EtOAc. The layers were separated, and the aqueous layer was extracted with EtOAc. The combined organic layers were dried over anhydrous Na2SO4, filtered, and concentrated in vacuo. The residue was purified by column chromatography (silica gel, hexanes/EtOAc, 7/2) to afford 3.043 (148 mg, 85%). ¹H NMR (400 MHz, CDCl₃) δ 8.25 (d, J=8.6 Hz, 2H), 7.66-7.61 (m, 6H), 7.38-7.29 (m, 8H), 5.24 (s, 2H), 4.74 (dd, J=6.4, 3.4 Hz, 1H), 4.68 (dd, J=6.2, 4.2 Hz, 1H), 4.39 (ddd, J=4.6, 4.6, 4.4 Hz, 1H), 4.20 (ddd, J=4.2, 4.0, 4.0 Hz, 1H), 4.16-4.09 (m, 2H), 3.86 (s, 3H), 3.85 (s, 3H), 3.80 (t, J=4.4 Hz, 2H), 1.57 (s, 6H), 1.26 (s, 3H), 1.05 (s, 9H).

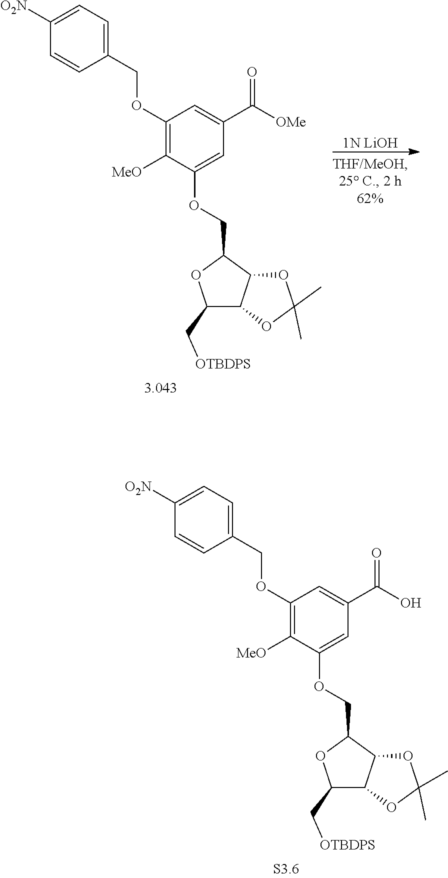

To a solution of 3.043 (60 mg, 0.08 mmol), in THF/MeOH (3/1) (3 mL) was added 1 N LiOH (0.6 mL) at 25° C. After stirring for 2 h, 1 M HCl was added until the solution became acidic, and the resulting mixture was diluted with EtOAc. The layers were separated, and the aqueous layer was extracted with EtOAc. The combined organic layers were dried over anhydrous Na₂SO₄, filtered, and concentrated in vacuo. The residue was purified by column chromatography (silica gel, hexane/EtOAc, 1/4) to afford S3.6 (36 mg, 62%). ¹H NMR (500 MHz, CDCl₃) δ 8.26 (d, J=8.6 Hz, 2H), 7.67-7.62 (m, 6H), 7.39-7.31 (m, 8H), 5.25 (s, 2H), 4.74 (dd, J=6.4, 3.4 Hz, 1H), 4.68 (dd, J=6.4, 4.2 Hz, 1H), 4.39 (ddd, J=4.7, 4.7, 4.6 Hz, 1H), 4.22-4.19 (m, 1H), 4.18-4.10 (m, 2H), 3.88 (s, 3H), 3.81 (t, J=4.7 Hz, 2H), 1.57 (s, 3H), 1.37 (s, 3H), 1.05 (s, 9H).

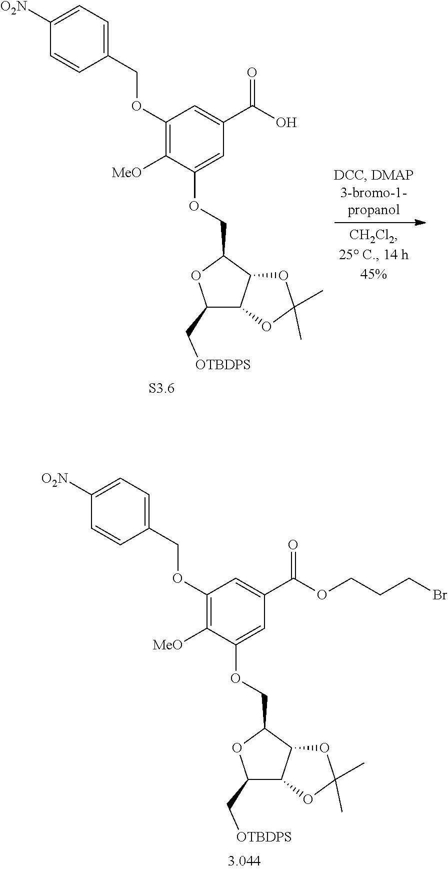

To a solution of S3.6 (30 mg, 0.04 mmol) in CH₂Cl₂ (1 mL) was added 3-bromo-1-propanol (4 μL, 0.044 mmol), DCC (10 mg, 0.048 mmol), and DMAP (0.6 mg) at 25° C. After stirring for 14 h, the reaction was concentrated in vacuo. The residue was purified by column chromatography (silica gel, hexanes/EtOAc, 6/1) to afford 3.044 (15 mg, 45%). ¹H NMR (500 MHz, CDCl₃) δ 8.26 (d, J=8.6 Hz, 2H), 7.67-7.62 (m, 6H), 7.40-7.28 (m, 8H), 5.25 (s, 2H), 4.74 (dd, J=6.5, 3.5 Hz, 1H), 4.68 (dd, J=6.5, 4.2 Hz, 1H), 4.41 (t, J=6.1 Hz, 2H), 4.39-4.37 (m, 1H), 4.20 (ddd, J=4.2, 4.1, 4.1 Hz, 1H), 4.18-4.12 (m, 2H), 3.87 (s, 3H), 3.83-3.79 (m, 2H), 3.46 (t, J=6.5 Hz, 2H), 2.27 (dddd, J=6.3, 6.3, 6.3, 6.3 Hz, 2H), 1.57 (s, 3H), 1.25 (s, 3H), 1.05 (s, 9H).

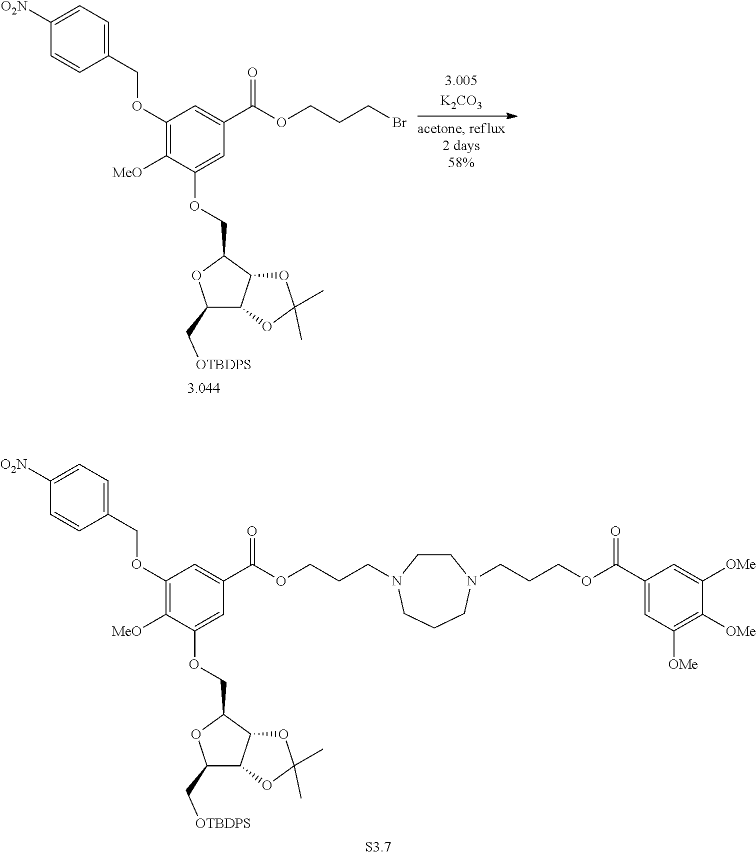

To a solution of 3.044 (9 mg, 0.01 mmol) in acetone (0.5 mL) was added 3.005 (10 mg, 0.03 mmol) and K₂CO₃ (20 mg, 0.1 mmol) at 25° C. The reaction mixture was then warmed to reflux for 2 days. Upon cooling to 25° C., the reaction was quenched with the addition of saturated aqueous NH₄Cl, and the resulting mixture was diluted with EtOAc. The layers were separated, and the aqueous layer was extracted with EtOAc. The combined organic layers dried over anhydrous Na₂SO₄, filtered, and concentrated in vacuo. The residue was purified by column chromatography (silica gel, CH₂Cl₂/MeOH, 30/1 to 20/1) to afford S3.7 (7 mg, 58%). ¹H NMR (500 MHz, CDCl₃) δ 8.25 (d, J=8.7 Hz, 2H), 7.66-7.63 (m, 6H), 7.39-7.28 (m, 10H), 5.25 (s, 2H), 4.73 (dd, J=6.4, 3.5 Hz, 1H), 4.68 (dd, J=6.3, 4.3 Hz, 1H), 4.38-4.26 (m, 3H), 4.32 (t, J=6.5 Hz, 2H), 4.20-4.12 (m, 3H), 3.90 (s, 9H), 3.85 (s, 3H), 3.79 (t, J=4.1 Hz, 2H), 2.96-2.59 (m, 12H), 2.08-1.85 (m, 6H), 1.56 (s, 3H), 1.37 (s, 3H), 1.04 (s, 9H).

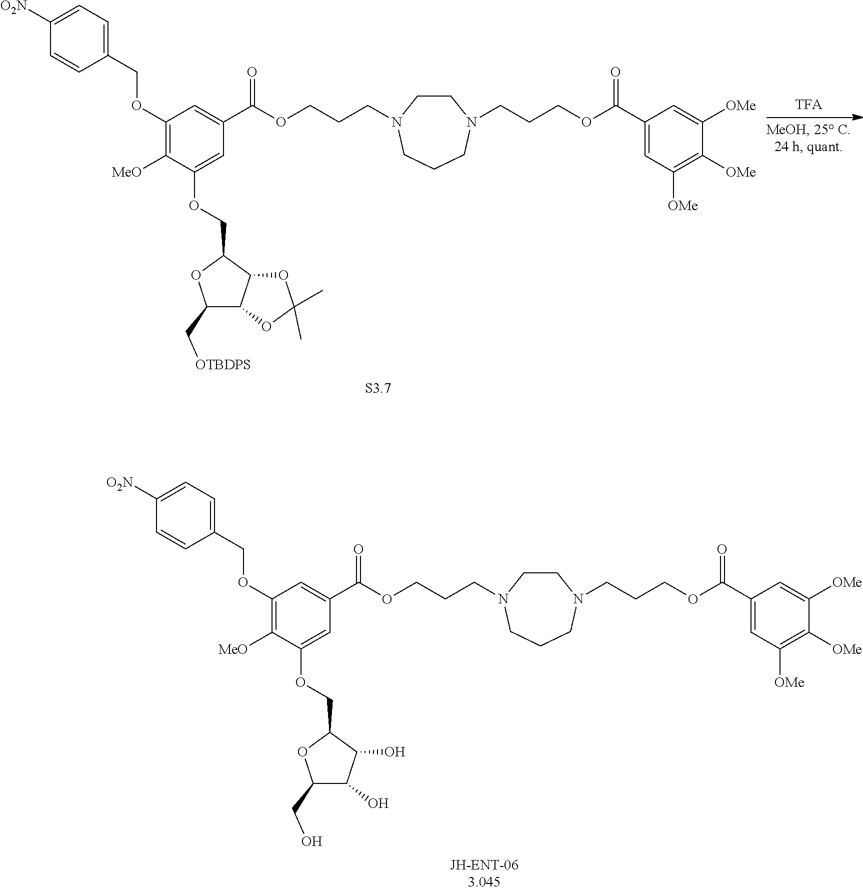

To a solution of S3.7 (7 mg, 0.006 mmol) in MeOH (1 mL) was added TFA (0.2 mL) at 25° C. After stirring for 24 h, the reaction was concentrated in vacuo to provide 3.045 (5 mg, quantitative). ¹H NMR (500 MHz, CDCl₃) δ 8.26 (d, J=8.7 Hz, 2H), 8.65 (d, J=8.7 Hz, 2H), 7.35 (br s, 1H), 7.31 (br s, 1H), 7.26 (s, 2H), 5.25 (s, 2H), 4.41 (t, J=5.9 Hz, 2H), 4.38-4.29 (m, 5H), 4.22-4.20 (m, 1H), 4.18-4.16 (m, 1H), 4.03 (br s, 1H), 3.94 (s, 3H), 3.91 (s, 3H), 3.91 (s, 6H), 3.85-3.82 (m, 4H), 3.63 (dd, J=12.4, 3.2 Hz, 1H), 3.60-3.52 (m, 3H), 3.33-3.29 (m, 2H), 3.28-3.24 (m, 2H), 2.51-2.49 (m, 2H), 2.29-2.22 (m, 6H).

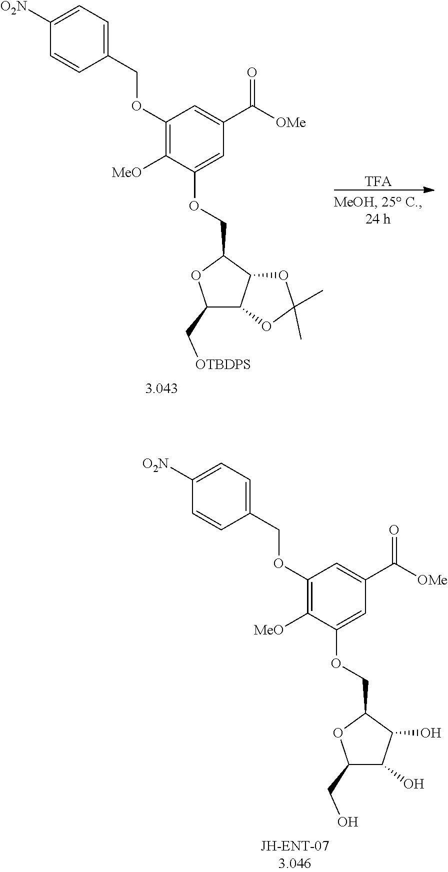

To a solution of 3.043 (50 mg, 0.066 mmol) in MeCOH (4 mL) was added TFA (1 mL) at 25° C. After stirring for 24 h, the reaction was concentrated in vacuo to provide 3.046 (31 mg, quantitative). ¹H NMR (500 MHz, CDCl₃) δ 8.26 (d, J=8.6 Hz, 2H), 7.63 (d, J=8.4 Hz, 2H), 7.36 (d, J=1.8 Hz, 1H), 7.34 (d, J=1.8 Hz, 1H), 5.25 (s, 2H), 4.48 (t, J=6.1 Hz, 1H), 4.32 (dd, J=5.5, 3.2 Hz, 1H), 4.28 (d, J=2.9 Hz, 2H), 4.19 (ddd, J=6.2, 2.9, 2.9, 1H), 4.09 (ddd, J=2.9, 2.9, 2.8, 1H), 3.92 (s, 3H), 3.90 (s, 3H), 3.89-3.86 (m, 1H), 3.69 (dd, J=12.4, 2.8 Hz, 1H).

1. A compound of formula (I), or a pharmaceutically acceptable salt thereof: 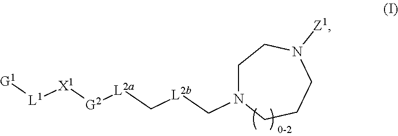
wherein:
G¹ is a 6- to 12-membered aryl or a 5- to 12-membered heteroaryl, wherein G¹ is optionally substituted with 1-4 R^{1x}, wherein, at each occurrence, R^{1x} is independently —NO₂, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, or —C₁₋₃alkylene-Q^{1a};
R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{1a} at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L¹ is C₁₋₄alkylene or absent;
X¹ is O, S, or NH;
G² is phenylene, wherein G² is optionally substituted with 1-4 R^{2x}, wherein, at each occurrence, R^{2x} is independently halogen, C₁₋₆alkyl, C₁₋₆haloalkyl, C₂₋₆alkenyl, —OR^{2a}, —NO₂, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, or —C₁₋₃alkylene-Q^{2a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a};
R^{2d}, at each occurrence, is independently C₁₋₆alkyl, C₁₋₆haloalkyl, G^{2a}, or —C₁₋₃alkylene-G^{2a};
G^{2a}, at each occurrence, is independently a C₃₋₆cycloalkyl, a phenyl, a 4- to 6-membered heterocyclyl, or a 5- to 6-membered heteroaryl; wherein G^{2a} is optionally substituted with 1-5 substituents independently selected from the group consisting of halogen, oxo, C₁₋₄alkyl, —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{2a}, at each occurrence, is independently —OC₁₋₄alkyl, —OC₁₋₄haloalkyl, —OH, —NH₂, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L^{2a} is 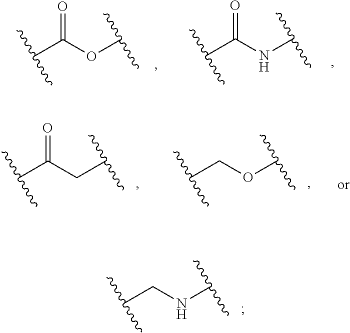
L^{2b} is C₁₋₃alkylene, C₂₋₃alkenylene, C₂₋₃alkynylene, or absent;
Z¹ is 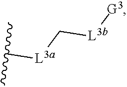
C₁₋₆alkyl, or C₃₋₆cycloalkyl;
L^{3a} is C₁₋₃alkylene, C₂₋₃alkenylene, C₂₋₃alkynylene, or absent; and
L^{3b} is 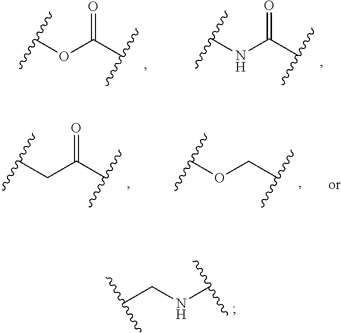
G³ is a 6- to 12-membered aryl or a 5- to 12-membered heteroaryl, wherein G³ is optionally substituted with 1-4 R^{3x}, wherein, at each occurrence, R^{3x} is independently halogen, C₁₋₆alkyl, C₁₋₆haloalkyl, C₂₋₆alkenyl, —OR^{3a}, —NO₂, —NR^{3a}R^{3b}, —SR^{3a}, —NR^{3a}C(O)R^{3c}, cyano, —C(O)OR^{3a}, —C(O)NR^{3a}R^{3b}, —C(O)R^{3c}, —SO₂R^{3d}, —SO₂NR^{3a}R^{3b}, G^{3a}, —C₁₋₃alkylene-G^{3a}, or —C₁₋₃alkylene-Q^{3a};
R^{3a}, R^{3b}, and R^{3c}, at each occurrence, are each independently hydrogen, C₁₋₆alkyl, C₁₋₆haloalkyl, G^{3a}, or —C₁₋₃alkylene-G^{3a};
R^{3d}, at each occurrence, is independently C₁₋₆alkyl, C₁₋₆haloalkyl, G^{3a}, or —C₁₋₃alkylene-G^{3a};
G^{3a}, at each occurrence, is independently a C₃₋₈cycloalkyl, a 4- to 12-membered heterocyclyl, a 6- to 12-membered aryl, or a 5- to 12-membered heteroaryl, wherein G^{3a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂; and
Q^{3a} at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄ alkyl, or —C(O)N(C₁₋₄alkyl)₂.

2. (canceled)

3. The compound of claim 1, or a pharmaceutically acceptable salt thereof, wherein G¹ and G³ are each phenyl.

4-5. (canceled)

6. The compound of claim 1, or a pharmaceutically acceptable salt thereof, wherein L¹ is C₁₋₃alkylene, L^{2a} is 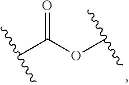 [SMILES: *OC(*)=O]
L^{2b} is C₁₋₃alkylene, L^{3a} is C₁₋₃alkylene, and L^{3b} is

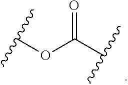 [SMILES: *OC(*)=O]

7. The compound of claim 1, or a pharmaceutically acceptable salt thereof, wherein X¹ is O or S and

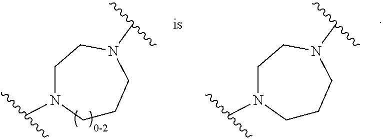

8-21. (canceled)

22. The compound of claim 1, or a pharmaceutically acceptable salt thereof, wherein the compound is:

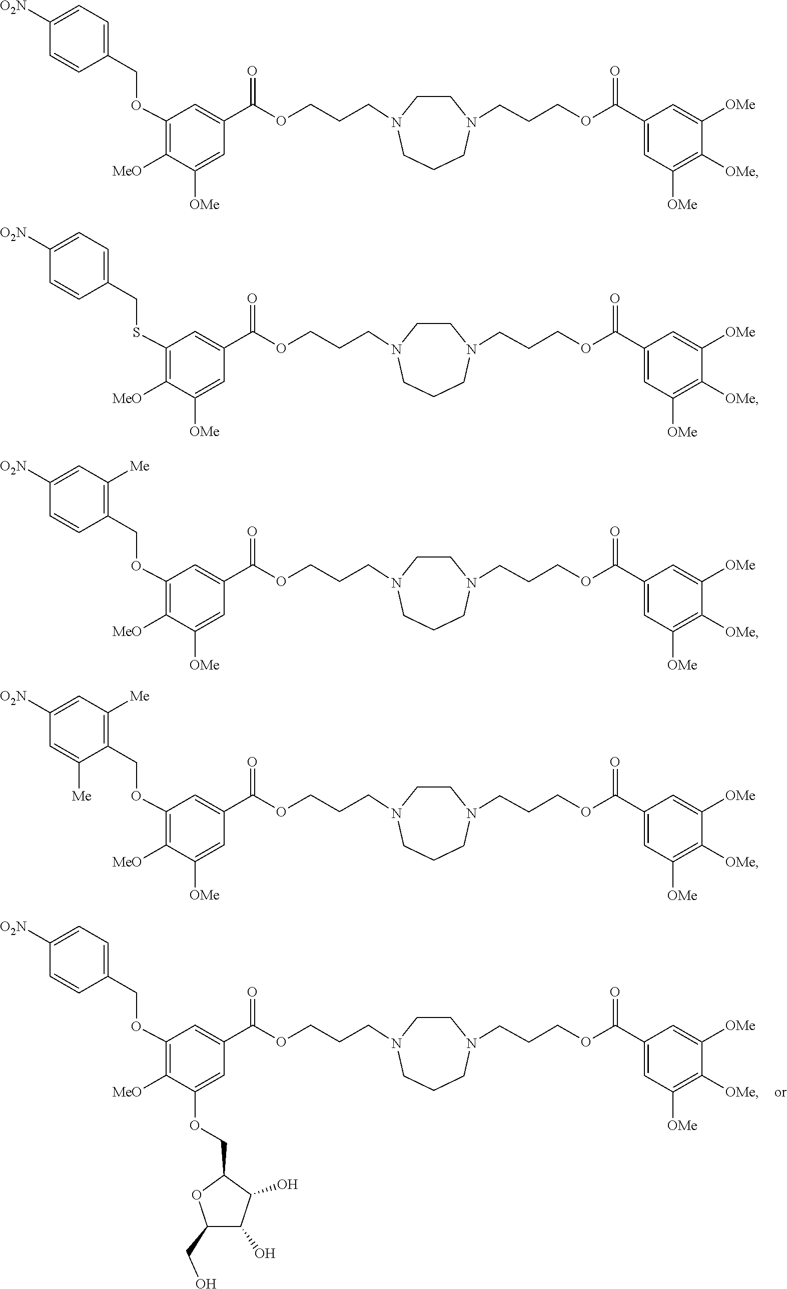 [SMILES: COc1cc(C(=O)OCCCN2CCCN(CCCOC(=O)c3cc(OC)c(OC)c(OCc4ccc([N+](=O)[O-])cc4)c3)CC2)cc(OC)c1OC ; COc1cc(C(=O)OCCCN2CCCN(CCCOC(=O)c3cc(OC)c(OC)c(SCc4ccc([N+](=O)[O-])cc4)c3)CC2)cc(OC)c1OC ; COc1cc(C(=O)OCCCN2CCCN(CCCOC(=O)c3cc(OC)c(OC)c(OCc4ccc([N+](=O)[O-])cc4C)c3)CC2)cc(OC)c1OC ; COc1cc(C(=O)OCCCN2CCCN(CCCOC(=O)c3cc(OC)c(OC)c(OCc4c(C)cc([N+](=O)[O-])cc4C)c3)CC2)cc(OC)c1OC ; COc1cc(C(=O)OCCCN2CCCN(CCCOC(=O)c3cc(OCc4ccc([N+](=O)[O-])cc4)c(OC)c(OC[C@@H]4O[C@H](CO)[C@@H](O)[C@H]4O)c3)CC2)cc(OC)c1OC]

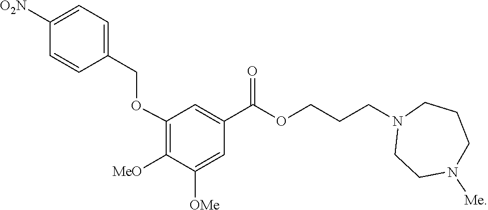 [SMILES: COc1cc(C(=O)OCCCN2CCCN(C)CC2)cc(OCc2ccc([N+](=O)[O-])cc2)c1OC]

23. A pharmaceutical composition comprising the compound of claim 1, or a pharmaceutically acceptable salt thereof, and a pharmaceutically acceptable carrier.

24. A method of treating neuropathic pain, the method comprising administering to a subject in need thereof a therapeutically effective amount of the compound of claim 1, or a pharmaceutically acceptable salt thereof, or the pharmaceutical composition of claim 23.

25. A method of inhibiting an equilibrative nucleoside transporter (ENT), the method comprising administering to a subject in need thereof a therapeutically effective amount of the compound of claim 1, or a pharmaceutically acceptable salt thereof, or the pharmaceutical composition of claim 23.

26. (canceled)

27. A compound of formula (II), or a pharmaceutically acceptable salt thereof: 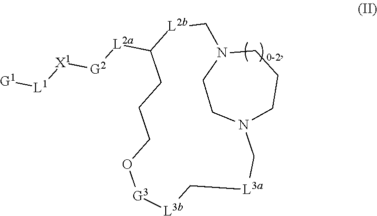
wherein:
G¹ is a 6- to 12-membered aryl or a 5- to 12-membered heteroaryl, wherein G¹ is optionally substituted with 1-4 R^{1x}, wherein, at each occurrence, R^{1x} is independently —NO₂, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, or —C₁₋₃alkylene-Q^{1a};
R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{1a} at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L¹ is C₁₋₄alkylene or absent;
X¹ is O, S, or NH;
when G¹-L¹-X¹ is present G² is phenylene;
when G¹-L¹-X¹ is absent G² is phenyl;
G² is optionally substituted with 1-4 R^{2x}, wherein, at each occurrence, R^{2x} is independently halogen, C₁₋₆alkyl, C₁₋₆haloalkyl, C₂₋₆alkenyl, —OR^{2a}, —NO₂, —NR^{2a}R^{2b}, —SR^{2a}, —NR^{2a}C(O)R^{2c}, cyano, —C(O)OR^{2a}, —C(O)R^{2c}, —C(O)NR^{2a}R^{2b}, —SO₂NR^{2a}R^{2b}, —SO₂R^{2d}, G^{2a}, —C₁₋₃alkylene-G^{2a}, or —C₁₋₃alkylene-Q^{2a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a};
R^{2d}, at each occurrence, is independently C₁₋₆alkyl, C₁₋₆haloalkyl, G^{2a}, or —C₁₋₃alkylene-G^{2a};
G^{2a}, at each occurrence, is independently a C₃₋₆cycloalkyl, a phenyl, a 4- to 6-membered heterocyclyl, or a 5- to 6-membered heteroaryl; wherein G^{2a} is optionally substituted with 1-5 substituents independently selected from the group consisting of halogen, oxo, C₁₋₄alkyl, —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{2a}, at each occurrence, is independently —OC₁₋₄alkyl, —OC₁₋₄haloalkyl, —OH, —NH₂, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L^{2a} is 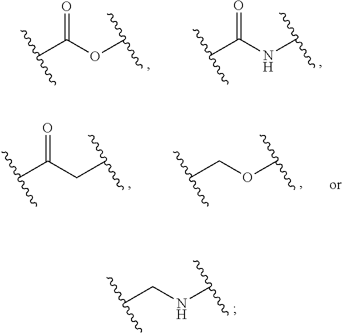
L^{2b} is C₁₋₃alkylene, C₂₋₃alkenylene, C₂₋₃alkynylene, or absent;
L^{3a} is C₁₋₃alkylene, C₂₋₃alkenylene, C₂₋₃alkynylene, or absent; and
L^{3b} is 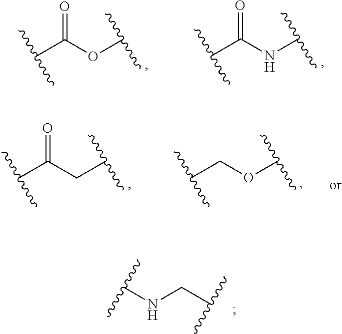
G³ is a 6- to 12-membered arylene or a 5- to 12-membered heteroarylene, wherein G³ is optionally substituted with 1-4 R^{3x}, wherein, at each occurrence, R^{3x} is independently halogen, C₁₋₆alkyl, C₁₋₆haloalkyl, C₂₋₆alkenyl, —OR^{3a}, —NO₂, —NR^{3a}R^{3b}, —SR^{3a}, —NR^{3a}C(O)R^{3c}, cyano, —C(O)OR^{3a}, —C(O)NR^{3a}R^{3b}, —C(O)R^{3c}, —SO₂R^{3d}, —SO₂NR^{3a}R^{3b}, G^{3a}, —C₁₋₃alkylene-G^{3a}, or —C₁₋₃alkylene-Q^{3a};
R^{3a}, R^{3b}, and R^{3c}, at each occurrence, are each independently hydrogen, C₁₋₆alkyl, C₁₋₆haloalkyl, G^{3a}, or —C₁₋₃alkylene-G^{3a};
R^{3d}, at each occurrence, is independently C₁₋₆alkyl, C₁₋₆haloalkyl, G^{3a}, or —C₁₋₃alkylene-G^{3a};
G^{3a}, at each occurrence, is independently a C₃₋₈cycloalkyl, a 4- to 12-membered heterocyclyl, a 6- to 12-membered aryl, or a 5- to 12-membered heteroaryl, wherein G^{3a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂; and
Q^{3a}, at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂.

28. (canceled)

29. The compound of claim 27, or a pharmaceutically acceptable salt thereof, wherein G¹ is phenyl and G³ is phenylene.

30. (canceled)

31. The compound of claim 27, or a pharmaceutically acceptable salt thereof, wherein 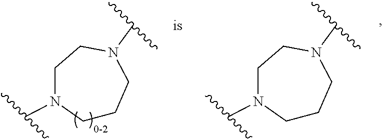
L¹ is C₁₋₄alkylene, L^{2a} is 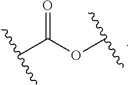 [SMILES: *OC(*)=O]
L^{2b} is C₁₋₃alkylene, L^{3a} is C₁₋₃alkylene, L^{3b} is 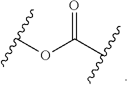 [SMILES: *OC(*)=O]
and X¹ is O or S.

32-38. (canceled)

39. The compound of claim 27, or a pharmaceutically acceptable salt thereof, wherein the compound is:

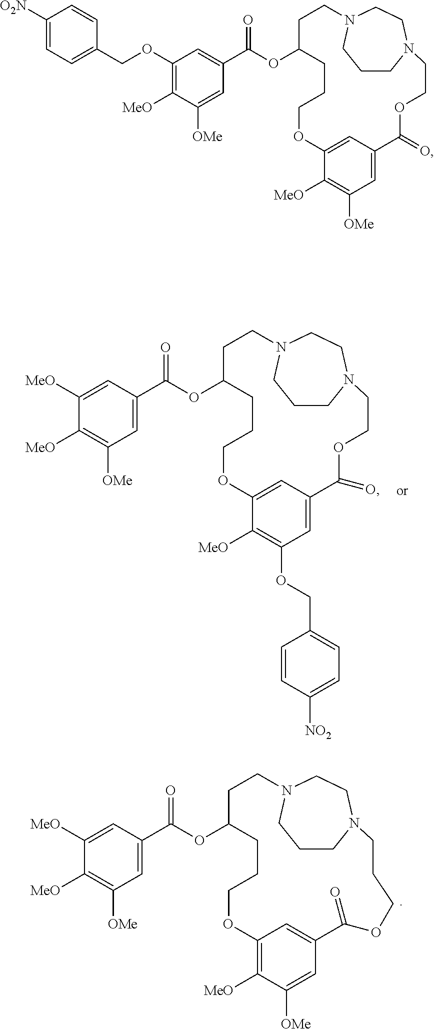

40. A pharmaceutical composition comprising the compound of claim 27, or a pharmaceutically acceptable salt thereof, and a pharmaceutically acceptable carrier.

41. A method of treating neuropathic pain, the method comprising administering to a subject in need thereof a therapeutically effective amount of the compound of claim 27, or a pharmaceutically acceptable salt thereof, or the pharmaceutical composition of claim 40.

42. A method of inhibiting an equilibrative nucleoside transporter (ENT), the method comprising administering to a subject in need thereof a therapeutically effective amount of the compound of claim 27, or a pharmaceutically acceptable salt thereof, or the pharmaceutical composition of claim 40.

43. A compound of formula (III), or a pharmaceutically acceptable salt thereof: 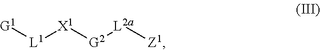
wherein:
G¹ is a 6- to 12-membered aryl or a 5- to 12-membered heteroaryl, wherein G¹ is optionally substituted with 1-4 R^{1x}, wherein, at each occurrence, R^{1x} is independently —NO₂, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, or —C₁₋₃alkylene-Q^{1a};
R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, —OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{1a} at each occurrence, is independently —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L¹ is C₁₋₄alkylene or absent;
X¹ is O, S, or NH;
G² is phenylene, wherein G² is optionally substituted with 1-4 R^{2x}, wherein, at each occurrence, R^{2x} is independently halogen, C₁₋₆alkyl, C₁₋₆haloalkyl, C₂₋₆alkenyl, —OR^{2a}, —NO₂, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, or —C₁₋₃alkylene-Q^{2a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a};
R^{2d}, at each occurrence, is independently C₁₋₆alkyl, C₁₋₆haloalkyl, G^{2a}, or —C₁₋₃alkylene-G^{2a};
G^{2a}, at each occurrence, is independently a C₃₋₆cycloalkyl, a phenyl, a 4- to 6-membered heterocyclyl, or a 5- to 6-membered heteroaryl; wherein G^{2a} is optionally substituted with 1-5 substituents independently selected from the group consisting of halogen, oxo, C₁₋₄alkyl, —OC₁₋₄alkyl, —OC₁₋₄haloalkyl, —OH, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q^{2a}, at each occurrence, is independently —OC₁₋₄alkyl, —OC₁₋₄haloalkyl, —OH, —NH₂, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
L^{2a} is 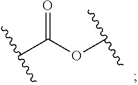 [SMILES: *OC(*)=O]
and
Z¹ is C₁₋₆alkyl, or C₃₋₆cycloalkyl.

44-45. (canceled)

46. The compound of claim 43, or a pharmaceutically acceptable salt thereof, wherein the compound is:

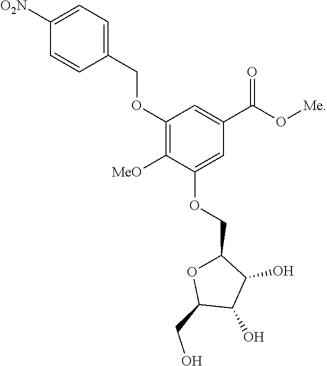 [SMILES: COC(=O)c1cc(OCc2ccc([N+](=O)[O-])cc2)c(OC)c(OC[C@@H]2O[C@H](CO)[C@@H](O)[C@H]2O)c1]

47. A pharmaceutical composition comprising the compound of claim 43, or a pharmaceutically acceptable salt thereof, and a pharmaceutically acceptable carrier.

48. A method of treating neuropathic pain, the method comprising administering to a subject in need thereof a therapeutically effective amount of the compound of claim 43, or a pharmaceutically acceptable salt thereof, or the pharmaceutical composition of claim 47.

49. A method of inhibiting an equilibrative nucleoside transporter (ENT), the method comprising administering to a subject in need thereof a therapeutically effective amount of the compound of claim 43, or a pharmaceutically acceptable salt thereof, or the pharmaceutical composition of claim 47.

50. (canceled)
